(19) United States
(12) Patent Application Publication
(10) Pub. No.: US 2022/0160802 A1
(43) Pub. Date: May 26, 2022

(54) METHODS AND COMPOSITIONS FOR PROMOTING HEALTH IN A SUBJECT

(71) Applicant: COMMONWEALTH SCIENTIFIC AND INDUSTRIAL RESEARCH ORGANISATION, Acton (AU)

(72) Inventors: Mary Ann AUGUSTIN, Acton (AU); Netsanet SHIFERAW TEREFE, Acton (AU); Michael CONLON, Acton (AU); Paul BLATCHFORD, Acton (AU)

(73) Assignee: COMMONWEALTH SCIENTIFIC AND INDUSTRIAL RESEARCH ORGANISATION, Acton (AU)

(21) Appl. No.: 17/594,130

(22) PCT Filed: Apr. 3, 2020
(86) PCT No.: PCT/AU2020/050338

§ 371 Date: Oct. 4, 2021

(30) Foreign Application Priority Data
Apr. 3, 2019 (AU) 2019901142

(51) Int. Cl. A61K 36/31; A61K 31/26; A61K 35/747; A61K 35/60; A61K 45/06; A61K 9/00; A61K 9/107

(52) U.S. Cl. CPC A61K 36/31; A61K 31/26; A61K 35/747; A61K 35/60; A61K 2035/115; A61K 9/0031; A61K 9/0014; A61K 9/0053; A61K 9/107; A61K 45/06

(57) ABSTRACT

The present invention provides methods for promoting health in a subject, comprising administering to a subject a Brassicaceae product fermented with lactic acid bacteria. In addition the present invention compositions and delivery vehicles for promoting health in a subject, comprising administering to a subject a Brassicaceae product fermented with lactic acid bacteria.

### FIELD OF THE INVENTION

The present invention provides methods, compositions and delivery vehicles for promoting health in a subject, comprising administering to a subject a fermented Brassicaceae product.

### BACKGROUND OF THE INVENTION

The prevalence of chronic diseases such as obesity, cardiovascular disease, metabolic syndrome, inflammatory diseases, autoimmune diseases, diabetes, gut health conditions and certain cancers is increasing globally, fuelled particularly by dramatic rises in developing countries where growing affluence is also associated with an expanding adoption of more Westernised diet and lifestyle patterns. While over consumption of high calorie, easily digested foods and beverages plays an important role, there is growing evidence that changes to the 10¹⁴ microbes, comprising over 10³ bacterial species (collectively the gut microbiota) of the human large bowel, driven by these dietary patterns also makes a contribution and provides target for preventive and clinical intervention. Diet plays a big part in feeding this hungry gut microbiota, shaping both its structure (the relative proportions of the different species) and its function (genes expressed, metabolites made and their interaction with the subject). Dietary fibre is fermented by the gut microbiota into short chain fatty acids (SCFA). SCFA positively influence the gastrointestinal microenvironment (increases gut health) and other organ sites in the body as they are small enough to enter the blood stream and can be distributed to other sites in the body.

Accordingly, there is a requirement for supplements and nutritional agents that promote health in a subject.

### SUMMARY OF THE INVENTION

The present inventors have developed methods, compositions and delivery vehicles for promoting health in a subject.

In an aspect, the invention provides a method of promoting health in a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

In an embodiment, the Brassicaceae product increases the gastrointestinal level of one or more short chain fatty acids (SCFA) in the subject.

In an embodiment, Brassicaceae product increases the production of one or more SCFA in the gastrointestinal tract in the subject. In an embodiment, the Brassicaceae product increases the production of one or more SCFA in the lower gastrointestinal tract of the subject. In an embodiment, the Brassicaceae product increases the production of one or more SCFA of the colon of the subject. In an embodiment, production of one or more SCFA is increased relative to an unfermented Brassicaceae product.

In an embodiment, the Brassicaceae product comprises an isothiocyanate.

In an embodiment, the Brassicaceae product comprises live lactic acid bacteria from Brassicaceae.

In an aspect, the invention provides a method of promoting the health of the gut microbiome in a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

In an aspect, the invention provides a method of treating and/or preventing microbial dysbiosis in the gastrointestinal tract of a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

In an aspect, the invention provides a method of treating and/or preventing inflammation in a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

In an aspect, the invention provides a method of treating and/or preventing diabetes in a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

In an aspect, the invention provides use of a Brassicaceae product fermented with lactic acid bacteria in the manufacture of a medicament for promoting health in a subject, wherein the lactic acid bacteria were derived from an isolate obtained from Brassicaceae and/or the Brassicaceae product was pre-treated prior to fermentation.

In an aspect, the invention provides use of a Brassicaceae product fermented with lactic acid bacteria in the manufacture of a medicament for promoting health of the gut microbiome in a subject, wherein the lactic acid bacteria were derived from an isolate obtained from Brassicaceae and/or the Brassicaceae product was pre-treated prior to fermentation.

In an aspect, the invention provides use of a Brassicaceae product fermented with lactic acid bacteria in the manufacture of a medicament for treating and/or preventing microbial dysbiosis in the gastrointestinal tract of a subject, wherein the lactic acid bacteria were derived from an isolate obtained from Brassicaceae and/or the Brassicaceae product was pre-treated prior to fermentation.

In an aspect, the invention provides use of a Brassicaceae product fermented with lactic acid bacteria in the manufacture of a medicament for treating and/or preventing inflammation in a subject, wherein the lactic acid bacteria were derived from an isolate obtained from Brassicaceae and/or the Brassicaceae product was pre-treated prior to fermentation.

In an aspect, the invention provides use of a Brassicaceae product fermented with lactic acid bacteria in the manufacture of a medicament for treating and/or preventing diabetes in a subject, wherein the lactic acid bacteria were derived from an isolate obtained from Brassicaceae and/or the Brassicaceae product was pre-treated prior to fermentation.

In an aspect, the invention provides a pharmaceutical composition comprising a Brassicaceae product fermented with lactic acid bacteria, wherein the lactic acid bacteria were derived from an isolate obtained from Brassicaceae and/or the Brassicaceae product was pre-treated prior to fermentation for use in promoting health in a subject.

In an aspect, the invention provides a pharmaceutical composition comprising a Brassicaceae product fermented with lactic acid bacteria, wherein the lactic acid bacteria were derived from an isolate obtained from Brassicaceae and/or the Brassicaceae product was pre-treated prior to fermentation for use in promoting health of the gut microbiome in a subject.

In an aspect, the invention provides a pharmaceutical composition comprising a Brassicaceae product with lactic acid bacteria, wherein the lactic acid bacteria were derived from an isolate obtained from Brassicaceae and/or the Brassicaceae product was pre-treated prior to fermentation for treating and/or preventing microbial dysbiosis in the gastrointestinal tract of a subject.

In an aspect, the invention provides a pharmaceutical composition comprising a Brassicaceae product fermented with lactic acid bacteria, wherein the lactic acid bacteria were derived from an isolate obtained from Brassicaceae and/or the Brassicaceae product was pre-treated prior to fermentation for treating and/or preventing inflammation in a subject.

In an aspect, the invention provides a pharmaceutical composition comprising a Brassicaceae product fermented with lactic acid bacteria, wherein the lactic acid bacteria were derived from an isolate obtained from Brassicaceae and/or the Brassicaceae product was pre-treated prior to fermentation for treating and/or preventing diabetes in a subject.

In an aspect, the invention provides a prebiotic composition comprising a Brassicaceae product fermented with lactic acid bacteria, wherein the lactic acid bacteria were derived from an isolate obtained from Brassicaceae and/or the Brassicaceae product was pre-treated prior to fermentation, wherein the prebiotic increases the gastrointestinal level of one or more SCFA in a subject.

In an aspect, the invention provides a synbiotic composition comprising a Brassicaceae product fermented with lactic acid bacteria, wherein the lactic acid bacteria were derived from an isolate obtained from Brassicaceae and/or the Brassicaceae product was pre-treated prior to fermentation, wherein the prebiotic increases the gastrointestinal level of one or more SCFA in a subject.

In an aspect, the invention provides a combined prebiotic and probiotic composition comprising:

i) a Brassicaceae product fermented with lactic acid bacteria, wherein the lactic acid bacteria were derived from an isolate obtained from Brassicaceae and/or the Brassicaceae product was pre-treated prior to fermentation; and

ii) live lactic acid bacteria.

In an aspect, the invention provides a faecal microbiota suitable for transplantation into a subject, wherein the faecal microbiota is isolated from a subject administered a Brassicaceae product fermented with lactic acid bacteria, wherein the lactic acid bacteria were derived from an isolate obtained from Brassicaceae and/or the Brassicaceae product was pre-treated prior to fermentation.

In an aspect, the invention provides a delivery vehicle for delivering a bioactive to a subject, wherein the delivery vehicle comprises a Brassicaceae product fermented with lactic acid bacteria, wherein the lactic acid bacteria were derived from an isolate obtained from Brassicaceae and/or the Brassicaceae product was pre-treated prior to fermentation.

In an embodiment, the bioactive is selected from one or more or all of: i) a fatty acid, ii) oil, iii) a further prebiotic, and iv) a further probiotic.

In an aspect, the present invention provides a method of preparing a Brassicaceae product comprising:

i) fermenting Brassicaceae material with lactic acid bacteria;

ii) adding a fatty acid and/or oil before or during step i).

In an embodiment, the method further comprises forming an emulsion or suspension.

In an embodiment, the Brassicaceae material is pre-treated.

In an embodiment, pre-treating comprises one or more of: i) heating; ii) macerating; iii) microwaving; iv) exposure to high frequency sound waves (ultrasound), v) pulse electric field processing; and vi) high pressure processing.

In an embodiment, pre-treating comprising heating and maceration. In an embodiment, heating occurs before macerating or wherein heating and macerating occur at the same time. In an embodiment, pre-treating comprises heating the Brassicaceae material to a temperature of about 50° C. to about 70° C. followed by maceration.

In an embodiment, the lactic acid bacteria were derived from an isolate obtained from Brassicaceae.

In an aspect, the present invention provides an emulsion or suspension produced by the methods as described herein.

In an aspect, the present invention provides a Brassicaceae product comprising the emulsion or suspension as described herein.

Any embodiment herein shall be taken to apply mutatis mutandis to any other embodiment unless specifically stated otherwise. For instance, as the skilled person would understand examples of lactic acid bacteria outlined above for the methods of the invention equally apply to products of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. A) Shows the pathways of hydrolysis of glucoraphanin to sulforaphane and sulforaphane nitrile. B) Shows the effects of maceration and fermentation on sulforaphane content (mg/kg, DW) in broccoli puree. C) Shows the effect of fermentation on lactic acid bacteria count (log CFU/gm) of broccoli puree during storage.

FIG. 2. A) Shows the effects of fermentation on the stability of sulforaphane in broccoli puree stored at 4° C. and 25° C. (RT). B) Shows the effects of heat treatment condition on the conversion of glucoraphanin into sulforaphane in broccoli matrix.

FIG. 3. A) Shows the total phenolic content (mg GAE/100 g DW) of raw broccoli and its changes during fermentation and storage at 25° C. and 4° C., respectively. B) Shows the ORAC (oxygen radical absorbance capacity) antioxidant capacity (μmol TE/g DW) of raw broccoli and its changes during fermentation and storage at 25° C. and 4° C., respectively.

FIG. 4. Shows the fermentation time taken to reach a pH of 4.4 or lower for different combinations of lactic acid bacteria strains.

FIG. 5. A) Shows sulforaphane yield (μmol/kg DW) under different heat treatment conditions of broccoli with a sealed bag. B) Shows sulforaphane yield (μmol/kg DW) under different heat treatment conditions of broccoli immersed directly in water.

FIG. 6. Shows the comparative effects of the combined effects of maceration, pre-heating and fermentation with just maceration and preheating and maceration, preheating and chemical acidification on sulforaphane yield (μmol/kg DW) just after processing and during storage at 4° C. and 25° C. Samples were pre-treated at 65° C. for 3 min in sealed packs.

FIG. 7. Shows the effect of fermentation and storage on glucoraphanin content. Glucoraphanin content is reduced in fermented samples stored at 25° C. and 4° C. compared to raw samples.

FIG. 8. PLS-DA score plot showing the difference in polyphenolic metabolite profile of raw and fermented broccoli puree.

FIG. 9. Important features differentiating fermented and non-fermented samples identified by PLS-DA. The boxes on the right indicate the relative concentration of the respective metabolites in each group.

FIG. 10. Shows the effect of lactic acid fermentation on metabolite profile of broccoli puree-based on untargeted LC-MS analysis. It demonstrates that fermentation releases bound phytochemicals such as polyphenolic glycosides and glucosinolates and enhances their bioaccessibility.

FIG. 11. Shows a volcano plot indicating metabolites with significant (p<0.05) fold changes after fermentation based on untargeted LC-MS analysis. The top 50 metabolites with significant fold changes and their individual fold changes are recited in Table 8.

FIG. 12. Shows the effect of lactic acid fermentation on broccoli polyphenols based on targeted LC-MS analysis. A 6.6 fold change is observed in chlorogenic acid (2.4 to 15.8 μg/mg), a 23.8 fold increase is observed in sinapic acid (3.6 to 86.6 μg/mg), a 10.5 increase in kaempferol (12.7 to 134.6 μg/mg) and a 0.48 fold decrease is observed in p-coumaric acid.

FIG. 13. Shows the SmaI and NotI restriction enzyme digestion from the genomic DNA of BF1 and BF2 obtained with pulse filed gel electrophoreses.

FIG. 14. Shows that a Brassicaceae product as described herein increases short chain fatty acid production in an in vitro colon fermentation model.

FIG. 15. Shows that a Brassicaceae product as described herein increases short chain fatty acid production in an in vitro colon fermentation model.

FIG. 16. Shows that a Brassicaceae product as described herein increases lactobacilli levels but not E. coli, Bifidobacteria or total bacteria in an in vitro colon fermentation model.

FIG. 17. A) Shows an oxipres trace showing the stability of tuna oil as compared to tuna oil encapsulated in broccoli and broccoli fermented with oil. B) Shows the stability of EPA and DHA encapsulated in non-fermented and fermented broccoli powders during storage at 25° C. C-To-NF, Control broccoli with tuna oil; C-To-F, Control broccoli fermented with tuna oil; Ph-To-NF, Preheated broccoli with tuna oil; Ph-To-F, Preheated broccoli fermented with tuna oil.

### KEY TO THE SEQUENCE LISTING

SEQ ID NO:1—ATCC8014 reference nucleotide sequence position 68529.

SEQ ID NO:2—ATCC8014 reference nucleotide sequence position 72030.

SEQ ID NO:3—ATCC8014 reference nucleotide sequence position 10806.

SEQ ID NO:4—B1 alternate nucleotide sequence position 10806.

SEQ ID NO:5—B1 alternate nucleotide sequence position 50276.

SEQ ID NO:6—ATCC8014 reference nucleotide sequence position 19068.

SEQ ID NO:7—B1 reference nucleotide sequence position 4326.

SEQ ID NO:8—ATCC8293 reference nucleotide sequence position 70144.

SEQ ID NO:9—ATCC8293 reference nucleotide sequence position 341498.

SEQ ID NO:10—BF2 reference nucleotide sequence position 341498.

SEQ ID NO:11—ATCC8293 reference nucleotide sequence position 610344.

SEQ ID NO:12—BF2 reference nucleotide sequence position 610344.

SEQ ID NO:13—ATCC8293 reference nucleotide sequence position 843675.

SEQ ID NO:14—BF2 reference nucleotide sequence position 843675.

SEQ ID NO:15—ATCC8293 reference nucleotide sequence position 986279.

SEQ ID NO:16—BF2 reference nucleotide sequence position 986279.

SEQ ID NO:17—ATCC8293 reference nucleotide sequence position 1319558.

SEQ ID NO:18—BF2 reference nucleotide sequence position 1319558.

SEQ ID NO:19—ATCC8293 reference nucleotide sequence position 1418040.

SEQ ID NO:20—BF2 reference nucleotide sequence position 1418040.

SEQ ID NO:21—ATCC8293 reference nucleotide sequence position 1429917.

SEQ ID NO:22—BF2 reference nucleotide sequence position 1429917.

SEQ ID NO:23—ATCC8293 reference nucleotide sequence position 1430314.

SEQ ID NO:24—BF2 reference nucleotide sequence position 1430314.

SEQ ID NO:25—ATCC8293 reference nucleotide sequence position 1430785.

SEQ ID NO:26—BF2 reference nucleotide sequence position 1430785.

SEQ ID NO:27—ATCC8293 reference nucleotide sequence position 1444575.

SEQ ID NO:28—BF2 reference nucleotide sequence position 1444575.

SEQ ID NO:29—ATCC8293 reference nucleotide sequence position 1629328.

SEQ ID NO:30—BF2 reference nucleotide sequence position 1629328.

SEQ ID NO:31—ATCC8293 reference nucleotide sequence position 1665094.

SEQ ID NO:32—BF2 reference nucleotide sequence position 1665094.

SEQ ID NO:33—ATCC8293 reference nucleotide sequence position 1665337.

SEQ ID NO:34—BF2 reference nucleotide sequence position 1665337.

SEQ ID NO:35—ATCC8293 reference nucleotide sequence position 1696196.

SEQ ID NO:36—BF2 reference nucleotide sequence position 1696196.

SEQ ID NO:37—ATCC8293 reference nucleotide sequence position 1760925.

SEQ ID NO:38—BF2 reference nucleotide sequence position 1760925.

SEQ ID NO:39—ATCC8293 reference nucleotide sequence position 1760994.

SEQ ID NO:40—BF2 reference nucleotide sequence position 1760994.

SEQ ID NO:41—ATCC8293 reference nucleotide sequence position 1761069.

SEQ ID NO:42—BF2 reference nucleotide sequence position 1761069.

SEQ ID NO:43—ATCC8293 reference nucleotide sequence position 1857246.

SEQ ID NO:44—BF2 reference nucleotide sequence position 1857246.

SEQ ID NO:45—ATCC8293 reference nucleotide sequence position 1887567.

SEQ ID NO:46—BF2 reference nucleotide sequence position 1887567.

SEQ ID NO:47—ATCC8293 reference nucleotide sequence position 1887711.

SEQ ID NO:48—BF2 reference nucleotide sequence position 1887711.

SEQ ID NO:49—ATCC8293 reference nucleotide sequence position 1960134.

SEQ ID NO:50—BF2 reference nucleotide sequence position 1960134.

SEQ ID NO:51—ATCC8293 reference nucleotide sequence position 1997007.

SEQ ID NO:52—BF2 reference nucleotide sequence position 1997007.

SEQ ID NO:53—ATCC8293 reference nucleotide sequence position 986375.

SEQ ID NO:54—BF2 reference nucleotide sequence position 986375.

### DETAILED DESCRIPTION

### General Techniques and Definitions

Unless specifically defined otherwise, all technical and scientific terms used herein shall be taken to have the same meaning as commonly understood by one of ordinary skill in the art (e.g., enzyme, fermentation, inoculation).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “about”, unless stated to the contrary, refers to +/−10%, more preferably +/−5%, even more preferably +/−1%, of the designated value.

As used herein, the term “subject” is any animal. In one example, the animal is a vertebrate. For example, the animal is a mammal, avian, arthropod, chordate, amphibian or reptile. Exemplary subjects include but are not limited to human, fish, prawns, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one example, the mammal is a human.

As used herein, the terms “treating” or “treatment” include administering a effective amount of a product, composition or delivery vehicle as described herein sufficient to reduce or eliminate at least one symptom of a specified disease or condition.

As used herein, the terms “prevent” or “preventing” include administering a effective amount of a product, composition or delivery vehicle as described herein sufficient to stop or hinder the development of at least one symptom of a specified disease or condition.

As used herein, “microbiome” refers to the microorganisms in a particular environment which can include the body or a part of the body of a subject. For example, the gut microbiome refers to the community of microorganism in the gut.

As used herein, “microorganism” or “microorganisms” refers to microscopic organisms including bacterial, viral, fungal or eukaryotic organisms.

As used herein “gastrointestinal tract” refers to at least a portion of the gastrointestinal tract. In an embodiment, the portion of the gastrointestinal tract is selected from a portion of the stomach, duodenum, small intestine, large intestine, colon, rectum, cecum, and ileum. In an embodiment, the portion of the gastrointestinal tract is selected from a portion of the small intestine, large intestine and colon.

As used herein “lower gastrointestinal tract” refers to at least a portion of the lower gastrointestinal tract. In an embodiment, the portion of the lower gastrointestinal tract is selected from a portion of the large intestine, cecum, colon and rectum.

As used herein “upper gastrointestinal tract” refers to at least a portion of the upper gastrointestinal tract. In an embodiment, the portion of the upper gastrointestinal tract is selected from a portion of the mouth, pharynx, esophagus, stomach, and duodenum.

### Promoting Health

The present invention provides methods, compositions and delivery vehicles for promoting the health of a subject comprising a Brassicaceae product. As used herein “health” refers to the condition of a subject's body and the extent to which the subjects body is resistant to an illness or free from an illness.

As used herein “promoting health” refers to increasing, enhancing, inducing, and/or stimulating resistance or resilience to an illness or a reduction in one or more symptoms of an illness.

As used herein “resistance” refers to the insensitivity to a disturbance. As used herein “resilience” refers to the rate of the recovery after a disturbance.

In an embodiment, promoting health comprises treating or preventing a condition in a subject.

In an embodiment, promoting health comprises treating or preventing one or more symptoms of a condition selected from: diabetes, inflammation, metabolic dysfunction, allergy and cancer.

In an embodiment, promoting health comprises promoting one or more of: gut health, immune system health, cardiovascular health, central nervous system function, cognition, metabolic health, skeletal health, liver health, blood sugar control and skin health.

In an embodiment, promoting gut health comprises reducing or preventing one or more symptoms of a gut health associated condition selected from one or more of: irritable bowel syndrome, inflammatory bowel disease, Crohn's disease, colorectal cancer, gut leakiness, non-alcoholic fatty liver disease, metabolic syndrome, obesity, small intestinal bacterial overgrowth (SIBO), gastroenteritis, gut microbial dysbiosis, reduced gut microbial diversity, antibiotic treatment, post-surgery recovery, food intolerance, diarrhoea, gastritis, diverticulitis, flatulence, constipation, functional gut disorders and functional gastrointestinal and motility disorders.

In an embodiment, the functional gut disorder is selected from one or more of: functional abdominal bloating/distension, functional constipation, functional diarrhoea, unspecified functional bowel disorder, opioid-induced constipation, centrally mediated abdominal pain syndrome, narcotic bowel syndrome, opioid-induced hyperalgesia, functional pancreatic sphincter of oddi disorder, biliary pain, faecal incontinence, functional anorectal pain, and functional defecation disorders.

In an embodiment, the functional gastrointestinal and motility disorders is selected from one or more of: gastroesophageal reflux disease, intestinal dysmotility, intestinal pseudo-obstruction, small bowel bacterial overgrowth, constipation, outlet obstruction type constipation (pelvic floor dyssynergia), diarrhoea, faecal incontinence, hirschsprung's disease, gastroparesis and achalasia.

In an embodiment, promoting health comprises promoting health of the gut microbiome in a subject. In an embodiment, promoting health of the gut microbiome comprises one or more of: increasing the level and/or activity of one or more beneficial bacteria, decreasing or maintaining the level and/or activity of one or more non-beneficial bacteria, increasing the resistance of the gut microbiome, increasing the resilience of the gut microbiome, and increasing the diversity of the gut microbiome. As used herein “resistance of the gut microbiome” refers to the insensitivity of the gut microbiome to a disturbance. As used herein “resilience of the gut microbiome” refers to the rate of the recovery of the gut microbiome after a disturbance (e.g. a disturbance may reduce the number or type of microorganism in the microbiome).

In an embodiment, the beneficial bacteria is selected from one or more or all of: lactic acid bacteria, Bifidobacteria, Bacteroidetes, Baciullus, Streptococcus, Escherichia and Enterococcus.

In an embodiment, the lactic acid bacteria is selected from one or more of the genera selected from: Lactobacillus, Leuconostoc, Pediococcus, Lactococcus, Streptococcus, Aerococcus, Camobacterium, Enterococcus, Oenococcus, Sporolactobacillus, Tetragenococcus, Vagococcus and Weissella. In an embodiment, the lactic acid bacteria is selected from one or more or all of: Lactobacillus plantarum, Leuconostoc mesenteroides, Lactobacillus rhamnosus, Lactobacillus pentosus, Lactobacillus brevis, Lactococus lactis, Lactobacillus acidophilus, Lactobacillus brevis, Lactobacillus casei, Lactobacillus delbrueckii, Lactobacillus fermentum, Lactobacillus gasseri, Lactobacillus johnsonii, Lactobacillus lactis, Lactobacillus paracasei, Lactobacillus reuteri, Pediococcus pentosaceus and Pedicoccus acidilacti. In an embodiment, the lactic acid bacteria is selected from one or more or all of: i) BF1 deposited under V17/021729 on 25 Sep. 2017 at the National Measurement Institute Australia; ii) BF2 deposited under V17/021730 on 25 Sep. 2017 at the National Measurement Institute Australia; iii) B1 deposited under V17/021731 on 25 Sep. 2017 at the National Measurement Institute Australia; iv) B2 deposited under V17/021732 on 25 Sep. 2017 at the National Measurement Institute Australia; v) B3 deposited under V17/021733 on 25 Sep. 2017 at the National Measurement Institute Australia; vi) B4 deposited under V17/021734 on 25 Sep. 2017 at the National Measurement Institute Australia; and vii) B5 deposited under V17/021735 on 25 Sep. 2017 at the National Measurement Institute Australia.

In embodiment, the Bifidobacteria is selected from one or more of: Bifidobacteria adolescentis, Bifidobacteria animalis, Bifidobacteria bifidum, Bifidobacteria breve, Bifidobacteria infantis, Bifidobacteria longum, and Bifidobacteria thermophilum.

In embodiment, the Baciullus is selected from one or more of: Baciullus cereus, Baciullus clausii, Baciullus coagulans, Baciullus licheniformis, Baciullus pumulis and Baciullus subtilis.

In embodiment, the Streptococcus is Streptococcus thermophiles. In embodiment, the Escherichia is beneficial strain of Escherichia coli.

In embodiment, the Enterococcus is Enterococcus faecium.

In an embodiment, the non-beneficial bacteria is a pathogenic strain of bacteria.

In an embodiment, the non-beneficial bacteria is a pathogenic strain of bacteria selected from one or more of: Escherichia coli, Enterococcus, Helicobacter pylori, Clostridium, Vibrio cholerae, Bacteroides fragilis, Clostridium, Fusobacterium, Staphylococcus (e.g. pneumoniae), Legionella, Haemophilus, Pseudomonas, Prevotella, Salmonella, Campylobacter, and Shigella, Listeria.

In an embodiment, non-beneficial bacteria is a pathogenic strain of Escherichia coli.

In an embodiment, promoting gut health comprises modulating microbial diversity in the gastrointestinal tract of a subject. In an embodiment, modulating microbial diversity comprises increasing microbial diversity. This may occur, for example after a disturbance which reduces the microbial diversity of the gastrointestinal tract.

In an embodiment, promoting gut health comprises treating and/or preventing microbial dysbiosis in the gastrointestinal tract of a subject. As used herein “microbial dysbiosis” refers to an imbalance in the microbiome that is associated with a disease, precedes a disease or occurs as the result of a disease. The imbalance, for example, could be a gain or loss of members of the microbiome community or changes in relative abundance of members of the microbiome community.

In an embodiment, promoting gut health comprises increasing the production of one or more short chain fatty acids including salts or esters thereof (SCFA) in the gastrointestinal tract in the subject. In an embodiment, the production of one or more SCFA is increased in the lower gastrointestinal tract of the subject. In an embodiment, the production of one or more SCFA is increased in the colon of the subject. In an embodiment, the production of one or more SCFA is increased in the subject administered a fermented Brassicaceae product as described herein compared an unfermented Brassicaceae product.

It will be appreciated by persons skilled in the art that production of SCFA in the gastrointestinal tract of a subject can be assessed with standard methods in the research field for measuring SCFA in faecal slurries.

### Prebiotic

In an embodiment, the Brassicaceae product as described herein comprises a prebiotic. As used herein a “prebiotic” refers to a group of nutrients that are degraded by the gut microbiota. Prebiotics result in changes in the composition and/or activity of the gastrointestinal microbiota conferring benefits upon the health of the host (e.g. gut health).

In an embodiment, the prebiotic is converted into one or more SCFA. SCFA positively influence the gastrointestinal microenvironment (increase gut health), in particular the lower gastrointestinal tract including the colon, and distal organ sites as they are small enough to enter the blood and can be delivered to e.g. the central nervous system, immune system and cardiovascular system.

In an embodiment, the prebiotic increases health in the subject by increasing the level of one or more SCFA in the gastrointestinal tract in the subject. In an embodiment, the prebiotic increases the production of one or more SCFA in the gastrointestinal tract in the subject. In an embodiment, the prebiotic increases the production of one or more SCFA in the lower gastrointestinal tract of the subject. In an embodiment, the prebiotic increases the production of one or more SCFA in the colon of the subject.

In an embodiment, the prebiotic increases health in the subject by increasing the level of one or more SCFA in the colon of the subject.

In an embodiment, the SCFA is selected from one or more or all of: butyrate (butanoate), propionate (propanoate), acetate (ethanoate), formate (methanoate), isobutyrate (2-Methylpropanoate), valerate (pentanoate), isovalerate (3-methylbutanoate), caproate (hexanoate), formic acid (methanoic acid), acetic acid (ethanoic acid), propionic acid (propanoic acid), butyric acid (butanoic acid), isobutyric acid (2-methylpropanoic acid), valeric acid (pentanoic acid), isovaleric acid (3-methylbutanoic acid), and caproic acid (hexanoic acid).

In an embodiment, the SCFA is selected from one or more or all of: butyrate, propionate, and acetate. In an embodiment, the SCFA is butyrate. In an embodiment, the SCFA is propionate In an embodiment, the SCFA is acetate.

In an embodiment, the total SCFA level is increased about 30% to about 70% compared to administration of unfermented Brassicaceae. In an embodiment, the total SCFA level is increased about 38% to about 65% compared to administration of unfermented Brassicaceae. In an embodiment, the total SCFA level is increased about 40% to about 60% compared to administration of unfermented Brassicaceae. In an embodiment, the total SCFA level is increased about 40% to about 55% compared to administration of unfermented Brassicaceae.

In an embodiment, the butyrate level is increased about 30% to about 70% compared to administration of unfermented Brassicaceae. In an embodiment, the butyrate is increased about 38% to about 65% compared to administration of unfermented Brassicaceae. In an embodiment, the butyrate level is increased about 40% to about 60% compared to administration of unfermented Brassicaceae. In an embodiment, the butyrate level is increased about 40% to about 55% compared to administration of unfermented Brassicaceae.

In an embodiment, the propionate level is increased about 30% to about 70% compared to administration of unfermented Brassicaceae. In an embodiment, the propionate is increased about 38% to about 65% compared to administration of unfermented Brassicaceae. In an embodiment, the propionate level is increased about 40% to about 60% compared to administration of unfermented Brassicaceae. In an embodiment, the propionate level is increased about 40% to about 55% compared to administration of unfermented Brassicaceae.

In an embodiment, the acetate level is increased about 30% to about 70% compared to administration of unfermented Brassicaceae. In an embodiment, the acetate is increased about 38% to about 65% compared to administration of unfermented Brassicaceae. In an embodiment, the acetate level is increased about 40% to about 60% compared to administration of unfermented Brassicaceae. In an embodiment, the acetate level is increased about 40% to about 55% compared to administration of unfermented Brassicaceae.

In an embodiment, the prebiotic increases the SCFA level about 5 to about 48 hours after administration. In an embodiment, the prebiotic increases the SCFA level about 10 to about 24 hours after administration.

### Probiotic

As used herein a “probiotic” refers to live microorganism which when administered in an adequate amount confers a health benefit to the host (subject). In an embodiment, the Brassicaceae product as described herein comprises a probiotic.

In an embodiment, the probiotic is autochthonous to the Brassicaceae material. In an embodiment, the probiotic is an autochthonous probiotic present on the Brassicaceae material before fermentation. In an embodiment, the probiotic is an allochthonous probiotic added to the Brassicaceae material after fermentation. In an embodiment, the probiotic is the same microorganism used for fermentation. In an embodiment, the probiotic is not active in the fermentation step. In an embodiment, the probiotic is an exogenous probiotic added to the Brassicaceae material before or during fermentation. In an embodiment, the probiotic is an exogenous probiotic added to the Brassicaceae material after fermentation.

In an embodiment, the probiotic is selected from one or more or all of: lactic acid bacteria, Bifidobacteria, Bacteroidetes, Baciullus, Streptococcus, Escherichia, Enterococcus, and Saccharomyces.

In an embodiment, the probiotic is selected from one or more of the genera selected from: Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and Weissella. In an embodiment, the lactic acid bacteria is selected from one or more or all of: Lactobacillus plantarum, Leuconostoc mesenteroides, Lactobacillus rhamnosus, Lactobacillus pentosus, Lactobacillus brevis, Lactococus lactis, Lactobacillus acidophilus, Lactobacillus brevis, Lactobacillus casei, Lactobacillus delbrueckii, Lactobacillus fermentum, Lactobacillus gasseri, Lactobacillus johnsonii, Lactobacillus lactis, Lactobacillus paracasei, Lactobacillus reuteri, Pediococcus pentosaceus and Pedicoccus acidilacti. In an embodiment, the lactic acid bacteria is selected from one or more or all of: i) BF1 deposited under V17/021729 on 25 Sep. 2017 at the National Measurement Institute Australia; ii) BF2 deposited under V17/021730 on 25 Sep. 2017 at the National Measurement Institute Australia; iii) B1 deposited under V17/021731 on 25 Sep. 2017 at the National Measurement Institute Australia; iv) B2 deposited under V17/021732 on 25 Sep. 2017 at the National Measurement Institute Australia; v) B3 deposited under V17/021733 on 25 Sep. 2017 at the National Measurement Institute Australia; vi) B4 deposited under V17/021734 on 25 Sep. 2017 at the National Measurement Institute Australia; and vii) B5 deposited under V17/021735 on 25 Sep. 2017 at the National Measurement Institute Australia.

In embodiment, the Bifidobacteria is selected from one or more of: Bifidobacteria lactis, Bifidobacteria adolescentis, Bifidobacteria animalis, Bifidobacteria bifidum, Bifidobacteria breve, Bifidobacteria infantis, Bifidobacteria longum, and Bifidobacteria thermophilum. In embodiment, the Bifidobacteria is Bifidobacteria animalis. In embodiment, the Bifidobacteria is Bifidobacteria lactis.

In embodiment, the Baciullus is selected from one or more of: Baciullus cereus, Baciullus clausii, Baciullus coagulans, Baciullus licheniformis, Baciullus pumulis and Baciullus subtilis.

In embodiment, the Streptococcus is Streptococcus thermophiles. In embodiment, the Escherichia is beneficial strain of Escherichia coli.

In embodiment, the Enterococcus is Enterociccus faecium.

In embodiment, the Saccharomyces is Saccharomyces cerevisiae.

In an embodiment, the lactic acid bacteria was isolated from a Brassica oleracea. In an embodiment, the lactic acid bacteria was isolated from broccoli. A person skilled in the art will appreciate that this includes direct isolation or indirect isolation (e.g. isolated from an original source and cultivated a number of passages, optionally cryogenically stored, before use). In an embodiment, the lactic acid bacteria was isolated from Australian broccoli. In an embodiment, the lactic acid bacteria is selected from: i) a Leuconostoc mesenteroides; ii) a Lactobacillus plantarum; iii) a Lactobacillus pentosus; iv) a Lactobacillus rhamnosus; v) a combination of i) and ii); vi) a combination of i), ii) and iii); and vii) a combination of i), ii) and iv). In one embodiment, the lactic acid bacteria is selected from one or more or all of BF1, BF2, B1, B2, B3, B4 and B5. In an embodiment, the lactic acid bacteria is B1. In an embodiment, the lactic acid bacteria is B2. In an embodiment, the lactic acid bacteria is B3. In an embodiment, the lactic acid bacteria is B4. In an embodiment, the lactic acid bacteria is B5. In an embodiment, the probiotic is a capsule, tablet, powder or liquid.

In an embodiment, the probiotic is Faecalibacterium prausnitzii. In an embodiment, the probiotic is Akkermansia muciniphila. In an embodiment, the probiotic is microencapsulated as described in WO 2005030229. In an embodiment, the Brassicaceae product as described herein comprises a combined prebiotic and probiotic.

### Synbiotic

In an embodiment, the Brassicaceae product as described herein comprises a prebiotic and a probiotic which are synbiotic. As used herein a “synbiotic” refer to a composition comprising a prebiotic and probiotic which results in a synergistic effect. Synbiotics were developed to overcome possible survival difficulties for probiotics. In an embodiment, the synbiotic improves the shelf life of a live microorganism. In an embodiment, a synbiotic improves the delivery of a live microorganism (e.g. passage of the upper gastrointestinal tract). In an embodiment, a synbiotic improves the survival of live microorganism (e.g. by providing a preferred food source for metabolism by the microorganism). In an embodiment, the composition or delivery vehicle as described herein comprises a prebiotic and a probiotic which are synbiotic

### Brassicaceae

A person skilled in the art will appreciate that the methods as described herein are suitable for producing a fermented product from any Brassicaceae material. As used herein, “Brassicaceae” refers to members of the Family Brassicaceae commonly referred to as mustards, cruicifers or the cabbage family. A person skilled in the art would appreciate that material can be from more than one Brassicaceae.

In an embodiment, the Brassicaceae is selected from the genus Brassica or Cardamine. In an embodiment, the Brassica is selected from Brassica balearica, Brassica carinata, Brassica elongate, Brassica fruticulosa, Brassica hilarionis, Brassica juncea, Brassica napus, Brassica narinosa, Brassica nigra, Brassica oleracea, Brassica perviridis, Brassica rapa, Brassica rupestris, Brassica septiceps, and Brassica tournefortii.

In an embodiment, the Brassica is Brassica oleracea.

In an embodiment, the Brassica is selected from Brassica oleracea variety oleracea (wild cabbage), Brassica oleracea variety capitate (cabbage), Brassica rapa subsp. chinensis (bok Choy), Brassica rapa subsp. pekinensis (napa cabbage), Brassica napobrassica (rutabaga), Brassica rapa var. rapa (turnip), Brassica oleracea variety alboglabra (kai-lan), Brassica oleracea variety viridis (collard greens), Brassica oleracea variety longata (jersey cabbage), Brassica oleracea variety acephala (ornamental kale), Brassica oleracea variety sabellica (kale), Brassica oleracea variety palmifolia (lacinato kale), Brassica oleracea variety ramose (perpetual kale), Brassica oleracea variety medullosa (marrow cabbage), Brassica oleracea variety costata (tronchuda kale), Brassica oleracea variety gemmifera (brussels sprout), Brassica oleracea variety gongylodes (kohlrabi), Brassica oleracea variety italica (broccoli), Brassica oleracea variety botrytis (cauliflower, Romanesco broccoli, broccoli di torbole), Brassica oleracea variety botrytis x italica (broccoflower), and Brassica oleracea variety italica x alboglabra (Broccolini).

In an embodiment, the Brassica is Brassica oleracea, variety italica (broccoli).

In an embodiment, the Brassicaceae is selected from Cardamine hirsuta (bittercress), Iberis sempervirens (candytuft), Sinapis arvensis (charlock), Armoracia rusticana (horseradish), Pringlea antiscorbutica (Kerguelen cabbage), Thlaspi arvense (pennycress), Raphanus raphanistrum subsp. sativus (radish), Eruca sativa (rocket), Anastatica hierochuntica (rose of Jericho), Crambe maritima (sea kale), Cakile maritima (sea rocket), Capsella bursa-pastoris (shepherd's purse), sweet alyssum, Arabidopsis thaliana (thale cress), Nasturtium officinale (watercress), Sinapis alba (white mustard), Erophila verna (whitlow grass), Raphanus raphanistrum (wild radish), Isatis tinctoria (woad), and Nasturtium microphyllum (yellow cress).

In an embodiment, the Brassicaceae has a high level of one or more glucosinolate/s. In an embodiment, the Brassicaceae has been selectively bred to have a high level of one or more glucosinolate/s. In an embodiment, “high level” of a glucosinolate can comprise a higher level of a glucosinolate than shown in Table 2 of Verkerk et al. (2009) in the corresponding Brassicaceae. In an embodiment, a high level of glucosinolate is a level of glucosinolate higher than 3400 μmol/kg dry weight. In an embodiment, a high level of glucosinolate is a level of glucosinolate higher than 4000 mol/kg dry weight. In an embodiment, a high level of glucosinolate is a level of glucosinolate higher than 5000 mol/kg dry weight. In an embodiment, a high level of glucosinolate is a level of glucosinolate higher than 8000 mol/kg dry weight. In an embodiment, a high level of glucosinolate is a level of glucosinolate higher than 10,000 mol/kg dry weight. In an embodiment, a high level of glucosinolate is a level of glucosinolate higher than 12,000 μmol/kg dry weight. In an embodiment, a high level of glucosinolate is a level of glucosinolate higher than 15,000 mol/kg dry weight. In an embodiment, a high level of glucosinolate is a level of glucosinolate higher than 18,000 mol/kg dry weight. In an embodiment, a high level of glucosinolate is a level of glucosinolate higher than 20,000 μmol/kg dry weight. In an embodiment, a high level of glucosinolate is a level of glucosinolate higher than 25,000 mol/kg dry weight. In an embodiment, a high level of glucosinolate is a level of glucosinolate higher than 30,000 mol/kg dry weight. In an embodiment, the Brassicaceae has been genetically modified or subjected to biotic or abiotic stress to have a high level of one or more glucosinolate/s. A person skilled in the art will appreciate that the Brassicaceae can be modified by any method known to a person skilled in the art.

In an embodiment, the glucosinolate is glucoraphanin (4-Methylsulphinylbutyl glucosinolate). In an embodiment, the glucosinolate is glucobrassicin (3-Indolylmethyl glucosinolate).

As used herein “Brassicaceae material” refers to any part of the Brassicaceae, including, but not limited to, the leaves, stems, flowers, florets, seeds, and roots or mixtures thereof.

A person skilled in the art will appreciate that the methods as described herein are suitable for use with different volumes of Brassicaceae material, for example, but not limited to, at least 30 kg, or at least 50 kg, or at least 80 kg, or at least 100 kg, or at least 1,000 kg, or at least 2,000 kg, or at least 5,000 kg, or at least 8,000 kg, or at least 10,000 kg, or at least 15,000 kg, or at least 20,000 kg.

In an embodiment, the Brassicaceae material has been washed. As used herein “washing” removes visible soil and contamination. In an embodiment, the Brassicaceae material has been sanitized. As used herein “sanitized” refers to a reduction of pathogens on the Brassicaceae material.

In an embodiment, the Brassicaceae is mixed with other plant material. In an embodiment, the other plant material is vegetable or fruit material. In an embodiment, the vegetable is a carrot or beetroot.

### Pre-Treatment

As use herein “pre-treatment” or “pre-treating” the Brassicaceae material increases the bioavailability of one or more components in the Brassicaceae material.

In an embodiment, the component is fibre. In an embodiment, the component is a prebiotic and/or prebiotic precursor. In an embodiment, the prebiotic is selected from one or more or all of: dietary fibre (insoluble/soluble), oligosaccharides, cellulose, hemicellulose, pecticoligosaccharide, resistant starch beta-glucans and pectin.

In an embodiment, the component is an antimicrobial component. In an embodiment, the antimicrobial component is a glucosinolate.

In an embodiment, the component is a bioactive peptide. In an embodiment, the peptide has angiotensin-converting-enzyme inhibitory activity.

In an embodiment, the component is a polyphenol. As used herein, “polyphenol” refers to a compound comprising more than one phenolic hydroxyl group. In an embodiment, the polyphenol is selected from one or more of: anthocyanins, dihydrochalcones, flavan-3-ols, flavanones, flavones, flavonols and isoflavones, curcumin, resveratrol, benzoic acid, phenyl acetic acid, hydroxycinnamic acids, coumarins, napthoquinones, xanthones, stilbenes, chalcones, tannins, phenolic acids, and catechins (e.g. epigallocatechin gallate (EGCg), epigallocatechin (EGC), epicatechin gallate (ECg), epicatechin (EC), and their geometric isomers gallocatechin gallate (GCg), gallocatechin (GC), catechin gallate (Cg) and catechin.

In an embodiment, pre-treating alters the activity of one or more indigenous plant enzymes (eg cell-wall degrading enzymes such as pectinase, xylanases, cellulases), with consequent effects of nutritional properties of fibre and the accessibility of plant bioactives.

In an embodiment, pre-treating comprises one or more of the following: i) heating; ii) macerating; iii) microwaving; iv) exposure to high frequency sound waves (ultrasound), v) pulse electric field processing and vi) high pressure processing. In an embodiment, the temperature of the Brassicaceae material does not exceed about 75° C. during pre-treating.

In an embodiment, the Brassicaceae material is heated in a fuel based heating system, an electricity based heating system (i.e. an oven or ohmic heating), radio frequency heating, high pressure thermal processing or a steam based heating system (indirect or direct application of steam). In an embodiment, the Brassicaceae material is heated in a sealed package (e.g. in a retort pouch). In an embodiment, the Brassicaceae material is heated in an oven, water bath, bioreactor, stove, water blancher, or steam blancher. In an embodiment, the Brassicaceae material is heated via high pressure thermal heating. In an embodiment, the Brassicaceae material is via ohmic heating. In an embodiment, the Brassicaceae material is via radio frequency heating. In an embodiment, the Brassicaceae material is blanched in water. In an embodiment, the Brassicaceae material is heated via high pressure thermal processing. In an embodiment, the Brassicaceae material is placed in a sealed package for high pressure thermal processing.

In an embodiment, pre-treating comprises heating the Brassicaceae material to about 50° C. to about 70° C. In an embodiment, pre-treating comprises heating the Brassicaceae material to about 50° C. to about 65° C. In an embodiment, pre-treating comprises heating the Brassicaceae material to about 50° C. to about 60° C. In an embodiment, heating comprises heating the Brassicaceae material to about 55° C. to about 70° C. In an embodiment, heating comprises heating the Brassicaceae material to about 60° C. to about 70° C. In an embodiment, heating comprises heating the Brassicaceae material to about 65° C. to about 70° C. In an embodiment, the Brassicaceae material is heated for about 30 seconds. In an embodiment, the Brassicaceae material is heated for about 1 minute. In an embodiment, the Brassicaceae material is heated for about 2 minutes. In an embodiment, the Brassicaceae material is heated for about 3 minutes. In an embodiment, the Brassicaceae material is heated for about 4 minutes. In an embodiment, the Brassicaceae material is heated for about 5 minutes.

In an embodiment, the Brassicaceae material is heated in a sealed package for about 1 min at about 60° C. In an embodiment, the Brassicaceae material is heated in a sealed package for about 2 mins at about 60° C. In an embodiment, the Brassicaceae material is heated in a sealed package for about 3 mins at about 60° C. In an embodiment, the Brassicaceae material is heated in a sealed package for about 4 mins at about 65° C. In an embodiment, the Brassicaceae material is heated in a sealed package for about 1 min at about 65° C. In an embodiment, the Brassicaceae material is heated in a sealed package for about 2 mins at about 65° C. In an embodiment, the Brassicaceae material is heated in a sealed package for about 3 mins at about 65° C. In an embodiment, the Brassicaceae material is heated in a sealed package for about 4 mins at about 65° C.

In an embodiment, the Brassicaceae material is heated in water for about 1 min at about 60° C. In an embodiment, the Brassicaceae material is heated in water for about 2 mins at about 60° C.

In an embodiment, heating comprises steaming the Brassicaceae material. In an embodiment, pre-treating comprises steaming the Brassicaceae material. In an embodiment, the Brassicaceae material is steamed to a temperature of about 50° C. to about 70° C. In an embodiment, the Brassicaceae material is steamed to a temperature of about 60° C. to about 70° C. In an embodiment, the Brassicaceae material is steamed for at least about 30 seconds. In an embodiment, the Brassicaceae material is steamed for at least about 1 minute. In an embodiment, the Brassicaceae material is steamed for at least about 2 minutes. In an embodiment, the Brassicaceae material is steamed for at least about 3 minutes. In an embodiment, the Brassicaceae material is steamed for at least about 4 minutes. In an embodiment, the Brassicaceae material is steamed for at least about 5 minutes.

In an embodiment, pre-treating comprises macerating the Brassicaceae material. As used herein “macerating”, “macerated” or “macerate” refers to breaking the Brassicaceae material into smaller pieces. In an embodiment, macerating comprising decompartmentalizing at least about 30% to about 90% of the cells of the Brassicaceae material to allow myrosinase access to its substrate glucosinolates. In an embodiment, macerating comprising decompartmentalizing at least about 40% to about 90% of the cells of the Brassicaceae material. In an embodiment, macerating comprising decompartmentalizing at least about 50% to about 90% of the cells of the Brassicaceae material. In an embodiment, macerating comprising decompartmentalizing at least about 60% to about 90% of the cells of the Brassicaceae material. In an embodiment, macerating comprising decompartmentalizing at least about 70% to about 90% of the cells of the Brassicaceae material. A person skilled in the art will appreciate that decompartimentalizing a cell comprising breaking open the cell wall and disrupting the compartmentalization of organelles within a cell.

In an embodiment, the Brassicaceae material is macerated with a blender, grinder or pulveriser. In an embodiment, the Brassicaceae material is macerated so that at least about 80% of the Brassicaceae material is of a size of about 2 mm or less. In an embodiment, the Brassicaceae material is macerated so that at least about 80% of the Brassicaceae material is of a size of about 1 mm or less. In an embodiment, the Brassicaceae material is macerated so that at least about 80% of the Brassicaceae material is of a size of about 0.5 mm or less. In an embodiment, the Brassicaceae material is macerated so that at least about 80% of the Brassicaceae material is of a size of about 0.25 mm or less. In an embodiment, the Brassicaceae material is macerated so that at least about 80% of the Brassicaceae material is of a size of about 0.1 mm or less. In an embodiment, the Brassicaceae material is macerated so that at least about 80% of the Brassicaceae material is of a size of about 0.05 mm or less. In an embodiment, the Brassicaceae material is macerated so that at least about 80% of the Brassicaceae material is of a size of about 0.025 mm or less. In an embodiment, the Brassicaceae material is macerated so that at least about 80% of the Brassicaceae material is of a size of about 0.01 mm or less. In an embodiment, the Brassicaceae material is macerated so that about 50% to about 90% of the Brassicaceae material is of a size of about 2 mm or less. In an embodiment, the Brassicaceae material is macerated so that about 60% to about 80% of the Brassicaceae material is of a size of about 2 mm or less. In an embodiment, the Brassicaceae material is macerated so that about 50% to about 90% of the Brassicaceae material is of a size of about 1 mm or less. In an embodiment, the Brassicaceae material is macerated so that about 60% to about 80% of the Brassicaceae material is of a size of about 1 mm or less. In an embodiment, the Brassicaceae material is heated to a temperature of about 50° C. to about 70° C. during maceration. In an embodiment, the Brassicaceae material is heated to a temperature of about 55° C. to about 70° C. during maceration. In an embodiment, the Brassicaceae material is heated to a temperature of about 60° C. to about 70° C. during maceration. In an embodiment, the Brassicaceae material is heated to a temperature of about 65° C. to about 70° C. during maceration.

In an embodiment, pre-treating comprises heating and macerating the Brassicaceae material. In an embodiment, pre-treating produces a puree. As used herein a “puree” refers to Brassicaceae material blended to the consistency of a creamy paste or liquid.

A person skilled in the art will appreciate that “microwaves” or “microwaving” heats a substance such as Brassicaceae material by passing microwave radiation through the substance. In an embodiment, pre-treating comprises microwaving the Brassicaceae material. In an embodiment, Brassicaceae material is pre-treated in a consumer microwave or industrial microwave. In an embodiment, the industrial microwave is a continuous microwave system, for example, but not limited to the MIP 11 Industrial Microwave Continuous Cooking Over (Ferrite Microwave Technologies). In an embodiment, pre-treating comprises microwaving the Brassicaceae material. In an embodiment, the Brassicaceae material is microwaved at about 0.9 to about 2.45 GHz. In an embodiment, the Brassicaceae material is microwaved for at least about 30 seconds, or at least about 1 minute, or at least about 2 minutes, or at least 3 minutes.

In an embodiment, pre-treating comprises exposing the Brassicaceae material at low to medium frequency ultrasound waves. In an embodiment, pre-treating comprises exposing the Brassicaceae material with thermosonication (low to medium frequency ultrasound waves with heat of about 30° C. to about 60° C.). In an embodiment, the ultrasound waves are generated with an industrial scale ultrasonic processor. In an embodiment, the ultrasonic processor is a continuous or batch ultrasonic processor. In an embodiment, the ultrasonic processor is for example, but not limited to, UIP500hd or UIP4000 (Hielscher, Ultrasound Technology). In an embodiment, the ultrasounds waves are at a frequency of about 20 kHz to about 600 kHz. In an embodiment, the Brassicaceae material is exposed to sound waves for at least about 30 seconds, or at least about 1 minute, or at least about 2 minutes, or at least about 3 minutes, or about 5 minutes.

In an embodiment, pre-treating comprises exposing the Brassicaceae material to pulse electric field processing. Pulse electric field processing is a non-thermal processing technique comprising the application of short, high voltage pulses. The pulses induce electroporation of the cells of the Brassicaceae material enhancing the access of myrosinase to glucosinolates. In an embodiment, pulse electric field processing heats the Brassicaceae material to a temperature of about 40 to about 70° C. In an embodiment, pulse electric field processing heats the Brassicaceae material to a temperature of about 50° C. to about 70° C. In an embodiment, pulse electric field processing heats the Brassicaceae material to a temperature of about 60° C. to about 70° C. In an embodiment, pulse electric field processing comprises treating the Brassicaceae material with voltage pulses of about 20 to about 80 kV.

In an embodiment, pre-treating comprises exposing the Brassicaceae material to high pressure processing. In an embodiment, the Brassicaceae product is in a sealed package during high pressure processing. In an embodiment, high pressure processing comprises treating the Brassicaceae material with isostatic pressure at about 100 to about 800 MPa. In an embodiment, high pressure processing comprises treating the Brassicaceae material with isostatic pressure at about 100 to about 600 MPa. In an embodiment, high pressure processing comprises treating the Brassicaceae product with isostatic pressure at about 350 to about 550 MPa. In an embodiment, high pressure processing comprises treating the Brassicaceae product with isostatic pressure at about 300 to about 400 MPa. In an embodiment, heat treatment comprises heating the sample to a temperature of about 60° C. to about 121° C. In an embodiment, heat treatment comprises heating the sample to a temperature of about 65° C. to about 100° C. In an embodiment, heat treatment comprises heating the sample to a temperature of about 65° C. to about 80° C. In an embodiment, heat treatment comprises heating the sample to a temperature of about 65° C. to about 75° C.

In an embodiment, pre-treating converts about 10% to about 90% of a glucosinolate to an isothiocyanate. In an embodiment, pre-treating converts about 20% to about 80% of a glucosinolate to an isothiocyanate. In an embodiment, pre-treating converts about 30% to about 70% of a glucosinolate to an isothiocyanate.

### Preparation of an Emulsion or Suspension

In an aspect, the present invention provides a method of preparing a Brassicaceae product comprising:

i) fermenting Brassicaceae material with lactic acid bacteria;

ii) adding a fatty acid and/or oil before or during step i).

In an embodiment, the method further comprises forming an emulsion or suspension.

In an embodiment, the Brassicaceae material is pre-treated as described herein. In an embodiment, the Brassicaceae material is pre-treated by heating as described herein. In an embodiment, the Brassicaceae material is heated to about 50° C. to about 70° C.

In an embodiment, the fatty acid and/or oil is added before step i). In an embodiment, the fatty acid and/or oil is added during step i). In an embodiment, the fatty acid and/or oil is added before pre-treatment. In an embodiment, the fatty acid and/or oil is added during pre-treatement. In an embodiment, the fatty acid and/or oil is added after pre-treatment. In an embodiment, the fatty acid and/or oil is added after pre-treatment and before step i).

In an aspect, the present invention provides an emulsion or suspension produced by the method as described herein.

In an aspect, the present invention provides a Brassicaceae product comprising the emulsion or suspension as described herein.

As used herein “emulsion” refers to a dispersion of droplets/particles of one liquid in another in which it is not soluble or miscible. In one embodiment, the droplets are fatty acid and/or oil dispersed in the aqueous mixture. In an embodiment, the emulsion is a wet emulsion. In an embodiment, the emulsion is dried into powder. In an embodiment, the emulsion is extruded. In an embodiment, the emulsion is extruded with a powder matrix.

In an embodiment, droplets produced by the methods described herein are about 0.2 μm to about 10 μm. In an embodiment, droplets produced by the methods described herein are about 1 μm to about 10 μm. In an embodiment, droplets produced by the methods described herein are about 2 μm to about 8 μm. In an embodiment, droplets produced by the methods described herein are about 2 μm to about 4 μm.

In an embodiment, the mean droplet size is about 0.2 μm to about 10 μm. In an embodiment, the mean droplet size is about 1 μm to about 10 μm. In an embodiment, the mean droplet size is about 2 μm to about 8 μm. In an embodiment, the mean droplet size is about 2 μm to about 4 μm.

As used herein “suspension” refers to dispersion of droplets/particles of one substance throughout the bulk of another substance. In one embodiment, the droplets are a fatty acid and/or oil dispersed in the aqueous mixture.

As used herein producing or forming an emulsion or suspension refers to entrapment or encapsulation of a substance in the aqueous mixture reducing the exposure of the substance to degradation. In an embodiment, the substance is a fatty acid and/or oil.

In an embodiment, the fatty acid and/or oil is heated when it is added to the aqueous mixture in step ii) as described herein. In an embodiment, the fatty acid and/or oil is heated to about 30° C. to about 80° C. In an embodiment, the fatty acid and/or oil is heated to about 40° C. to about 70° C. In an embodiment, the fatty acid and/or oil is heated to about 45° C. to about 65° C. In an embodiment, the fatty acid and/or oil is heated to about 50° C. to about 60° C.

In an embodiment, forming an emulsion or suspension as described comprises mixing of the fatty acid and/oil with an aqueous mixture comprising the Brassicaceae material.

In an embodiment, mixing comprises agitation under high shear. In an embodiment, mixing comprises homogenization to obtain a small droplet size. In an embodiment, droplets produced by homogenization are about 0.2 μm to about 10 μm in diameter. In an embodiment, droplets produced by homogenization are about 1 μm to about 10 μm in diameter. In an embodiment, droplets produced by homogenization are about 2 μm to about 8 μm in diameter. In an embodiment, droplets produced by homogenization are about 2 μm to about 4 μm in diameter. In an embodiment, homogenization forms a homogenous emulsion.

As used herein, the term “fatty acid” refers to a carboxylic acid (or organic acid), often with a long aliphatic tail, either saturated or unsaturated. Typically fatty acids have a carbon-carbon bonded chain of at least 4 carbon atoms (C4) or at least 8 carbon atoms (C8) in length, more preferably at least 12 carbons in length. Preferred fatty acids of the invention have carbon chains of 18-22 carbon atoms (C18, C20, C22 fatty acids), more preferably 20-22 carbon atoms (C20, C22) and most preferably 22 carbon atoms (C22). Most naturally occurring fatty acids have an even number of carbon atoms because their biosynthesis involves acetate which has two carbon atoms. The fatty acids may be in a free state (non-esterified) or in an esterified form such as part of a triglyceride, diacylglyceride, monoacylglyceride, acyl-CoA (thio-ester) bound or other bound form. The fatty acid may be esterified as a phospholipid such as a phosphatidylcholine, phosphatidylethanolamine, phosphatidylserine, phosphatidylglycerol, phosphatidylinositol or diphosphatidylglycerol forms. In an embodiment, the fatty acid is esterified to a methyl or ethyl group, such as, for example, a methyl or ethyl ester of a C20 or C22 polyunsaturated fatty acid. Preferred fatty acids are the methyl or ethyl esters of eicosatrienoic acid, docosapentaenoic acid or docosahexaenoic acid, or the mixtures eicosapentaenoic acid and docosahexaenoic acid, or eicosapentaenoic acid, docosapentaenoic acid and docosahexaenoic acid, or eicosapentaenoic acid and docosapentaenoic acid.

In an embodiment, the fatty acid is a polyunsaturated fatty acid. As used herein “polyunsaturated fatty acid” refers to a fatty acid that contains more than one double bond in its backbone. In an embodiment, the polyunsaturated fatty acid is selected from one or more of: an omega-3, omega-6, or omega-9. In an embodiment, the polyunsaturated fatty acid is an omega-3. In an embodiment, the polyunsaturated fatty acid is an omega-6. In an embodiment, the polyunsaturated fatty acid is an omega-9. In an embodiment, the omega-3 is selected from one or more of: hexadecatrienoic acid, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, and tetracosahexaenoic acid. In an embodiment, the omega-3 is selected from one or more or all of eicosapentaenoic acid, docosapentaenoic acid and docosahexaenoic acid. In an embodiment, the omega-6 is selected from one or more of: linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, and tetracosapentaenoic acid. In an embodiment, the omega-9 is selected from one or more of: oleic acid, eicosenoic acid, mead acid, erucic acid, and nervonic acid.

In an embodiment, the fatty acid is in an oil.

As used herein “oil” refers to a viscous liquid that is hydrophobic and lipophilic and not miscible with water.

In an embodiment, the oil is an unsaturated oil.

In an embodiment, the oil is a Plantae oil. In an embodiment, the oil is a vegetable oil. In an embodiment, the oil is an animal oil. In an embodiment, the animal oil is a marine oil or fish oil.

In an embodiment, the oil is selected from one or more of: fish oil, krill oil, marine oil, algal oil, microbial oil, canola oil, crustacean oil, mollusc oil, sunflower oil, avocado oil, soya oil, borage oil, evening primrose oil, safflower oil, flaxseed oil, olive oil, pumpkinseed oil, hemp seed oil, wheat germ oil, palm oil, palm oil, palm kernel oil, coconut oil, medium chain triglycerides (MCT) and grapeseed oil. In an embodiment, the canola oil comprises one or more long chain polyunsaturated fatty acids such as eicosapentaenoic acid (EPA), docosapentaenoic acid (DPA) and docosahexaenoic acid (DHA) which can be obtained from transgenic Brassica encoding the required elongases and desaturases (see, for example, WO 2015/089587).

In an embodiment, the fish oil is selected from one or more of: tuna oil, herring oil, mackerel oil, anchovy oil, sardine oil, cod liver oil, and shark oil.

As used herein “microbial oil” also known as “single cell oil” is an oil produced by a micorbe. For example, the microbe is a yeast, fungus, microalgae or bacteria. In an embodiment, yeast is selected from one or more of: R. toruloides 32489, R. toruloides ATCC 10788, Cryptococcus curvatus, Candida curvata, Cryptococcus albidus, Lipomyces starkeyi and Rhodotorula glutinis. In an embodiment, the fungus is selected from one or more of: Aspergillus oryzae, Mortierella isabellina, and Humicola lanuginose. In an embodiment, the microalge is selected from one or more of: Botryococcus braunii, Mucor circinelloides, Aspergillus niger, Cylindrotheca sp., Chlorella sp., Nitzschia sp., Schizochytrium sp., Crypthecodinium cohnii, Nannochloropsis sp., Neochloris oleoabundans, and Nannochloris sp. In an embodiment, the bacteria is selected from one or more of: Arthrobacter sp., Acinetobacter calcoaceticus and Rhodococcus opacus.

In an embodiment, the mollusc is abalone.

In an embodiment, the essential oil is selected from one or more of: oregano oil, mint oil, basil oil, rosemary oil, tea tree oil, time oil, camphor oil, cardamon oil, citrus oil, clove oil, and/or saffron oil.

In an embodiment, the oil comprises dairy fats.

In an embodiment, the oil is olive oil.

In an embodiment, the oil is sunflower oil.

In an embodiment, the oil is canola oil.

In an embodiment, the oil comprises one or more bioactive/s and/or bioactive precursor/s. Thus, in some embodiments, the oil acts as a bioactive carrier. In an embodiment, the bioactive and/or bioactive precursor is added to the oil before the oil is added to the aqueous mixture. In an embodiment, the bioactive and/or bioactive precursor is infused in oil in step ii) of the method as described herein. In an embodiment, the bioactive and/or a bioactive precursor is infused in oil in step iii) of the method as described herein. In an embodiment, the bioactive and/or bioactive precursor is from the biomass and/or further biomass as described herein. In an embodiment, the bioactive and/or bioactive precursor is not from the biomass and/or further biomass.

### Fermentation

Brassicaceae material, optionally pre-treated, is fermented as described herein to produce a fermented Brassicaceae product. In an embodiment, the Brassicaceae material is optionally mixed with a fatty acid and/or oil before fermentation. As used herein, “fermentation” refers to the biochemical breakdown of the Brassicaceae material by lactic acid bacteria. In an embodiment, fermentation with lactic acid bacteria is performed using the addition of exogenous lactic acid bacteria. In an embodiment, fermentation increases the quantity and bioavailability of one or more components in the Brassicaceae material. In an embodiment, the component is a prebiotic and/or prebiotic precursor. dietary fibre (insoluble/soluble), oligosaccharides, cellulose, hemicellulose,

In an embodiment, the prebiotic is selected from one or more or all of: dietary fibre, oligosaccharides, exopolysaccharides, oligofructose, cellulose, hemicellulose resistant starch, beta-glucans and dextran. In an embodiment, the oligosaccharides are selected from one or more or all of: gluco-oligosaccharides fructo-oligosaccharides galacto-oligosaccharide, trans-galacto-oligosaccharides. In an embodiment, the exopolysaccharides are homopolysaccharides and/or heteropolysaccharides.

In an embodiment, the component is a bioactive peptide. In an embodiment, the peptide is an antimicrobial peptide (e.g. bacteriocins or those described in Pacheco-Cano et al., 2017). In an embodiment, the peptide has angiotensin-converting-enzyme inhibitory activity.

As used herein, “lactic bacteria” or “lactic acid bacteria” are bacteria that produce lactic acid as an end product of carbohydrate fermentation, and can include, but are not limited to including bacteria from the genera Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and Weissella. In an embodiment, the lactic acid bacteria comprises myrosinase activity. In an embodiment, the lactic acid bacteria is from the genera Leuconostoc. In an embodiment, the lactic acid bacteria is from the genera Lactobacillus.

In an embodiment, the lactic acid bacteria is selected from one or more of Lactobacillus plantarum, Leuconostoc mesenteroides, Lactobacillus rhamnosus, Lactobacillus pentosus, Lactobacillus brevis, Lactococus lactis, Pediococcus pentosaceus and Pedicoccus acidilacti.

In an embodiment, the lactic acid bacteria were derived from an isolate obtained from Brassicaceae. In an embodiment, the Brassicaceae material has been pre-treated as described herein. In an embodiment, the lactic acid bacteria was derived from an isolate obtained from Brassica oleracea. In an embodiment, the lactic acid bacteria was derived from an isolate obtained from broccoli. As used herein “derived from” means isolated directly from or indirectly from a culture derived from an indicated source which has subsequently been passaged in culture (e.g. an isolate initially isolated from broccoli which has subsequently been passaged a number of times in in vitro cell culture). In an embodiment, the lactic acid bacteria was isolated from broccoli leaves. In an embodiment, the lactic acid bacteria was isolated from broccoli stem. In an embodiment, the lactic acid bacteria was isolated from broccoli puree. In an embodiment, the lactic acid bacteria was isolated from Australian broccoli.

In an embodiment, the lactic acid bacteria lacks myrosinase activity.

In an embodiment, the lactic acid bacteria is a Lactobacillus.

In an embodiment, the lactic acid bacteria is selected from: i) a Leuconostoc mesenteroides; ii) a Lactobacillus plantarum; iii) a Lactobacillus pentosus; iv) a Lactobacillus rhamnosus; v) a combination of i) and ii); vi) a combination of i), ii) and iii); and vii) a combination of i), ii) and iv).

In one embodiment, the lactic acid bacteria is Leuconostoc mesenteroides. In an embodiment, the Leuconostoc mesenteroides is ATCC8293. In an embodiment, the Leuconostoc mesenteroides is BF1 and/or BF2. In an embodiment, the Leuconostoc mesenteroides lacks myrosinase activity.

In one embodiment, the lactic acid bacteria is Lactobacillus plantarum. In an embodiment, the Lactobacillus plantarum lacks myrosinase activity.

In one embodiment, about 50% of the lactic acid bacteria is Leuconostoc mesenteroides and about 50% of the lactic acid bacteria is Lactobacillus sp.

In one embodiment, about 50% of the lactic acid bacteria is Leuconostoc mesenteroides and about 50% of the lactic acid bacteria is Lactobacillus plantarum. In an embodiment, the Lactobacillus plantarum is selected from one or more or all of B1, B2, B3, B4 and B5. In an embodiment, the Lactobacillus plantarum is B1. In an embodiment, the Lactobacillus plantarum is B2. In an embodiment, the Lactobacillus plantarum is B3. In an embodiment, the Lactobacillus plantarum is B4. In an embodiment, the Lactobacillus plantarum is B5.

In an embodiment, fermentation occurs in the presence of at least 2, or at least 3, or at least 4, or at least 5, or at least 6 strains of lactic acid bacteria selected from BF1, BF2, B1, B2, B3, B4 and B5.

In one embodiment, the lactic acid bacteria is a recombinant bacteria modified to produce a high level of myrosinase activity compared to a control bacteria lacking the modification. A person skilled in the art will appreciate that the recombinant lactic acid bacteria is produced by any technique known to a person skilled in the art.

In an embodiment, the lactic acid bacteria is stressed, for example but not limited to, heat stress, cold stress, sub-lethal ultrasonic waves e.g. about 20 to about 2000 MHz, high pressure, dynamic high pressure or pulsed-electric field, to increase myrosinase activity and the activity of polysaccharide degrading enzymes compared to a control lactic acid bacteria that has not been stressed.

In an embodiment, the Brassicaceae material is inoculated with at least about 10⁵ CFU/g of a lactic acid bacteria as described herein. In an embodiment, the Brassicaceae material is inoculated with at least 10⁶ about CFU/g of a lactic acid bacteria as described herein. In an embodiment, the Brassicaceae material is inoculated with at least about 10⁷ CFU/g of a lactic acid bacteria as described herein. In an embodiment, the Brassicaceae material is inoculated with at least about 10⁸ CFU/g of a lactic acid bacteria as described herein. In an embodiment, the Brassicaceae material has been pre-treated.

In an embodiment, fermentation is at about 20° C. to about 34° C. In an embodiment, fermentation is at about 22° C. to about 34° C. In an embodiment, fermentation is at about 24° C. to about 34° C. In an embodiment, fermentation is at about 24° C. to about 30° C. In an embodiment, fermentation is at about 34° C. to about 34° C. In an embodiment, fermentation is at about 25° C. In an embodiment, fermentation is at about 30° C. In an embodiment, fermentation is at about 34° C.

In an embodiment, fermentation is for about 8 hours to about 17 days. In an embodiment, fermentation is for about 8 hours to about 14 days. In an embodiment, fermentation is for about 8 hours to about 7 days. In an embodiment, fermentation is for about 8 hours to about 5 days. In an embodiment, fermentation is for about 8 hours to about 4 days. In an embodiment, fermentation is for about 8 hours to about 3 days. In an embodiment, fermentation is for about 8 hours to about 30 hours. In an embodiment, fermentation is for about 8 to about 24 hours. In an embodiment, fermentation is for about 10 hours to about 24 hours. In an embodiment, fermentation is for about 10 days. In an embodiment, fermentation is for about 9 days. In an embodiment, fermentation is for about 8 days. In an embodiment, fermentation is for about 7 days. In an embodiment, fermentation is for about 4 days. In an embodiment, fermentation is for about 6 days. In an embodiment, fermentation is for about 5 days. In an embodiment, fermentation is for about 72 hours. In an embodiment, fermentation is for about 60 hours. In an embodiment, fermentation is for about 45 hours. In an embodiment, fermentation is for about 30 hours. In an embodiment, fermentation is for about 24 hours. In an embodiment, fermentation is for about 20 hours. In an embodiment, fermentation is for about 18 hours. In an embodiment, fermentation is for about 15 hours. In an embodiment, fermentation is for about 16 hours. In an embodiment, fermentation is for about 14 hours. In an embodiment, fermentation is for about 12 hours. In an embodiment, fermentation is for about 10 hours. In an embodiment, fermentation is for about 8 hours. In an embodiment, the fermentation culture is stirred. In an embodiment, stirring is intermittent. In an embodiment, stirring is continuous. In a particularly preferred embodiment, fermentation is for 15 hours with intermittent stirring. In a particularly preferred embodiment, fermentation is for 24 hours with intermittent stirring.

In an embodiment, the fermentation reaction is complete when the composition reaches a pH of about 4.5 to about 3.8. In an embodiment, the fermentation reaction is complete when the composition reaches a pH of about 4.5 to about 3.6. In an embodiment, the fermentation reaction is complete when the composition reaches a pH of about 4.5 to about 4.04. In an embodiment, the fermentation reaction is complete when the composition reaches a pH of about 4.3 to about 4.04. In an embodiment, the fermentation reaction is complete when the composition reaches a pH of 4.5 or less, or 4.4 or less, or 4.3 or less, or 4.04 or less, or 3.8 or less. In an embodiment, the fermentation reaction is complete when the composition reaches a pH of 4.5 or less. In an embodiment, the fermentation reaction is complete when the composition reaches a pH of 4.4 or less.

In an embodiment, if present fermentation reduces the number of one or more or all of: E. coli, Salmonella and Listeria. In an embodiment, if present fermentation reduces the CFU/g of one or more or all of: E. coli, Salmonella and Listeria.

In an embodiment, no salt is added to the fermentation culture.

In an embodiment, fermentation increases the extractable glucosinolate content compared to the extractable glucosinolate content in the pre-treated Brassicaceae material. In an embodiment, fermentation increases the extractable glucosinolate content compared to the extractable glucosinolate content in the Brassicaceae material. In an embodiment, fermentation increases the extractable glucosinolate content is increased by about 100% to about 500% compared to the extractable glucosinolate content in the Brassicaceae material. In an embodiment, fermentation increases the extractable glucosinolate content by about 200% to about 450% compared to the extractable glucosinolate content in the Brassicaceae material. In an embodiment, fermentation increases the extractable glucosinolate content by about 250% to about 450% compared to the extractable glucosinolate content in the Brassicaceae material. In an embodiment, fermentation increases the extractable glucosinolate content by about 300% to about 400% compared to the extractable glucosinolate content in the Brassicaceae material. In an embodiment, fermentation increases the extractable glucosinolate content by about 300% compared to the extractable glucosinolate content in the Brassicaceae material. In an embodiment, fermentation increases the extractable glucosinolate content by about 400% compared to the extractable glucosinolate content in the Brassicaceae material. In an embodiment, the glucosinolate is glucoraphanin

### Acidification

The pre-treated material can by acidified to improve the microbial safety and stability (susceptibility to microbial degradation) of the product. Acidification can be achieved by the addition of organic acids, such as, but not limited to lactic, acetic, ascorbic, and citric acid. In embodiment, acidification can be achieved with the addition of glucono-delta-lactone. In an embodiment, acidification comprises lowering the pH to a pH of about 4.4 to about 3.4. In an embodiment, acidification comprises lowering the pH to a pH of 4.5, or 4.4, or 4.2, or 4, or 3.8, or 3.6, or 3.4 or less. In an embodiment, acidification comprises lowering the pH to a pH of 4.4 of less.

### Post-Treatment

In an embodiment, after fermentation or acidification the Brassicaceae product can be post-treated to inactivate microbes that for example contribute to degradation of the product or a pathogenic if consumed.

As used herein “post-treatment” or “post-treating” refers to treatment of the Brassicaceae product after fermentation. As used herein “microbes” refers to bacterial, viral, fungal or eukaryotic activity that can result in degradation or spoilage of the Brassicaceae product. As used herein “inactivate” or “inactivation” of microbes refers to reducing the viable microbes by about 1 to about 7 logs. In an embodiment, the viable microbes are reduced by about 1 to 6 logs. In an embodiment, the viable microbes are reduced by about 2 to 6 logs. In an embodiment, the viable microbes are reduced by about 3 to 6 logs.

A person skilled in the art will appreciate that the post treatment can be any method that inactivates microbes, including for example, heat treatment, UV treatment, ultrasonic processing, pulsed electric field processing or high pressure processing. In an embodiment, the Brassicaceae product is post-treated with heat processing. In an embodiment, the Brassicaceae product is post-treated with high pressure processing. In an embodiment, the Brassicaceae product is in a sealed package during post-treatment. In an embodiment, the Brassicaceae product is in a sealed package during high pressure processing. In an embodiment, the Brassicaceae product is in a sealed package during heat treatment. In an embodiment, high pressure processing comprises treating the Brassicaceae material with isostatic pressure at about 100 to about 800 MPa. In an embodiment, high pressure processing comprises treating the Brassicaceae product with isostatic pressure at about 300 to about 600 MPa. In an embodiment, high pressure processing comprises treating the Brassicaceae product with isostatic pressure at about 350 to about 550 MPa. In an embodiment, high pressure processing comprises treating the Brassicaceae product with isostatic pressure at about 300 to about 400 MPa. In an embodiment, heat treatment comprises heating the sample to a temperature of about 60° C. to about 121° C. In an embodiment, heat treatment comprises heating the sample to a temperature of about 65° C. to about 100° C. In an embodiment, heat treatment comprises heating the sample to a temperature of about 65° C. to about 80° C. In an embodiment, heat treatment comprises heating the sample to a temperature of about 65° C. to about 75° C.

A further probiotic may be added to the Brassicaceae product before or after post treatment. A person skilled in the art will appreciate that post-treatment can be performed before or after drying as described below.

### Sugar/Lyoprotectants/Cryoprotectants

In an embodiment, a sugar is added to the Brassicaceae product as described herein. In an embodiment, the added sugar is about 0.5% to about 40% of the final composition. In an embodiment, the added sugar is about 0.5% to about 30% of the final composition. In an embodiment, the added sugar is about 4% to about 25% of the final composition. In an embodiment, the added sugar is about 6% to about 20% of the final composition. In an embodiment, the added sugar is about 8% to about 18% of the final composition. In an embodiment, the added sugar is about 10% to about 15% of the final composition.

In an embodiment, the sugar may act as a lyoprotectant or cryoprotectant in a drying, cooling or freezing process. In an embodiment, the sugar is a simple sugar. In an embodiment, the sugar is selected from a monosaccharide, disaccharide or polysaccharide.

In an embodiment, a lyoprotectant/cryoptotectant is added to the Brassicaceae product as described herein. In an embodiment, the lyoprotectant/cryoprotectant is a monosaccharide, disaccharide or polysaccharide, polyalcohol or a derivative thereof. In an embodiment, the lyoprotectant/cryoprotectant is selected from one or more of: trehalose, sucrose, glycerol, maltodextrin and mannitol.

In an embodiment, the added lyoprotectant/cryoptotectant is about 0.5% to about 40% of the final composition. In an embodiment, the added lyoprotectant/cryoptotectant is about 0.5% to about 30% of the final composition. In an embodiment, the added lyoprotectant/cryoptotectant is about 4% to about 25% of the final composition. In an embodiment, the added lyoprotectant/cryoptotectant is about 6% to about 20% of the final composition. In an embodiment, the added lyoprotectant/cryoptotectant is about 8% to about 18% of the final composition. In an embodiment, the added lyoprotectant/cryoptotectant is about 10% to about 15% of the final composition.

### Drying

In an embodiment, the Brassicaceae product as described herein is partially dried or dried to reduce the water content and/or water activity. In an embodiment, the method as described herein comprises drying the Brassicaceae product to reduce the water content to about 1 to about 14%. In an embodiment, the method as described herein comprises drying the Brassicaceae product to reduce the water content to about 1 to about 13%. In an embodiment, the method comprises drying the Brassicaceae product to reduce the water content to about 1 to about 12%. In an embodiment, the method comprises drying the Brassicaceae product to reduce the water content to about 1 to about 10%. In an embodiment, the method comprises drying the Brassicaceae product to reduce the water content to about 2 to about 8%. In an embodiment, the method comprises drying the Brassicaceae product to reduce the water content to about 2 to about 6%. In an embodiment, the method comprises drying the Brassicaceae product to reduce the water content to about 2 to about 4%. In an embodiment, the method comprises drying the Brassicaceae product to reduce the water content to about 2 to about 3%.

In an embodiment, the method as described herein comprises drying the Brassicaceae product to reduce the water activity to a low water activity to about 0.1 to about 0.7. In an embodiment, the method comprises drying the Brassicaceae product to reduce the water activity to a low water activity to about 0.2 to about 0.6. In an embodiment, the method comprises drying the Brassicaceae product to reduce the water activity to a low water activity to about 0.2 to about 0.5. In an embodiment, the method comprises drying the Brassicaceae product to reduce the water activity to a low water activity to about 0.3 to about 0.4. In an embodiment, the method comprises drying the Brassicaceae product to reduce the water activity to a low water activity of about 0.4.

In an embodiment, the method as described herein comprises drying the Brassicaceae product to form a powder. Drying may include for example spray drying, freeze-drying (lyophilisation or cryodesiccation), tray drying, drum drying, roller drying, fluid bed drying, impingement drying, refractance windows drying, thin-film belt drying, vacuum microwave drying, ultrasonic-assisted drying, extrusion porosification technology or any other method known to a person skilled in the art.

In an embodiment, the Brassicaceae product is dried to produce a mean dry particle size of about 10 μM to about 4000 μM. In an embodiment, the Brassicaceae product is dried to produce a mean dry particle size of about 10 μM to about 3000 μM. In an embodiment, the Brassicaceae product is dried to produce a mean dry particle size of about 20 μM to about 2000 μM. In an embodiment, the Brassicaceae product is dried to produce a mean dry particle size of about 10 μM to about 1000 μM. In an embodiment, the Brassicaceae product is dried to produce a mean dry particle size of about 10 μM to about 500 μM.

In an embodiment, the Brassicaceae product is dried by spray drying (e.g. a Drytec laboratory spray dryer) to form a powder. For example, the Brassicaceae product is dried using a Drytec laboratory spray dryer with a rotary atomiser, ultrasonic nozzle or twin fluid nozzle at 2.0-4.0 bar atomising pressure by heating the feed to 60° C. prior to atomisation and the inlet and outlet air temperatures were 180° C. and 80° C., respectively. In an embodiment, the spray dryer has a granulation function. In an embodiment, the spray dryer is mounted with a granulation dryer.

In an embodiment, spray drying produces individual particles or agglomerates of particles.

In an embodiment, spray drying produces a mean dry particle size of about 10 μM to about 3000 μM. In an embodiment, spray drying produces a mean dry particle size of about 20 μM to about 2000 μM. In an embodiment, spray drying produces a mean dry particle size of about 10 μM to about 1000 μM. In an embodiment, spray drying produces a mean dry particle size of about 10 μM to about 500 μM.

In an embodiment, the Brassicaceae product is dried by freeze-drying to form a powder. In an embodiment, a cryoprotectant is added to the Brassicaceae product before freeze drying. In an embodiment, the cryoprotectant is a monosaccharide, disaccharide or polysaccharide, polyalcohol or a derivative thereof. In an embodiment, the cryoprotectant is selected from one or more of: trehalose, sucrose, glycerol, maltodextrin and mannitol.

In an embodiment, the Brassicaceae product is dried by drum drying to form a powder.

In an embodiment, the powder comprises about 5% to about 50% oil w/w. In an embodiment, the powder about 10% to about 50% oil w/w. In an embodiment, the powder comprises about 20% to about 50% oil w/w. In an embodiment, the powder comprises about 20% to about 50% oil w/w. In an embodiment, the powder comprises about 20% to about 40% oil w/v. In an embodiment, the powder comprises about 20% to about 30% oil w/w.

In an embodiment, the powder comprises particles of about 20 μm to about 1200 μm. In an embodiment, the powder comprises particles of about 100 μm to about 900 μm. In an embodiment, the powder comprises particles of about 400 μm to about 700 μm. In an embodiment, the powder comprises particles of about 500 μm to about 600 μm. In an embodiment, the powder comprises particles of about 1000 μm. In an embodiment, the powder is milled to further reduce the particle size. In an embodiment, milling may reduce the particle size to less than about 10 μm, or less than about 8 μm, or less than about 6 μm, or less than about 4 μm, or less than about 2 μm.

### Isolated Strains and Starter Cultures

In an embodiment, the present invention provides isolated strains of lactic acid bacteria suitable for use in the methods, compositions and delivery vehicles as described herein.

In an embodiment, the present invention provides an isolated strain of lactic acid bacteria selected from:

i) BF1 deposited under V17/021729 on 25 Sep. 2017 at the National Measurement Institute Australia;

ii) BF2 deposited under V17/021730 on 25 Sep. 2017 at the National Measurement Institute Australia;

iii) B1 deposited under V17/021731 on 25 Sep. 2017 at the National Measurement Institute Australia;

iv) B2 deposited under V17/021732 on 25 Sep. 2017 at the National Measurement Institute Australia;

v) B3 deposited under V17/021733 on 25 Sep. 2017 at the National Measurement Institute Australia;

vi) B4 deposited under V17/021734 on 25 Sep. 2017 at the National Measurement Institute Australia; and

vii) B5 deposited under V17/021735 on 25 Sep. 2017 at the National Measurement Institute Australia.

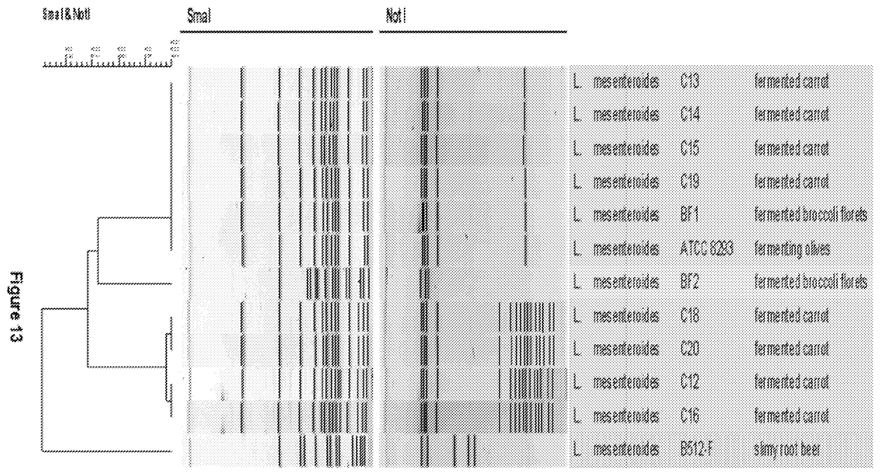

In an embodiment, the present invention provides an isolated strain of Leuconostoc mesenteroides comprising genomic DNA which when cleaved with SmaI and/or NotI produces a SmaI and/or NotI fingerprint identical to BF1 or BF2. The SmaI and NotI fingerprints for BF1 and BF2 are shown in FIG. 13.

In an embodiment, the present invention provides an isolated strain of Lactobacillus plantarum comprising genomic DNA which when cleaved with SmaI and/or NotI produces a SmaI and/or NotI fingerprint identical to B1, B2, B3, B4 or B5.

In an embodiment, the present invention provides an isolated strain of Leuconostoc mesenteroides comprising one or more or all of the polymorphisms listed in Table 18 or 19 that differs from ATCC8293. In an embodiment, the isolated strain of Leuconostoc mesenteroides comprises 5 or more of the polymorphisms listed in Table 18 or 19 that differs from ATCC8293. In an embodiment, the isolated strain of Leuconostoc mesenteroides comprises 10 or more of the polymorphisms listed in Table 18 or 19 that differs from ATCC8293. In an embodiment, the isolated strain of Leuconostoc mesenteroides comprises 15 or more of the polymorphisms listed in Table 18 or 19 that differs from ATCC8293. In an embodiment, the isolated strain of Leuconostoc mesenteroides comprises 19 or more of the polymorphisms listed in Table 18 or 19 that differs from ATCC8293. In an embodiment, the isolated strain of Leuconostoc mesenteroides comprises 20 or more of the polymorphisms listed in Table 19 that differs from ATCC8293. In an embodiment, the isolated strain of Leuconostoc mesenteroides comprises 30 or more of the polymorphisms listed in Table 19 that differs from ATCC8293. In an embodiment, the isolated strain of Leuconostoc mesenteroides comprises 50 or more of the polymorphisms listed in Table 19 that differs from ATCC8293. In an embodiment, the isolated strain of Leuconostoc mesenteroides comprises 80 or more of the polymorphisms listed in Table 19 that differs from ATCC8293. In an embodiment, the isolated strain of Leuconostoc mesenteroides comprises 100 or more of the polymorphisms listed in Table 19 that differs from ATCC8293. In an embodiment, the isolated strain of Leuconostoc mesenteroides comprises 150 or more of the polymorphisms listed in Table 19 that differs from ATCC8293. In an embodiment, the isolated strain of Leuconostoc mesenteroides comprises 200 or more of the polymorphisms listed in Table 19 that differs from ATCC8293. In an embodiment, the isolated strain of Leuconostoc mesenteroides comprises 300 or more of the polymorphisms listed in Table 19 that differs from ATCC8293. In an embodiment, the isolated strain of Leuconostoc mesenteroides comprises 400 or more of the polymorphisms listed in Table 19 that differs from ATCC8293.

In an embodiment, the present invention provides an isolated strain of Lactobacillus plantarum comprising one or more or all the polymorphisms listed in Table 13, Table 14, Table 15, Table 16 or Table 17 that differs from ATCC8014. In an embodiment, the present invention provides an isolated strain of Lactobacillus plantarum comprising 5 or more of the polymorphisms listed in Table 13, Table 14, Table 15, Table 16 or Table 17 that differs from ATCC8014. In an embodiment, the present invention provides an isolated strain of Lactobacillus plantarum comprising 10 or more of the polymorphisms listed in Table 13, Table 14, Table 15, Table 16 or Table 17 that differs from ATCC8014. In an embodiment, the present invention provides an isolated strain of Lactobacillus plantarum comprising 15 or more of the polymorphisms listed in Table 13, Table 14, Table 15, Table 16 or Table 17 that differs from ATCC8014. In an embodiment, the present invention provides an isolated strain of Lactobacillus plantarum comprising 20 or more of the polymorphisms listed in Table 13, Table 14, Table 15, Table 16 or Table 17 that differs from ATCC8014. In an embodiment, the present invention provides an isolated strain of Lactobacillus plantarum comprising 25 or more of the polymorphisms listed in Table 13, Table 14, Table 15, Table 16 or Table 17 that differs from ATCC8014. In an embodiment, the present invention provides an isolated strain of Lactobacillus plantarum comprising 30 or more of the polymorphisms listed in Table 13, Table 14, Table 15, Table 16 or Table 17 that differs from ATCC8014. In an embodiment, the present invention provides an isolated strain of Lactobacillus plantarum comprising 35 or more of the polymorphisms listed in Table 13, Table 14, Table 15, Table 16 or Table 17 that differs from ATCC8014. In an embodiment, the present invention provides an isolated strain of Lactobacillus plantarum comprising 40 or more of the polymorphisms listed in Table 13, Table 14, Table 15, Table 16 or Table 17 that differs from ATCC8014.

In an embodiment, the present invention provides a starter culture for producing a Brassicaceae product, a prebiotic, a combined prebiotic and probiotic, or a synbiotic comprising one or more of the isolated strains as described herein. As used herein a “starter culture” is a culture of live microorganisms for fermentation. In an embodiment, the present invention provides a starter culture for producing a Brassicaceae product, a prebiotic, a combined prebiotic and probiotic, or a synbiotic comprising lactic acid bacteria selected from one or more or all of:

i) BF1 deposited under V17/021729 on 25 Sep. 2017 at the National Measurement Institute Australia;

ii) BF2 deposited under V17/021730 on 25 Sep. 2017 at the National Measurement Institute Australia;

iii) B1 deposited under V17/021731 on 25 Sep. 2017 at the National Measurement Institute Australia;

iv) B2 deposited under V17/021732 on 25 Sep. 2017 at the National Measurement Institute Australia;

v) B3 deposited under V17/021733 on 25 Sep. 2017 at the National Measurement Institute Australia;

vi) B4 deposited under V17/021734 on 25 Sep. 2017 at the National Measurement Institute Australia; and

vii) B5 deposited under V17/021735 on 25 Sep. 2017 at the National Measurement Institute Australia.

In an embodiment, the Brassicaceae material is inoculated with at least about 10⁵ CFU/g of a starter culture as described herein. In an embodiment, the Brassicaceae material is inoculated with at least 10⁶ about CFU/g of a starter culture as described herein. In an embodiment, the Brassicaceae material is inoculated with at least about 10⁷ CFU/g of a starter culture as described herein. In an embodiment, the Brassicaceae material is inoculated with at least about 10⁸ CFU/g of a starter culture as described herein. In an embodiment, the Brassicaceae material is inoculated with at least about 10¹⁰ CFU/g of a starter culture as described herein. In an embodiment, the Brassicaceae material is inoculated with about 10⁵ CFU/g to about 10¹⁰ CFU/g of a starter culture as described herein.

### Glucosinolates

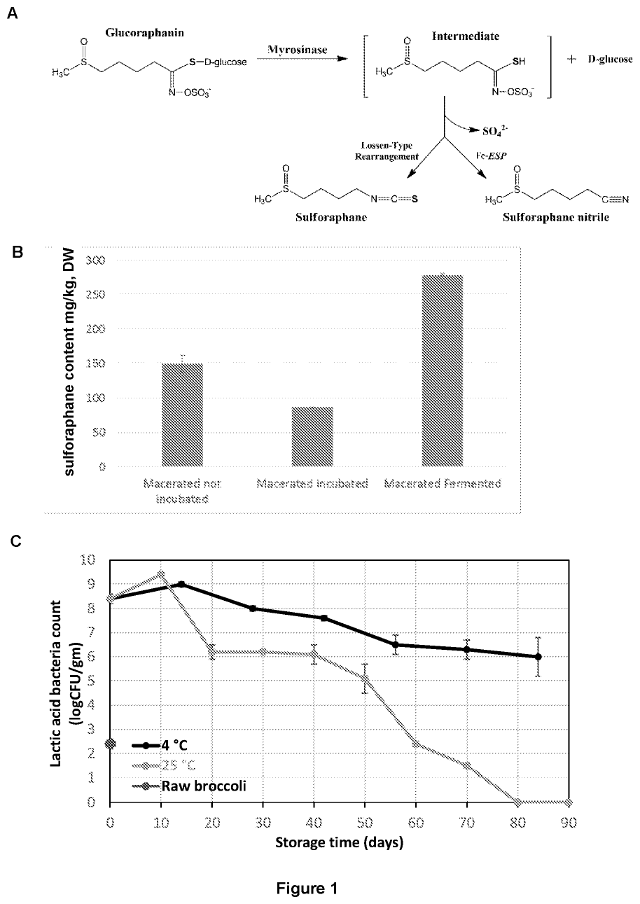

As used herein “glucosinolate” refers to a secondary metabolite found at least in the Brassicaceae family that share a chemical structure consisting of a 13-D-glucopyranose residue linked via a sulfur atom to a (Z)-N-hydroximinosulfate ester, plus a variable R group derived from an amino acid as described in Halkier et al. (2006). Examples of glucosinolates are provided in Halkier et al. (2006) and Agerbirk et al. (2012). The hydrolysis of glucosinolate can produce isothiocyanates, nitriles, epithionitrile, thiocyanate and oxazolidine-2-thione (FIG. 1A). Many glucosinolates play a role in plant defence mechanisms against pests and disease.

Glucosinolates are stored in Brassicaceae in storage sites. As used herein, a “storage site” is a site within the Brassicaceae where glucosinolates are present and myrosinase is not present.

As used herein “myrosinase” also referred to as “thioglucosidase”, “sinigrase”, or “sinigrinase” refers to a family of enzymes (EC 3.2.1.147) involved in plant defence mechanisms that can cleave thio-linked glucose. Myrosinases catalyze the hydrolysis of glucosinolates resulting in the production of isothiocyanates. Myrosinase is stored sometimes as myrosin grains in the vacuoles of particular idioblasts called myrosin cells, but have also been reported in protein bodies or vacuoles, and as cytosolic enzymes that tend to bind to membranes. Thus, in an embodiment, myrosinase is stored in a myrosin cell in Brassicaceae.

In an embodiment, pre-treating as described herein improves the access of myrosinase to a glucosinolate. As used herein “improves the access” or “access is improved” refers to increasing the availability of glucosinolate to the myrosinase enzyme allowing for the production of an isothiocyanate. In an embodiment, access is improved by the release of a glucosinolate from a glucosinolate storage site. In an embodiment, the glucosinolate storage site is mechanically ruptured (i.e. by maceration) or enzymatically degraded. In an embodiment, glucosinolate is released from a glucosinolate storage site by the activity of one or more polysaccharide degrading enzymes e.g. a cellulase, hemicellulase, pectinase and/or glycosidase. In an embodiment, access is improved by allowing the entry of myrosinase into a glucosinolate storage site. In an embodiment, access is improved by the release of myrosinase from myrosin cells. In an embodiment, about 10% to about 90% of a glucosinolate is released from a glucosinolate storage site. In an embodiment, about 10% to about 80% of a glucosinolate is released from a glucosinolate storage site. In an embodiment, about 30% to about 70% of a glucosinolate is released from a glucosinolate storage site. In an embodiment, about 40% to about 60% of a glucosinolate is released from a glucosinolate storage site. In an embodiment, about 45% to about 55% of a glucosinolate is released from a glucosinolate storage site.

In an embodiment, the Brassicaceae material comprises one or more glucosinolate/s selected from an aliphatic, indole or aromatic glucosinolate.

In an embodiment, the aliphatic glucosinolate is selected from one or more of glucoraphanin (4-Methylsulphinylbutyl or glucorafanin), sinigrin (2-Propenyl), gluconapin (3-Butenyl), glucobrassicanapin (4-Pentenyl), progoitrin (2(R)-2-Hydroxy-3-butenyl, epiprogoitrin (2(S)-2-Hydroxy-3-butenyl), gluconapoleiferin (2-Hydroxy-4-pentenyl), glucoibervirin (3-Methylthiopropyl, glucoerucin (4-Methylthiobutyl), dehydroerucin (4-Methylthio-3-butenyl, glucoiberin (3-Methylsulphinylpropyl), glucoraphenin (4-Me thylsulphinyl-3-butenyl), glucoalyssin (5-Methylsulphinylpentenyl), and glucoerysolin (3-Methylsulphonylbutyl, 4-Mercaptobutyl).

In an embodiment, the indole glucosinolate is selected from one or more of glucobrassicin (3-Indolylmethyl), 4-hydroxyglucobrassicin (4-Hydroxy-3-indolylmethyl), 4-methoxyglucobrassicin (4-Methoxy-3-indolylmethyl), and neoglucobrassicin (1-Methoxy-3-indolylmethyl).

In an embodiment, the indole glucosinolate is selected from one or more of Glucotropaeolin (Benzyl) and Gluconasturtiin (2-Phenylethyl).

In an embodiment, the Brassicaceae material comprises one or more glucosinolate/s selected from benzylglucosinolate, allylglucosinolate and 4-methylsulfinylbutyl. In an embodiment, the glucosinolate is glucoraphanin (4-Methylsulphinylbutyl). In an embodiment, the glucosinolate is glucobrassicin (3-Indolylmethyl).

In an embodiment, pre-treating as described herein increases the extractable glucosinolate content compared to the extractable glucosinolate content of the Brassicaceae material before pre-treatment.

As used herein “extractable glucosinolate content” refers to the level of glucosinolate accessible in the Brassicaceae material for conversion to isothiocyanate. Excluding conversion into nitriles and other compounds the expected maximum yield of isothiocyanate from 1 mole of glucosinolate is 1 mole of isothiocyanate (1 mole of glucosinolate can maximally be converted to 1 mole of isothiocyanate, 1 mole of glucose and 1 mole of sulphate ion). Thus, in one example, the extractable glucoraphanin content of a commercial broccoli cultivar is 3400 μmol glucoraphanin/kg dw and the expected maximum yield of sulforaphane from the commercial broccoli cultivar is 3400 μmol sulforaphane/kg dw.

### Isothiocyanates

As used herein “isothiocyanate” refers to sulphur containing phytochemicals with the general structure R—N═C═S which are a product of myrosinase activity upon a glucosinolate and bioactive derivatives thereof. In an embodiment, the isothiocyanate is sulforaphane (1-isothiocyanato-4-methylsulfinylbutane). In an embodiment, the isothiocyanate is allyl isothiocyanate (3-isothiocyanato-1-propene). In an embodiment, the isothiocyanate is benzyl isothiocyanate. In an embodiment, the isothiocyanate is phenethyl isothiocyanate. In an embodiment, the isothiocyanate is 3-Butenyl isothiocyanate. In an embodiment, the isothiocyanate is 5-vinyl-1,3-oxazolidine-2-thione. In an embodiment, the isothiocyanate is 3-(methylthio)propyl isothiocyanate. In an embodiment, the isothiocyanate is 3-(methylsulfinyl)-propyl isothiocyanate. In an embodiment, the isothiocyanate is 4-(methylthio)-butyl isothiocyanate. In an embodiment, the isothiocyanate is 1-methoxyindol-3-carbinol isothiocyanate. In an embodiment, the isothiocyanate is 2-phenylethyl isothiocyanate. In an embodiment, the isothiocyanate is iberin.

In an embodiment, the Brassicaceae product, further comprises one or more isothiocyanate bioactive derivative/s or oligomers thereof. In an embodiment, the isothiocyanate bioactive derivative is a derivative of any of the isothiocyanates as described herein. In an embodiment, the isothiocyanate bioactive derivative is a derivative of sulforaphane. In an embodiment, the isothiocyanate bioactive derivative is a derivative of iberin. In an embodiment, the isothiocyanate bioactive derivative is a derivative of allyl isothiocyanate. In an embodiment, the isothiocyanate bioactive derivative is indole-3-caribinol. In an embodiment, the isothiocyanate bioactive derivative is methoxy-indole-3-carbinol. In an embodiment, the isothiocyanate bioactive derivative is ascorbigen. In an embodiment, the isothiocyanate bioactive derivative is neoascorbigen.

### Fermented Brassicaceae Product

In an embodiment, a Brassicaceae product fermented with lactic acid bacteria (also referred to as a fermented Brassicaceae product) as described herein comprises a higher level of a prebiotic and/or a prebiotic precursor compared to the Brassicaceae material. In an embodiment, the Brassicaceae product comprises a prebiotic as described herein. In an embodiment, the Brassicaceae product comprises a prebiotic and a probiotic as described herein. In an embodiment, the Brassicaceae product comprises a prebiotic and a probiotic which are synbiotic as described herein.

In an embodiment, fermented Brassicaceae product produced by the methods as described herein comprises a higher level of isothiocyanate compared to the Brassicaceae material. For example, macerated broccoli from a commercial broccoli cultivar has a sulforaphane concentration of ˜800 μmol/Kg dw (˜149.8 mg/Kg dw), fermented macerated broccoli has a sulforaphane concentration of ˜1600 μmol/Kg dw (˜278.8 mg/Kg dw) and pre-treated and fermented broccoli produced using the methods as described herein has a sulforaphane concentration of ˜13100 μmol/Kg dw (˜2318.7 mg/Kg dw).

In an embodiment, the Brassicaceae product comprises at least about 4 times more isothiocyanate than macerated Brassicaceae material. In an embodiment, the Brassicaceae product comprises at least about 6 times more isothiocyanate than the macerated Brassicaceae material. In an embodiment, the Brassicaceae product comprises at least about 8 times more isothiocyanate than the macerated Brassicaceae material. In an embodiment, the Brassicaceae product comprises at least about 10 times more isothiocyanate than the macerated Brassicaceae material. In an embodiment, the Brassicaceae product comprises at least about 12 times more isothiocyanate than the macerated Brassicaceae material. In an embodiment, the Brassicaceae product comprises at least about 14 times more isothiocyanate than the macerated Brassicaceae material. In an embodiment, the Brassicaceae product comprises at least about 16 times more isothiocyanate than the macerated Brassicaceae material. In an embodiment, the Brassicaceae product comprises at least about 17 times more isothiocyanate than the macerated Brassicaceae material. In an embodiment, the Brassicaceae product comprises about 4 times to about 17 times more isothiocyanate than the macerated Brassicaceae material. In an embodiment, the Brassicaceae product comprises about 4 times to about 16 times more isothiocyanate than the macerated Brassicaceae material. In an embodiment, the Brassicaceae product comprises about 8 times to about 16 times more isothiocyanate than the macerated Brassicaceae material. In an embodiment, the Brassicaceae product comprises about 10 times to about 16 times more isothiocyanate than the macerated Brassicaceae material. In an embodiment, the Brassicaceae product comprises about 12 times to about 16 times more isothiocyanate than the macerated Brassicaceae material. In an embodiment, the Brassicaceae product comprises about 14 times to about 16 times more isothiocyanate than the macerated Brassicaceae material. In an embodiment, the isothiocyanate is sulforaphane.

In an embodiment, the level of isothiocyanate present in the Brassicaceae product is higher than what would be expected from the extractable glucosinolate content of the Brassicaceae material. In an embodiment, the Brassicaceae product comprises at least about 1 times the expected maximum yield of isothiocyanate based on the extractable glucosinolate content. In an embodiment, the Brassicaceae product comprises at least about 2 times the expected maximum yield of isothiocyanate based on the extractable glucosinolate content. In an embodiment, the Brassicaceae product comprises at least about 3 times the expected maximum yield of isothiocyanate based on the extractable glucosinolate content. In an embodiment, the Brassicaceae product comprises at least about 3.8 times the expected maximum yield of isothiocyanate based on the extractable glucosinolate content. In an embodiment, the Brassicaceae product comprises at least about 4 times the expected maximum yield of isothiocyanate based on the extractable glucosinolate content. In an embodiment, the Brassicaceae product comprises about 1 times to about 4 times the expected maximum yield of isothiocyanate based on the extractable glucosinolate content. In an embodiment, the Brassicaceae product comprises about 1 times to about 3.8 times the expected maximum yield of isothiocyanate based on the extractable glucosinolate content. In an embodiment, the Brassicaceae product comprises about 2 times to about 3.8 times the expected maximum yield of isothiocyanate based on the extractable glucosinolate content. In an embodiment, the Brassicaceae product comprises about 2 times to about 3 times the expected maximum yield of isothiocyanate based on the extractable glucosinolate content.

In an embodiment, the level of sulforaphane present in the Brassicaceae product is higher than what would be expected from the extractable glucoraphanin content of the Brassicaceae material. In an embodiment, the Brassicaceae product comprises at least about 1 times the expected maximum yield of sulforaphane based on the extractable glucoraphanin content. In an embodiment, the Brassicaceae product comprises at least about 2 times the expected maximum yield of sulforaphane based on the extractable glucoraphanin content. In an embodiment, the Brassicaceae product comprises at least about 3 times the expected maximum yield of sulforaphane based on the extractable glucoraphanin content. In an embodiment, the Brassicaceae product comprises at least about 3.8 times the expected maximum yield of sulforaphane based on the extractable glucoraphanin content. In an embodiment, the Brassicaceae product comprises at least about 4 times the expected maximum yield of sulforaphane based on the extractable glucoraphanin content. In an embodiment, the Brassicaceae product comprises about 1 times to about 4 times the expected maximum yield of sulforaphane based on the extractable glucoraphanin content. In an embodiment, the Brassicaceae product comprises about 1 times to about 3.8 times the expected maximum yield of sulforaphane based on the extractable glucoraphanin content. In an embodiment, the Brassicaceae product comprises about 1 times to about 3 times the expected maximum yield of sulforaphane based on the extractable glucoraphanin content. In an embodiment, the Brassicaceae product comprises about 2 times to about 3 times the expected maximum yield of sulforaphane based on the extractable glucoraphanin content.

In an embodiment, the Brassicaceae product comprises about 100 mg/kg dw to about 7000 mg/kg dw of isothiocyanate. In an embodiment, the Brassicaceae product comprises about 500 mg/kg dw to about 7000 mg/kg dw of isothiocyanate. In an embodiment, the Brassicaceae product comprises about 1000 mg/kg dw to about 7000 mg/kg dw of isothiocyanate. In an embodiment, the Brassicaceae product comprises about 1600 mg/kg dw to about 4000 mg/kg dw of isothiocyanate. In an embodiment, the Brassicaceae product comprises about 1600 mg/kg dw to about 3000 mg/kg dw of isothiocyanate. In an embodiment, the Brassicaceae product comprises about 2000 mg/kg dw to about 4000 mg/kg dw of isothiocyanate. In an embodiment, the Brassicaceae product comprises about 2000 mg/kg dw of to about 7000 mg/kg dw of isothiocyanate. In an embodiment, the Brassicaceae product comprises about 3000 mg/kg dw isothiocyanate to about 7000 mg/kg of isothiocyanate. In an embodiment, the Brassicaceae product comprises about 2300 mg/kg dw of the isothiocyanate.

In an embodiment, the Brassicaceae product comprises at least about 100 mg/kg dw of the isothiocyanate. In an embodiment, the Brassicaceae product comprises at least about 200 mg/kg dw of the isothiocyanate. In an embodiment, the Brassicaceae product comprises at least about 250 mg/kg dw of the isothiocyanate. In an embodiment, the Brassicaceae product comprises at least about 300 mg/kg dw of the isothiocyanate. In an embodiment, the Brassicaceae product comprises at least about 350 mg/kg dw of the isothiocyanate. In an embodiment, the Brassicaceae product comprises at least about 400 mg/kg dw of the isothiocyanate. In an embodiment, the Brassicaceae product comprises at least about 450 mg/kg dw of the isothiocyanate. In an embodiment, the Brassicaceae product comprises at least about 500 mg/kg dw of the isothiocyanate. In an embodiment, the Brassicaceae product comprises at least about 550 mg/kg dw of the isothiocyanate. In an embodiment, the Brassicaceae product comprises at least about 600 mg/kg dw of the isothiocyanate. In an embodiment, the Brassicaceae product comprises at least about 650 mg/kg dw of the isothiocyanate. In an embodiment, the Brassicaceae product comprises at least about 700 mg/kg dw of the isothiocyanate. In an embodiment, the Brassicaceae product comprises at least about 1000 mg/kg dw of the isothiocyanate. In an embodiment, the Brassicaceae product comprises at least about 2000 mg/kg dw of the isothiocyanate. In an embodiment, the Brassicaceae product comprises at least about 3000 mg/kg dw of the isothiocyanate. In an embodiment, the Brassicaceae product comprises at least about 4000 mg/kg dw of the isothiocyanate. In an embodiment, the Brassicaceae product comprises at least about 5000 mg/kg dw of the isothiocyanate. In an embodiment, the Brassicaceae product comprises at least about 6000 mg/kg dw of the isothiocyanate. In an embodiment, the Brassicaceae product comprises at least about 7000 mg/kg dw of the isothiocyanate.

In an embodiment, the Brassicaceae product comprises at least about 100 mg/kg dw of sulforaphane. In an embodiment, the Brassicaceae product comprises at least about 150 mg/kg of sulforaphane. In an embodiment, the Brassicaceae product comprises at least about 200 mg/kg dw of sulforaphane. In an embodiment, the Brassicaceae product comprises at least about 250 mg/kg of sulforaphane. In an embodiment, the Brassicaceae product comprises at least about 300 mg/kg dw of sulforaphane. In an embodiment, the Brassicaceae product comprises at least about 350 mg/kg dw of sulforaphane. In an embodiment, the Brassicaceae product comprises at least about 400 mg/kg dw of sulforaphane. In an embodiment, the Brassicaceae product comprises at least about 450 mg/kg dw of sulforaphane. In an embodiment, the Brassicaceae product comprises at least about 500 mg/kg dw of sulforaphane. In an embodiment, the Brassicaceae product comprises at least about 550 mg/kg dw of sulforaphane. In an embodiment, the Brassicaceae product comprises at least about 600 mg/kg dw of sulforaphane. In an embodiment, the Brassicaceae product comprises at least about 650 mg/kg dw of sulforaphane. In an embodiment, the Brassicaceae product comprises at least about 700 mg/kg dw of sulforaphane. In an embodiment, the Brassicaceae product comprises at least about 1000 mg/kg of sulforaphane dw. In an embodiment, the Brassicaceae product comprises at least about 2000 mg/kg dw of sulforaphane. In an embodiment, the Brassicaceae product comprises at least about 3000 mg/kg dw of sulforaphane. In an embodiment, the Brassicaceae product comprises at least about 4000 mg/kg dw of sulforaphane. In an embodiment, the Brassicaceae product comprises at least about 5000 mg/kg dw of sulforaphane. In an embodiment, the Brassicaceae product comprises at least about 6000 mg/kg dw of sulforaphane. In an embodiment, the Brassicaceae product comprises at least about 7000 mg/kg dw of sulforaphane.

In an embodiment, the Brassicaceae product comprises at least about 5% more total fibre than the Brassicaceae material. In an embodiment, the Brassicaceae product comprises at least about 10% more total fibre than the Brassicaceae material. In an embodiment, the Brassicaceae product comprises at least about 15% more total fibre than the Brassicaceae material. In an embodiment, the Brassicaceae product comprises at least about 20% more total fibre than the Brassicaceae material. In an embodiment, the Brassicaceae product comprises at least about 4% more protein than the Brassicaceae material. In an embodiment, the Brassicaceae product comprises at least about 6% more protein than the Brassicaceae material. In an embodiment, the Brassicaceae product comprises at least about 8% more protein than the Brassicaceae material. In an embodiment, the Brassicaceae product comprises at least about 10% more protein than the Brassicaceae material.

In an embodiment, the Brassicaceae product comprises at least about 10% less carbohydrate than the Brassicaceae material. In an embodiment, the Brassicaceae product comprises at least about 20% less carbohydrate than the Brassicaceae material. In an embodiment, the Brassicaceae product comprises at least about 30% less carbohydrate than the Brassicaceae material. In an embodiment, the Brassicaceae product comprises at least about 40% less carbohydrate than the Brassicaceae material. In an embodiment, the Brassicaceae product comprises at least about 45% less carbohydrate than the Brassicaceae material. In an embodiment, the Brassicaceae product comprises at least about 48% less carbohydrate than the Brassicaceae material. In an embodiment, the Brassicaceae product comprises about 10% to about 48% less carbohydrate than the Brassicaceae material.

In an embodiment, the Brassicaceae product comprises an increased level of polyphenolic glycosides compared to the Brassicaceae material. In an embodiment, the polyphenolic glycosides are anthocyanin glycosides. In an embodiment, the polyphenolic glycosides are phenolic acid glycosides. In an embodiment, the polyphenolic glycosides are phenolic acids.

In an embodiment, the Brassicaceae product comprises an increased level of glucosinolates compared to the Brassicaceae material. In an embodiment, the glucosinolate is glucoraphanin. In an embodiment, glucoraphanin is increased at least about 25 fold. In an embodiment, the glucosinolate is glucobrassicin. In an embodiment, the glucobrassicin is increased by 26 times. In an embodiment, the Brassicaceae product comprises indole-3-carbinol. In an embodiment, indol-3carbinol is increased at least about 2 fold in the Brassicaceae product compared to the macerated Brassicaceae material. In an embodiment, indol-3-carbinol is increased at least about 3 fold in the Brassicaceae product compared to the macerated Brassicaceae material. In an embodiment, the Brassicaceae product comprises ascorbigen. In an embodiment, ascorbigen is increased at least about 2 fold in the Brassicaceae product compared to the macerated Brassicaceae material. In an embodiment, ascorbigen is increased at least about 3 fold in the Brassicaceae product compared to the macerated Brassicaceae material.

In an embodiment, the Brassicaceae product comprises an increased level of one or more of ferullic acid, syringic acid, phenyllactic acid, chlorogenic acid rutin, sinapic acid, methyl syringate, hesperetin, quercetin and kaempferol compared to the Brassicaceae material. In an embodiment, the Brassicaceae product comprises an increased level of chlorogenic acid compared to the Brassicaceae material. In an embodiment, chlorogenic acid is increased about 6.6 fold. In an embodiment, the Brassicaceae product comprises an increased level of sinapic acid compared to the Brassicaceae material. In an embodiment, sinapic acid is increased about 23.8 fold. In an embodiment, the Brassicaceae product comprises an increased level of kaempferol compared to the Brassicaceae material. In an embodiment, kaempferol is increased about 10.5 fold.

In an embodiment, the Brassicaceae product comprises an decreased level of one or more of protocatechuic acid, gallic acid, 4,hydroxybenzoic acid, vanillic acid, 2,3dihydroxybenzoic acid, p-cuomaric acid, cinnamic acid, catechin, rosmarinic acid, caffeic acid compared to the Brassicaceae material.

In an embodiment, about 40% of a glucosinolate present in the Brassicaceae material is converted to an isothiocyanate in the Brassicaceae product. In an embodiment, about 50% of a glucosinolate present in the Brassicaceae material is converted to an isothiocyanate in the Brassicaceae product. In an embodiment, about 60% of a glucosinolate present in the Brassicaceae material is converted to an isothiocyanate in the Brassicaceae product. In an embodiment, about 70% of a glucosinolate present in the Brassicaceae material is converted to an isothiocyanate in the Brassicaceae product. In an embodiment, about 80% of a glucosinolate present in the Brassicaceae material is converted to an isothiocyanate in the Brassicaceae product. In an embodiment, about 90% of a glucosinolate present in the Brassicaceae material is converted to an isothiocyanate in the Brassicaceae product. In an embodiment, about 95% of a glucosinolate present in the Brassicaceae material is converted to an isothiocyanate in the Brassicaceae product. In an embodiment, about 97% of a glucosinolate present in the Brassicaceae material is converted to an isothiocyanate in the Brassicaceae product. In an embodiment, about 98% of a glucosinolate present in the Brassicaceae material is converted to an isothiocyanate in the Brassicaceae product. In an embodiment, about 99% of a glucosinolate present in the Brassicaceae material is converted to an isothiocyanate in the Brassicaceae product. In an embodiment, about 100% of a glucosinolate present in the Brassicaceae material is converted to an isothiocyanate in the Brassicaceae product. In an embodiment, about 40% to about 100% of a glucosinolate present in the Brassicaceae material is converted to an isothiocyanate in the Brassicaceae product. In an embodiment, about 40% to about 80% of a glucosinolate present in the Brassicaceae material is converted to an isothiocyanate in the isothiocyanate containing Brassicaceae product.

In an embodiment, the isothiocyanate in the Brassicaceae product is stable for at least a week, or for at least two weeks, or for at least 3 weeks, or for at least 4 weeks, or for at least 6 weeks, or for at least 8 weeks, or for at least 10 weeks, or for at least 12 weeks, or for at least 14 weeks when stored at about 4° C. to about 25° C. In an embodiment, the isothiocyanate in the Brassicaceae product is stable for at least 4 weeks when stored at about 4° C. to about 25° C. In an embodiment, the isothiocyanate in the Brassicaceae product is stable for at least 8 weeks when stored at about 4° C. to about 25° C. In an embodiment, the isothiocyanate in the Brassicaceae product is stable for at least 12 weeks when stored at about 4° C. to about 25° C.

As used herein “stable” refers to no decrease or only a minor decrease in isothiocyanate concentration when stored at 4° C. for six weeks. In an embodiment, a minor decrease refers to a decrease in isothiocyanate concentration of about 1% to about 30%. In an embodiment, a minor decrease refers to a decrease in isothiocyanate concentration of about 5% or less. In an embodiment, a minor decrease refers to a decrease in isothiocyanate concentration of about 10% or less. In an embodiment, a minor decrease refers to a decrease in isothiocyanate concentration of about 15% or less. In an embodiment, a minor decrease refers to a decrease in isothiocyanate concentration of about 20% or less. In an embodiment, a minor decrease refers to a decrease in isothiocyanate concentration of about 30% or less. Isothiocyanate analysis can be performed by any method know to a person skilled in the art and for example as shown in Example 1 for sulforaphane.

In an embodiment, the isothiocyanate is sulforaphane.

In an embodiment, the Brassicaceae product is resistant to yeast, mould and/or coliform growth for at least a week, or for at least two weeks, or for at least 3 weeks, or for at least 4 weeks, or for at least 6 weeks, or for at least 8 weeks, or for at least 10 weeks, or for at least 12 weeks, or for at least 14 weeks when stored at about 4° C. to about 25° C.

In an embodiment, the Brassicaceae product is resistant to yeast, mould and/or coliform growth for at least 4 weeks when stored at about 4° C. to about 25° C. In an embodiment, the Brassicaceae product is resistant to yeast, mould and/or coliform growth for at least 8 weeks when stored at about 4° C. to about 25° C. In an embodiment, the Brassicaceae product is resistant to yeast, mould and/or coliform growth for at least 12 weeks when stored at about 4° C. to about 25° C.

As used herein “resistant” to yeast, mould and/or coliform growth means that <1 Log CFU/g of yeast, mould and/or coliform is detectable in the sample after the above listed time periods using the methods described in Example 1. In an embodiment, the Brassicaceae product comprises about 20 g/100 gdw to about 32 g/100 gdw total fibre. In an embodiment, the Brassicaceae product comprises about 20 g/100 gdw total fibre. In an embodiment, the Brassicaceae product comprises about 25 g/100 gdw total fibre. In an embodiment, the Brassicaceae product comprises about 28 g/100 gdw total fibre. In an embodiment, the Brassicaceae product comprises about 29 g/100 gdw total fibre. In an embodiment, the Brassicaceae product comprises about 30 g/100 gdw total fibre. In an embodiment, the Brassicaceae product comprises about 32 g/100 gdw total fibre.

In an embodiment, the Brassicaceae product comprises an ORAC antioxidant capacity of about 14000 μmol TE/100 gdw to about 19000 μmol TE/100 gdw. In an embodiment, the Brassicaceae product comprises an ORAC antioxidant capacity of about 14000 μmol TE/100 gdw. In an embodiment, the Brassicaceae product comprises an ORAC antioxidant capacity of about 15000 μmol TE/100 gdw. In an embodiment, the Brassicaceae product comprises an ORAC antioxidant capacity of about 16000 μmol TE/100 gdw. In an embodiment, the Brassicaceae product comprises an ORAC antioxidant capacity of about 17000 μmol TE/100 gdw. In an embodiment, the Brassicaceae product comprises an ORAC antioxidant capacity of about 18000 mol TE/100 gdw. In an embodiment, the Brassicaceae product comprises an ORAC antioxidant capacity of about 18695 μmol TE/100 gdw. In an embodiment, the Brassicaceae product comprises an ORAC antioxidant capacity of about 19000 mol TE/100 gdw.

In an embodiment, the Brassicaceae product comprises a total polyphenol content of about 1750 mg GAE/100 gdw to about 2600 mg GAE/100 gdw. In an embodiment, the Brassicaceae product comprises a total polyphenol content of about 1750 mg GAE/100 gdw. In an embodiment, the Brassicaceae product comprises a total polyphenol content of about 2000 mg GAE/100 gdw. In an embodiment, the Brassicaceae product comprises a total polyphenol content of about 2100 mg GAE/100 gdw. In an embodiment, the Brassicaceae product comprises a total polyphenol content of about 2200 mg GAE/100 gdw. In an embodiment, the Brassicaceae product comprises a total polyphenol content of about 2300 mg GAE/100 gdw. In an embodiment, the Brassicaceae product comprises a total polyphenol content of about 2360 mg GAE/100 gdw.

In an embodiment, the Brassicaceae product comprises a total titratable acidity of about 0.9% to about 1.1% lactic acid equivalent. In an embodiment, the Brassicaceae product comprises a total titratable acidity of about 1.1% lactic acid equivalent.

In an embodiment, the Brassicaceae product comprises a total protein content of about 23 g/100 gdw to about 39 g/100 gdw. In an embodiment, the Brassicaceae product comprises a total protein content of about 23 g/100 gdw to about 30 g/100 gdw. In an embodiment, the Brassicaceae product comprises a total protein content of about 25 g/100 gdw. In an embodiment, the Brassicaceae product comprises a total protein content of about 27 g/100 gdw. In an embodiment, the Brassicaceae product comprises a total protein content of about 28 g/100 gdw. In an embodiment, the Brassicaceae product comprises a total protein content of about 29 g/100 gdw. In an embodiment, the Brassicaceae product comprises a total protein content of about 30 g/100 gdw. In an embodiment, the Brassicaceae product comprises a total protein content of about 32 g/100 gdw.

In an embodiment, the Brassicaceae product comprises at least about 100 mg/kg dw of an isothiocyanate and one or more or all of the following.

i) total fibre at about 29 to about 36 g/100 gdw;

ii) an ORAC antioxidant capacity of about 15000 to about 18695 μmol TE/100 gdw;

iii) a total polyphenol content of about 2310 to about 2600 mg GAE/100 gdw;

iv) a total titratable acidity of about 0.9 to about 1.1% lactic acid equivalent;

v) a total protein content of about 27 to about 39 g/100 gdw; and

vi) Leuconostoc mesenteroides and/or Lactobacillus plantarum.

In an embodiment, the Brassicaceae product is produced from broccoli.

In an embodiment, Brassicaceae product increases the production of one or more SCFA in the gastrointestinal tract in the subject. In an embodiment, the Brassicaceae product increases the production of one or more SCFA in the lower gastrointestinal tract of the subject. In an embodiment, the Brassicaceae product increases the production of one or more SCFA in the colon of the subject. In an embodiment, production of one or more SCFA is increased relative to an unfermented Brassicaceae product.

In an embodiment, the total SCFA level is increased about 30% to about 70% compared to administration of unfermented Brassicaceae. In an embodiment, the total SCFA level is increased about 38% to about 65% compared to administration of unfermented Brassicaceae. In an embodiment, the total SCFA level is increased about 40% to about 60% compared to administration of unfermented Brassicaceae. In an embodiment, the total SCFA level is increased about 40% to about 55% compared to administration of unfermented Brassicaceae.

In an embodiment, the butyrate level is increased about 30% to about 70% compared to administration of unfermented Brassicaceae. In an embodiment, the butyrate is increased about 38% to about 65% compared to administration of unfermented Brassicaceae. In an embodiment, the butyrate level is increased about 40% to about 60% compared to administration of unfermented Brassicaceae. In an embodiment, the butyrate level is increased about 40% to about 55% compared to administration of unfermented Brassicaceae.

In an embodiment, the propionate level is increased about 30% to about 70% compared to administration of unfermented Brassicaceae. In an embodiment, the propionate is increased about 38% to about 65% compared to administration of unfermented Brassicaceae. In an embodiment, the propionate level is increased about 40% to about 60% compared to administration of unfermented Brassicaceae. In an embodiment, the propionate level is increased about 40% to about 55% compared to administration of unfermented Brassicaceae.

In an embodiment, the acetate level is increased about 30% to about 70% compared to administration of unfermented Brassicaceae. In an embodiment, the acetate is increased about 38% to about 65% compared to administration of unfermented Brassicaceae. In an embodiment, the acetate level is increased about 40% to about 60% compared to administration of unfermented Brassicaceae. In an embodiment, the acetate level is increased about 40% to about 55% compared to administration of unfermented Brassicaceae.

In an embodiment, the Brassicaceae product increases the SCFA level about 5 to about 48 hours after administration. In an embodiment, the Brassicaceae product increases the SCFA level about 10 to about 24 hours after administration.

The Brassicaceae products as described herein can comprise a live probiotic as described herein. In an embodiment, fermentation of the Brassicaceae product as described herein increases the stability of the probiotic in the composition compared to a probiotic in an unfermented Brassicaceae product. In an embodiment, the fatty acid as described herein in the Brassicaceae product as described herein increases the stability of the probiotic in the Brassicaceae product compared to Brassicaceae product lacking an added fatty acid.

In an embodiment, the probiotic is a Bifidobacterim lactis. In an embodiment, the probiotic is a Bifidobacterim anamalis.

The Brassicaceae products as described herein can comprise live lactic acid bacteria which can aid the conversion of glucosinolate present in the Brassicaceae product to an isothiocyanates during digestion of a glucosinolate containing product in a subject (i.e. they act as a probiotic). In an embodiment, the lactic acid bacteria is a Leuconostoc mesenteroide. In an embodiment, the lactic acid bacteria is Lactobacillus sp. In an embodiment, the lactic acid bacteria is Lactobacillus plantarum.

In an embodiment, the Brassicaceae product comprises lactic acid bacteria at a concentration of at least about 10² CFU/g. In an embodiment, the Brassicaceae product comprises lactic acid bacteria at a concentration of at least about 10² CFU/g. In an embodiment, the Brassicaceae product comprises lactic acid bacteria at a concentration of at least about 10⁵ CFU/g. In an embodiment, the Brassicaceae product comprises lactic acid bacteria at a concentration of at least about 10⁶ CFU/g. In an embodiment, the Brassicaceae product comprises lactic acid bacteria at a concentration of at least about 10⁷ CFU/g. In an embodiment, the Brassicaceae product comprises lactic acid bacteria at a concentration of at least about 10⁸ CFU/g. In an embodiment, the Brassicaceae product comprises lactic acid bacteria at a concentration of at least about 10⁹ CFU/g.

In an embodiment, live lactic acid bacteria are present in the Brassicaceae product for at least 10 days when stored at about 4° C. to about 25° C. In an embodiment, live lactic acid bacteria are present in the Brassicaceae product at least 20 days when stored at about 4° C. to about 25° C. In an embodiment, live lactic acid bacteria are present in the Brassicaceae product at least 30 days when stored at about 4° C. to about 25° C. In an embodiment, live lactic acid bacteria are present in the Brassicaceae product at least 40 days when stored at about 4° C. to about 25° C. In an embodiment, live lactic acid bacteria are present in the Brassicaceae product at least 50 days when stored at about 4° C. to about 25° C. In an embodiment, live lactic acid bacteria are present in the Brassicaceae product at least 60 days when stored at about 4° C. to about 25° C. In an embodiment, live lactic acid bacteria are present in the Brassicaceae product at least 70 days when stored at about 4° C. to about 25° C. In an embodiment, live lactic acid bacteria are present in the Brassicaceae product at least 80 days when stored at about 4° C. to about 25° C. In an embodiment, live lactic acid bacteria are present in the Brassicaceae product at least 85 days when stored at about 4° C. to about 25° C. In an embodiment, live lactic acid bacteria are present in the Brassicaceae product at least 90 days when stored at about 4° C. to about 25° C.

In an embodiment, the lactic acid bacteria is a Lactobacillus sp. In an embodiment, the lactic acid bacteria is Lactobacillus plantarum. In an embodiment, the lactic acid bacteria is Leuconostoc mesenteroides. In an embodiment, the bacteria are present at a concentration of at least about 10⁷ CFU/g.

In an embodiment, the Brassicaceae product comprises one or more fatty acids or oils as described herein. In an embodiment, the one or more fatty acids or oils are resistant to oxygen degradation. In an embodiment, the one or more fatty acids or oils has a longer IP compared the one or more fatty acids or oils in a non-fermented Brassicaceae product. In an embodiment, the one or more fatty acids or oils has a longer IP compared the one or more fatty acids or oils in a non-fermented Brassicaceae product, when the oil is added prior to fermentation.

As used herein, the term “resistant to oxygen degradation”, “resistant to degradation by oxygen” or similar phrases, refers to reducing the susceptibility of a fatty acid or an oil to oxidation. In an embodiment, the susceptibility of the fatty acid or oil to oxidation is reduced by entrapping or encapsulating the substance to reduce exposure to oxygen. In an embodiment, this includes entrapping or encapsulating the substance with molecules with oxygen sequestration ability. Assessment of oxidative resistance may be performed by any method known to a person skilled in the art. For example, the oxidative resistance of a fatty acid or an oil may be based on the oxidation of oil with oxygen under pressure. In such a test, the consumption of oxygen, results in a pressure drop during the test which is due to the uptake of oxygen by the sample during oxidation. The oxidation rate is accelerated when carried out at elevated pressure and temperature. In an embodiment, the oxidative resistance is assessed using an Oxipres (e.g. a Mikrolab Aarhus A/S apparatus Hojbjerg, Denmark). In an embodiment, an emulsion, suspension and/or powder containing a fatty acid or an oil (e.g. polyunsaturated oils) is exposed to high temperature and high oxygen pressure. In an embodiment, the oxidative resistance is assessed at 80° C. and 5 bar initial oxygen pressure. In an embodiment, the induction period (IP, h) is determined, which is related to oxidative stability of the samples. A longer IP (h) indicates that athe sample is more resistant (more stable in the presence of oxygen) to oxidation during storage. Other methods for measuring oxidation include, for example, peroxide value, para-anisidine value and headspace analysis of volatiles (eg aldehydes such as propanal and EE-2,4-heptadienal which are secondary oxidation products from oxidation of omega-3 fatty acids) and change in % individual unsaturated fatty acids (e.g. EPA and DHA) in stored samples.

In an embodiment, the Brassicaceae product comprises one or more bacteriocin/s produced by lactic acid bacteria. In an embodiment, the bacteriocin is a Class I bacteriocin. In an embodiment, the bacteriocin is a Class II bacteriocin. In an embodiment, the bacteriocin is a Class III bacteriocin. Examples of bacteriocins produced by lactic acid bacteria can be found in Alvarez-Sieiro et al. (2016).

In an embodiment, the Brassicaceae product is a food product. In an embodiment, the Brassicaceae product is a nutraceutical. In an embodiment, the Brassicaceae product is a supplement. In an embodiment, the Brassicaceae product is a food ingredient. In an embodiment, the Brassicaceae product is a probiotic. In an embodiment, the Brassicaceae product is an animal feed. In an embodiment, the Brassicaceae product is an animal feed is in aquaculture feed. In an embodiment, the Brassicaceae product may be added to an animal feed e.g. Novacq prawn feed (CSIRO). The animal can be an aquatic animal such as fish, prawns or livestock. In an embodiment, the Brassicaceae product is a pesticide. In an embodiment, the Brassicaceae product is a cosmeceutical. In an embodiment, the Brassicaceae product is topically formulated.

In an embodiment, the Brassicaceae product is a solid, liquid, emulsion, capsule, tablet, pill. puree or a powder. In an embodiment, the Brassicaceae product is dried to a powder after fermentation. In an embodiment, the Brassicaceae product is freeze dried after fermentation. In an embodiment, the Brassicaceae product is microencapsulated as described in WO2005030229 after fermentation. In an embodiment, the Brassicaceae product is formulated as a pill.

In an embodiment, the Brassicaceae product as described herein may be microencapsulated as described in WO0174175. In an embodiment, the compositions as described herein may be microencapsulated as described in WO2014169315.

### Compositions

The present invention provides compositions comprising a fermented Brassicaceae product. In an embodiment, the composition is a food product. In an embodiment, the composition is a nutraceutical. In an embodiment, the composition is a supplement. In an embodiment, the composition is a food ingredient. In an embodiment, the composition comprises a prebiotic. In an embodiment, the composition comprises a prebiotic and a probiotic. In an embodiment, the composition is an animal feed. The animal can be an aquatic animal such as fish, prawns or livestock. In an embodiment, the composition is a pharmaceutical composition. In an embodiment, the composition is an emulsion or a suspension. In an embodiment, the pharmaceutical composition comprises one or more pharmaceutically acceptable excipients. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In an embodiment, the pharmaceutical compositions as described herein may comprise one or more further active ingredients.

### Delivery Vehicle

In an embodiment, the present invention provides a vehicle for delivering a bioactive to a subject, wherein the vehicle comprises a Brassicaceae product fermented with lactic acid bacteria, wherein the lactic acid bacteria were derived from an isolate obtained from Brassicaceae and/or the Brassicaceae product was pre-treated prior to fermentation.

In an embodiment, the Brassicaceae product comprises one or more or all of: i) a prebiotic, ii) a prebiotic and a probiotic, and iii) a prebiotic and a probiotic which are synbiotic

In an embodiment, the delivery vehicle stabilizes (reduces the degradation or loss) of a bioactive during storage and/or delivery. In an embodiment, the delivery vehicle, in instances where the bioactive is a live microorganism (e.g. a probiotic), improves the viability of the microorganism compared to the microorganism administered without the delivery vehicle.

In an embodiment, the bioactive is selected from one or more or all of:

i) a fatty acid, ii) oil, iii) a further prebiotic, and iv) a further probiotic.

Examples of fatty acid and oils are described in the “addition of fatty acid and/or oil” section of the specification.

In an embodiment, the further prebiotic is selected from one or more or all of: dietary fibre, oligosaccharides, exopolysaccharides, oligofructose, cellulose, hemicellulose resistant starch, beta-glucans pectin, inulin and dextran.

In an embodiment, the oligosaccharides are selected from one or more or all of: gluco-oligosaccharides fructo-oligosaccharides galacto-oligosaccharide, pecticoligosaccharide trans-galacto-oligosaccharides.

In an embodiment, the exopolysaccharides are homopolysaccharides and/or heteropolysaccharides.

In an embodiment, the vehicle improves the viability of a probiotic. In an embodiment, the vehicle improves the viability of the further probiotic.

As used herein, “viability” refers to a probiotics ability to survive or live successfully. An improvement in the viability of the probiotic can be an improvement during storage (e.g. an increase in hrs or days the probiotic can be stored before use) and/or improvement during delivery of the probiotic to another organism. In an embodiment, an improvement in the viability of the probiotic refers to an increase in viability of the probiotic when passing through the upper gastrointestinal tract. In an embodiment, the viability is increased by about 0.5 log to about 5 log compared to delivery without the vehicle. In an embodiment, the viability is increased by about 0.5 log to about 4 log compared to delivery without the vehicle. In an embodiment, the viability is increased by about 0.5 log to about 3 log compared to delivery without the vehicle. In an embodiment, an improvement in the viability if the probiotic refers to an increase in the delivery of the probiotic to the lower gastrointestinal tract. In an embodiment, delivery of the probiotic to the lower gastrointestinal tract is increased by 0.5 log to about 5 log compared to delivery without the vehicle. In an embodiment, delivery of the probiotic to the lower gastrointestinal tract is increased by 0.5 log to about 4 log compared to delivery without the vehicle. In an embodiment, delivery of the probiotic to the lower gastrointestinal tract is increased by 0.5 log to about 3 log compared to delivery withough the vehicle.

In an embodiment, the vehicle protects the probiotic during passage through the upper gasterintestinal tract. As used herein “protects” or “protecting” refers to reducing the suceptability of a probitic to damage and/or death caused by exposure to gastrointestinal digestive enzyme or digestive juices during passage through the upper gastrointestinal tract. In an embodiment, protects refers to reducing the suceptability of a probiotic to damage or death caused by gastric enzymes, gastric juices and/or bile during passage throught the upper gastrointestinal tract. In an embodiment, protecting the probiotic during passage through the upper gastrointestinal tract increases the amount of viabile probiotic delivered to the lower gastrointestinal tract.

In an embodiment, the probiotic of further probiotic is autochthonous to the Brassicaceae material. In an embodiment, the probiotic or further probiotic is an autochthonous probiotic is present on the Brassicaceae material before fermentation. In an embodiment, the probiotic or further probiotic is an allochthonous probiotic added to the Brassicaceae material after fermentation.

In an embodiment, the probiotic or further probiotic is selected from one or more or all of: lactic acid bacteria, Bifidobacteria, Bacteroidetes, Baciullus, Streptococcus, Escherichia, Enterococcus, Saccharomyces.

In an embodiment, the lactic acid bacteria is selected from one or more of the genera selected from: Lactobacillus, Leuconostoc, Pediococcus, Lactococcus, Streptococcus, Aerococcus, Camobacterium, Enterococcus, Oenococcus, Sporolactobacillus, Tetragenococcus, Vagococcus and Weissella. In an embodiment, the lactic acid bacteria is selected from one or more or all of: Lactobacillus plantarum, Leuconostoc mesenteroides, Lactobacillus rhamnosus, Lactobacillus pentosus, Lactobacillus brevis, Lactococus lactis, Lactobacillus acidophilus, Lactobacillus brevis, Lactobacillus casei, Lactobacillus delbrueckii, Lactobacillus fermentum, Lactobacillus gasseri, Lactobacillus johnsonii, Lactobacillus lactis, Lactobacillus paracasei, Lactobacillus reuteri, Pediococcus pentosaceus and Pedicoccus acidilacti. In an embodiment, the lactic acid bacteria is selected from one or more or all of: i) BF1 deposited under V17/021729 on 25 Sep. 2017 at the National Measurement Institute Australia; ii) BF2 deposited under V17/021730 on 25 Sep. 2017 at the National Measurement Institute Australia; iii) B1 deposited under V17/021731 on 25 Sep. 2017 at the National Measurement Institute Australia; iv) B2 deposited under V17/021732 on 25 Sep. 2017 at the National Measurement Institute Australia; v) B3 deposited under V17/021733 on 25 Sep. 2017 at the National Measurement Institute Australia; vi) B4 deposited under V17/021734 on 25 Sep. 2017 at the National Measurement Institute Australia; and vii) B5 deposited under V17/021735 on 25 Sep. 2017 at the National Measurement Institute Australia.

In embodiment, the Bifidobacteria is selected from one or more of: Bifidobacteria adolescentis, Bifidobacteria animalis, Bifidobacteria bifidum, Bifidobacteria breve, Bifidobacteria infantis, Bifidobacteria longum, and Bifidobacteria thermophilum.

In embodiment, the Baciullus is selected from one or more of: Baciullus cereus, Baciullus clausii, Baciullus coagulans, Baciullus licheniformis, Baciullus pumulis and Baciullus subtilis.

In embodiment, the Streptococcus is Streptococcus thermophiles. In embodiment, the Escherichia is beneficial strain of Escherichia coli.

In embodiment, the Enterococcus is Enterociccus faecium.

In embodiment, the Saccharomyces is Saccharomyces cerevisiae

### Administration

A variety of routes of administration are possible for the methods, compositions and delivery vehicles as described herein, including but not limited to enteral, dietary, parenteral, and topically. In an embodiment, the Brassicaceae product is administered enterally. As used herein “enterally” or “enteral” comprises passing through the gastrointestinal tract. In an embodiment, enteral administration comprises oral administration. In an embodiment, enteral administration comprises rectal administration. In an embodiment, rectal administration may be selected from one or more of: suppository, enema, via colonoscope or other medical equipment and faecal transplantation. In an embodiment, the Brassicaceae product as described herein is administered parenterally. In an embodiment, the Brassicaceae product as described herein is administered topically.

In an embodiment, the present invention provides a faecal microbiota suitable for transplantation into a subject, wherein the faecal microbiota was isolated from a subject administered a Brassicaceae product fermented with lactic acid bacteria, wherein the lactic acid bacteria were derived from an isolate obtained from Brassicaceae and/or the Brassicaceae product was pre-treated prior to fermentation.

In an embodiment, the present invention provides a digesta microbiota suitable for transplantation into a subject, wherein the faecal microbiota was isolated from a subject administered a Brassicaceae product fermented with lactic acid bacteria, wherein the lactic acid bacteria were derived from an isolate obtained from Brassicaceae and/or the Brassicaceae product was pre-treated prior to fermentation.

### EXAMPLES

### Example 1—Methods

### Chemicals and Reagents

HPLC grade methanol, sodium dihydrogen phosphate, sodium hydroxide (NaOH) and hydrochloric acid (HCl) were purchased from Merck (Damstadt, Germany). Folin-Ciocalteu's reagent, sodium carbonate (Na₂CO₃), gallic acid, fluorescein sodium salt and dibasic-potassium phosphate were purchased from Sigma Aldrich (St. Louis, Mo., USA). Sodium dihydrogen phosphate, 6-hydroxy-2,5,7,8-tetramethylchroman-2-carboxylic acid (trolox), 2,20-azobis (2-methylpropionamidine) dihydrochloride (AAPH) were purchased from Sapphire Bioscience (Redfern, NSW, Australia).

### Lactic Acid Bacteria

Lactic acid bacteria used during fermentation were selected from one or more of:

LP: Lactobacillus plantarum ATCC8014;

LGG: Lactobacillus rhamnosus ATCC53103;

B1: Lactobacillus plantarum isolated from broccoli deposited under V17/021731 on 25 Sep. 2017 at the National Measurement Institute Australia;

B2: Lactobacillus plantarum isolated from broccoli deposited under V17/021732 on 25 Sep. 2017 at the National Measurement Institute Australia;

B3: Lactobacillus plantarum isolated from broccoli deposited under V17/021733 on 25 Sep. 2017 at the National Measurement Institute Australia;

B4: Lactobacillus plantarum isolated from broccoli deposited under V17/021734 on 25 Sep. 2017 at the National Measurement Institute Australia;

B5: Lactobacillus plantarum isolated from broccoli deposited under V17/021735 on 25 Sep. 2017 at the National Measurement Institute Australia;

BF1: Leuconostoc mesenteroides isolated from broccoli puree deposited under V17/021729 on 25 Sep. 2017 at the National Measurement Institute Australia;

BF2: Leuconostoc mesenteroides isolated from broccoli puree BF2 deposited under V17/021730 on 25 Sep. 2017 at the National Measurement Institute Australia;

BP: pooled BF1, BF2; and

LAB: pooled B1, B2, B3, B4 and B5.

BF1 and BF2 were identified as Leuconostoc mesenteroides via a 16s-RNA sequence (Australian Genome Research Facility; data not shown). B1 to B5 were identified as Lactobacillus plantarum based on 16S-RNA sequence. The identity of all the isolates were confirmed by whole genome sequence analysis.

Isolation of Lactic Acid Bacteria from Broccoli and Broccoli Puree

The above Lactobacillus plantarum B1, B2, B3, B4 and B5 were isolated from broccoli leaves and stem. The leaves and stem were washed with water and homogenised with added peptone saline using a stomacher. The soaking solution was serially diluted and spread plated on De Man, Rogosa and Sharpe (MRS) agar. The plates were incubated under anaerobic condition for 48 to 72 hrs at 37° C. for isolating presumptive mesophilic lactic acid bacteria. Based on different colonial morphology on MRS plates, colonies were isolated, cultivated in MRS broth, screened using staining and biochemical characterisation techniques, and kept frozen with glycerol at −80° C. The isolates were identified at species level using 16s RNA sequencing at AGRF.

For the isolation of Leuconostoc mesenteroides BF1 and BF2, broccoli floret puree was used after serial dilution instead of the suspension described above for the isolation from broccoli leaves.

### Preparation of Starter Cultures

The lactic acid bacteria strains, Leuconostoc mesenteroides and Lactobacillus plantarum, were isolated from broccoli and identified by Australian Genome Research Facility Ltd. To obtain the primary culture, lactic acid bacteria cultures which were stored at −80° C. were inoculated into 10 mL of MRS broth (Oxoid, Victoria, Australia) and incubated at 30° C. for 24 h to obtain an initial biomass of 8 log colony-forming units per milliliter (CFU/mL). Two mL of each primary inoculum was inoculated into 200 mL of MRS broth and incubated for 24 hrs at 30° C. The cultures were collected by centrifugation at 2000 g for 15 min at 4° C., washed twice with sterile phosphate buffer saline (PBS), and all the Lactobacillus plantarum cultures were mixed together and all the Leuconostoc mesenteroides cultures were mixed together. The two culture suspensions were diluted to 10 log CFU/ml and were mixed at the same volumetric proportion and stored with glycerol at −80° C. until use as a mixed starter culture for broccoli fermentation.

### Fermentation Method

Broccoli (Brassica oleracea L. ssp. Italic; 30 kg) florets were cut approximately 2 cm from the crown, shredded to smaller pieces and, were macerated with Milli-Q water in ratio of 3:2 for 1 min using magic bullet blender. The broccoli slurry, was mixed well and placed into sterile plastic bottles (200 mL) with screw lids. Each bottle of broccoli puree (200 mL) was inoculated with the prepared starter culture at an initial concentration of 8 log CFU/g. The fermentation experiment was carried out in 48 bottles in parallel at 30° C., until a pH value of about 4.0 was reached (Day 4). After the fermentation phase was completed, 3 samples were taken out as the Day 0 storage samples, the other samples were separated to two lots for the storage experiments: one lot was stored in a refrigerator (4° C.) and another stored in room thermostated at 25° C. Samples were periodically taken over 12 weeks for microbiological, physicochemical and phytochemical analyses. The fermented broccoli puree was compared with raw broccoli puree which was stored at −20° C. after homogenization and puree samples incubated for the same period of time as the fermented samples without inoculation by LAB.

### Sampling

For time course experiments, sampling was performed at days 10, 20, 30, 40, 50, 60, 70, 80, and 90, and on days 14, 28, 42, 56, 70 and 84 for samples stored at 25° C. and 4° C., respectively. Sampling was performed in triplicate with color measured on the surface and pH measured immediately after opening the fermentation bottles. Thereafter, samples were taken for microbiological analysis and titratable acidity analysis. The remaining material was separated into two parts, the first portion was frozen and freeze dried, ground to fine powder and stored in a desiccator for further analyses, and the second part was frozen and kept at −20° C. until glucoraphanin and sulforaphane analyses.

### Microbiological Analysis

For microbial analysis, three different media were used to measure CFU per g broccoli puree of the different microorganisms; the plate counts for total lactic acid bacteria on DeMan-Rogosa-Sharp (MRS) agar, for total enterobacteria on violet red bile glucose agar (VRBGA), and the yeasts and mould on potato dextrose agar (PDA). For each sample, serial dilution of the broccoli suspension in sterilized peptone saline diluent were made and 0.1 mL of the dilutions were plated onto agar plates in duplicates. After aerobic incubation at 25° C. for 72 h (PDA), 37° C. for 24 h (VRBGA), and anaerobic incubation at 30° C. for 72 h (MRS), respectively, the CFU were counted.

### Determination of pH and Titratable Acidity

The pH value was determined directly in fermentation bottles containing broccoli puree by a pH meter (PHM240, MeterLab). Titratable acidity (TA) of broccoli samples was measured with an Automatic titrator (Titralab 854 titration manager, Radiometric Analytical, France). In brief, diluted broccoli puree (10 mL) was titrated using 0.1 M NaOH to the end point pH=8.1 and the result obtained was expressed as gram equivalent of lactic acid per liter of sample in accordance with the following equation:

$$TA ( g / L ) = \frac{[ v \times acid factor \times 1000 ]}{sample volume}$$

where, v is titer volume of NaOH. The acid factor for lactic acid is 0.009.

### Total Protein and Color Analyses

The total protein content of broccoli samples was determined as total nitrogen content multiplied by 6.25. Total nitrogen content of broccoli was analyzed using a Dumas combustion method with LECO TruMac apparatus (LECO Corporation, Michigan, USA). The color indexes (L, a, b) of fermented broccoli sample were determined using a Chroma meter CR-200 tristimulus colorimeter (Minolta, Osaka, Japan). The color values obtained were expressed as lightness/darkness (as L*), redness/greenness (a*) and yellow/blueness (b*). The total color difference (ΔE) was calculated according to the following equation:

ΔE=[(L*−L₀)²+(a*−a₀)²+(b*−b₀)²]^{1/2}

where, L₀, a₀, b₀ are color values of fresh unfermented broccoli.

### Determination of Total Polyphenol Content

The total phenolic content (TPC) was measured spectrophotometrically using the Folin-Ciocalteu colorimetric method (Singleton and Rossi, 1965) with modifications. Briefly, 50 mg of broccoli powder was suspended in 10 mL of acidified (1% HCl) methanol/water (70:30, v/v) solution and extracted in ultrasonic bath (IDK technology Pty Ltd, VIC, Australia) for 8 min. The extracts were kept for 16 h at 4° C. and filtered with 0.2 μM filter and stored at 4° C. until analysis. 1 mL of 0.2 N Folin-Ciocalteu reagent, 800 μL of sodium carbonate solution (7.5% p/v) and 180 μL Milli-Q grade water were added to the extract (20 μL). After 1 h of incubation in the dark at 37° C., the absorbance was measured at 765 nm in triplicates using a spectrophotometer (UV-1700 Pharma Spec, SHIMADZU). Gallic acid was used as a standard and TPC was expressed as the gallic acid equivalent (GAE) in mg per 100 g of fresh weight (mg GAE/100 g FW) based on a standard curve developed using known concentrations of gallic acid.

### Oxygen Radical Absorbance Capacity Assay

Freeze-dried broccoli powder (10 mg) was suspended in 10 mL of methanol/water (80:20, v/v), the extraction solvent. The slurry was extracted at 650 rpm on a Heidolph Multi-Reax (John Morris Scientific, NSW, Australia) at room temperature for an hour. Then it was centrifuged at 25,000 g for 15 min in 4° C., the supernatant was collected, and was ready for analysis after 100× dilution with 75 mM potassium phosphate buffer (pH 7.4). ORAC analysis was conducted according to the procedure reported by Huang et al. (2002) with minor modifications. The assay was carried out in opaque 96-well plates (dark optical bottom, Waltham, Mass., USA). The assay reactants included 81.6 nM of fluorescein, 153 mM of AAPH, Trolox standard of different concentration (100, 50, 25, 12.5, and 6.25 μM), and 75 mM phosphate buffer as the blank. The reactants were added in the following order: 25 μL of diluted sample; either 25 μL of 75 mM phosphate buffer, 25 μL Trolox standard and 150 μL fluorescein. After adding the fluorescein, the plate was incubated at 37° C. for 10 min and then the AAPH (25 μL) was added. Immediately after addition of AAPH, the plate was placed in the fluorescence plate reader (BMG Labtech ClarioStar, Germany) and the fluorescence was measured every 3 min until it decreased to less than 5% of original fluorescence. The ORAC values were calculated as the area under the curve (AUC) and expressed as micromoles of trolox equivalent (TE) per gram dry weight of broccoli (μmol TE/g DW). Each sample was assayed triplicate.

### Sulforaphane Analysis

The extraction of sulforaphane from broccoli matrix was conducted following the methods of Li et al. (2012) with some modification. In brief, frozen broccoli (2 g) was mixed with 2 mL of Milli-Q water and vortexed for 1 min. Then 20 mL ethyl acetate was added to the slurry followed by sonication for 5 min and shaking for 20 min at 4° C. The slurry was then centrifuged at 15,000 g for 10 min, and the supernatant was collected. Then another 15 mL ethyl acetate was added to the precipitate to carry out the second extraction. Pooled extracts from each sample were evaporated to dryness with a vacuum spin dryer (SC250EXP, Thermo Fisher Scientific, CA, USA) at room temperature, and stored at −20° C. until analysis. The concentration of sulforaphane was determined using an Acquity™ Ultra Performance LC system (Waters Corporation, Milford, Mass., USA), which is equipped with a binary solvent delivery manager and a sample manger. Chromatographic separations were performed on a 2.1×50 mm, Acquity BEH C18 chromatography column. The mobile phase A and B were 0.1% formic acid in millique water and 0.1% formic acid in acetonitrile, respectively. The gradient elution system consisted of mobile phase A (0.1% formic acid in millique water) and B (0.1% formic acid in acetonitrile) and separation was achieved using the following gradient: 0-2 min, 10% B; 2-5 min, 20% B; 5-10 min, 10% B. The column temperature was kept constant at 30° C. The flow-rate was 0.350 mL/min and the injection volume was 54.

Prior to analysis, all samples were dissolved in 1 mL 30% acetonitrile, and filtered through a 0.22 μm membrane filter (Merk Millipore, Billerica, Mass., USA). The identification of each peak was based on the retention time and the chromatography of authentic standards. The concentrations of each compound were calculated according to a standard curve, and the results were expressed as micromoles per kilogram DW (μmol/kg DW) of broccoli.

### Glucoraphanin Analysis

The extraction of glucoraphanin from raw or fermented broccoli was carried out according to the method of Cai and Wang (2016) with some modification. Accordingly, to 2 g of frozen broccoli puree, 10 mL of boiling Milli-Q water was added, and the mixture was incubated for 5 min in a boiling water bath. It was then cooled and centrifuged at 15000×g for 15 min, and the supernatant was collected. The precipitate was extracted once more with 8 mL of boiling water. Pooled extracts from each sample were evaporated to dryness with a vacuum spin dryer (Speedvac SC250EXP, Thermo Fisher Scientific, CA, USA) at 3° C., and stored at −20° C. until analysis. The concentration of glucoraphanin was quantified using an Alliance HPLC instrument (Waters Corporation, Milford, Mass., USA) equipped with Photo Diode Array Detector 2998. A HPLC column—Luna® 3 μM Hydrophilic Interaction Liquid Chromatography (HILIC) 200° A (100×4.6 mm; Phenomenex, Torrance, Calif., USA) was used for the analysis at a column temperature of 25° C. The mobile phase consisted of an acetonitrile/water (85:15, v/v) with 30 mM Ammonium formate (solution A) and acetonitrile (solution B) with the following isocratic flow program: solution A 70%; solution B 30%. Other chromatographic conditions included a constant flow rate of 2.0 mL/min, an injection volume of 100 μL, a run time of 8 min, and detection wavelength of 235 nm. Prior to analysis, all samples were dissolved in 1 mL solvent A, and filtered through a 0.22 μm membrane filter (Merk Millipore, Billerica, Mass., USA). The identification of each peak was based on the retention time and the chromatography of an authentic glucoraphanin standard. The concentrations of glucoraphanin were calculated using a standard curve, and the results were expressed as micromoles glucoraphanin per kilogram DW (μmol/kg DW) of broccoli.

### Statistical Analysis

All experiments were conducted in triplicate and the results were expressed as mean values. A one-way analyses of variance (ANOVA) was applied to evaluate the significance of the differences among the mean values at 0.05 significance level (p<0.05). The statistical analysis was conducted using the statistical software, SPSS 16.0 for Windows (SPSS Inc., Chicago, Ill., USA).

### Example 2—Microbial Analysis of Lactic Acid Bacteria Fermented Broccoli Florets

The fermentation of broccoli puree was carried out as described in the fermentation section of Example 1. The counts of total lactic acid bacteria were lower for raw broccoli compared to inoculated broccoli as showed in Table 1. After 4 days of fermentation, the pH of the sample reached 4.04 and fermentation was stopped, and the fermented sample before storage experiments was taken as the Day 0 sample. It is clear from Table 1 and FIG. 1C that the counts of total lactic acid bacteria of the Day 0 sample were significantly increased (8 log CFU/g) compared to the raw broccoli. During the first two weeks of storage, the viable number of total lactic acid bacteria increased to the highest values of 9 log CFU/g for samples stored at both 25° C. and 4° C. (Table 1 and Table 2). During storage at 25° C., the total lactic acid bacteria counts increased to 9 log CFU/g at Day 10 and slowly declined during storage to 5 log CFU/g by Day 50, and declined further to almost undetectable level after Day 70. In contrast, the LAB count in the samples stored at 4° C. remained high (6 log CFU/g) even after storage for 84 days.

TABLE 1
Microbiological and physicochemical changes of fermented broccoli during the storage at room temperature (25° C.).

| | Microbial loads (Log CFU/g) | | | | | TP (mg/ | Color | | | |
|---|---|---|---|---|---|---|---|---|---|---|
| | MRS | PDA | VRBGA | pH | TA (g/L) | g, FW) | L | a | b | ΔE |
| Raw | 2.4 ± 0.2 | 2.5 ± 0.1 | 3.4 ± 0.1 | 6.33 ± 0.00 | 4.8 ± 0.2 | 26.9 ± 0.0 | 48.4 ± 0.4 | −13.2 ± 0.1 | 17.2 ± 0.2 | — |
| broccoli | | | | | | | | | | |
| Day 0 | 8.4 ± 0.2 | <1 | <1 | 4.04 ± 0.00 | 10.7 ± 0.7 | 29.6 ± 0.8 | 48.5 ± 0.7 | −2.1 ± 0.1 | 13.6 ± 0.6 | 11.7 |
| Days 10 | 9.4 ± 0.1 | <1 | <1 | 3.87 ± 0.02 | 14.4 ± 0.2 | 27.8 ± 0.8 | 47.7 ± 0.8 | −1.1 ± 0.2 | 12.2 ± 0.5 | 13.1 |
| Days 20 | 6.2 ± 0.3 | <1 | <1 | 3.76 ± 0.02 | 14.7 ± 0.2 | 30.5 ± 0.8 | 47.1 ± 0.5 | −1.1 ± 0.0 | 12.5 ± 0.2 | 13 |
| Days 30 | 6.2 ± 0.1 | <1 | <1 | 3.78 ± 0.00 | 15.1 ± 0.3 | 29.7 ± 1.2 | 47.2 ± 0.2 | −1.0 ± 0.1 | 10.9 ± 0.5 | 13.8 |
| Days 40 | 6.1 ± 0.4 | <1 | <1 | 3.79 ± 0.02 | 15.1 ± 0.4 | 28.8 ± 1.1 | 46.3 ± 0.5 | −0.8 ± 0.1 | 11.0 ± 0.9 | 14 |
| Days 50 | 5.1 ± 0.6 | <1 | <1 | 3.75 ± 0.00 | 15.2 ± 0.5 | 28.5 ± 0.1 | 45.8 ± 0.5 | −0.9 ± 0.1 | 11.0 ± 0.2 | 14 |
| Days 60 | 2.4 ± 0.1 | <1 | <1 | 3.76 ± 0.01 | 15.4 ± 0.3 | 27.3 ± 0.6 | 45.4 ± 0.1 | −0.9 ± 0.1 | 10.5 ± 0.1 | 14.3 |
| Days 70 | 1.5 ± 0.1 | <1 | <1 | 3.76 ± 0.01 | 15.7 ± 0.1 | 27.7 ± 0.2 | 45.3 ± 0.5 | −0.9 ± 0.1 | 9.9 ± 0.4 | 14.7 |
| Days 80 | <1 | <1 | <1 | 3.76 ± 0.01 | 15.7 ± 0.7 | 28.3 ± 0.2 | 45.9 ± 0.1 | −0.9 ± 0.1 | 9.7 ± 0.1 | 14.6 |
| Days 90 | <1 | <1 | <1 | 3.71 ± 0.01 | 15.7 ± 0.3 | 28.7 ± 0.4 | 45.0 ± 0.0 | −0.8 ± 0.2 | 9.3 ± 0.2 | 15.1 |

Each value was expressed as mean ± standard deviation (n = 3).
“—” not available.
MRS, de Man-Rogosa-Sharpe agar for LAB;
PDA, potato dextrose agar for total yeasts and moulds;
VRBGA, violet red bile glucose agar for Enterobacteriaceae;
TA, titratable acidity;
TP: total protein;
ΔE: total color difference.

TABLE 2
Microbiological and physicochemical changes of fermented broccoli during the storage at 4° C..

| | Microbial loads (Log CFU/g) | | | | | TP (mg/ | Color | | | |
|---|---|---|---|---|---|---|---|---|---|---|
| | MRS | PDA | VRBGA | pH | TA (g/L) | g, FW) | L | a | b | ΔE |
| Raw | 2.4 ± 0.2 | 2.5 ± 0.1 | 3.4 ± 0.1 | 6.33 ± 0.00 | 4.8 ± 0.2 | 26.9 ± 0.0 | 48.4 ± 0.4 | −13.2 ± 0.1 | 17.2 ± 0.2 | — |
| broccoli | | | | | | | | | | |
| Day 0 | 8.4 ± 0.2 | <1 | <1 | 4.04 ± 0.00 | 10.7 ± 0.7 | 29.6 ± 0.8 | 48.5 ± 0.7 | −2.1 ± 0.1 | 13.6 ± 0.6 | 11.7 |
| Days 14 | 9.0 ± 0.1 | <1 | <1 | 4.04 ± 0.03 | 12.6 ± 0.8 | 32.5 ± 1.2 | 47.2 ± 1.1 | −1.9 ± 0.5 | 12.4 ± 1.5 | 12.3 |
| Days 28 | 8.0 ± 0.1 | <1 | <1 | 3.95 ± 0.02 | 13.5 ± 0.8 | 32.0 ± 0.7 | 45.9 ± 0.7 | −2.2 ± 0.3 | 13.8 ± 2.5 | 11.8 |
| Days 42 | 7.6 ± 0.1 | <1 | <1 | 3.89 ± 0.03 | 13.8 ± 0.2 | 32.0 ± 0.8 | 46.7 ± 0.2 | −1.5 ± 0.1 | 12.6 ± 0.5 | 12.7 |
| Days 56 | 6.5 ± 0.4 | <1 | <1 | 3.89 ± 0.02 | 13.8 ± 0.5 | 29.9 ± 0.3 | 46.6 ± 0.4 | −1.7 ± 0.1 | 13.1 ± 0.5 | 12.4 |
| Days 70 | 6.3 ± 0.4 | <1 | <1 | 3.86 ± 0.01 | 13.7 ± 0.1 | 31.6 ± 0.2 | 46.7 ± 0.8 | −1.6 ± 0.2 | 12.2 ± 0.4 | 12.7 |
| Days 84 | 6.0 ± 0.8 | <1 | <1 | 3.85 ± 0.01 | 13.8 ± 0.1 | 32.0 ± 0.5 | 47.6 ± 0.9 | −1.9 ± 0.2 | 14.0 ± 0.6 | 11.8 |

Each value was expressed as mean ± standard deviation (n = 3).
“—” not available.
MRS, de Man-Rogosa-Sharpe agar for LAB;
PDA, potato dextrose agar for total yeasts and moulds;
VRBGA, violet red bile glucose agar for Enterobacteriaceae;
TA, titratable acidity;
TP: total protein;
ΔE: total color difference.

The total counts of yeast and moulds in the raw broccoli sample was 2 log CFU/g. The Enterobacteriaceae count in the raw broccoli with 3 log CFU/g. No fungi, moulds and enterobacteria were detected after fermentation or on the fermented samples after storage at both temperature conditions. No pathogenic and spoilage organisms were detected following fermentation and during storage. The results indicate that the fermentation process resulted in a safe and stable product with undetectable level of potentially pathogenic eneterobacteriaceae and spoilage yeast and mould, which maintained high levels of total lactic acid bacteria when stored at 4° C. There are ˜10⁶ CFU/g lactic acid bacteria after ˜3 months at 4° C.

### Example 3—Assessment of pH and Titratable Acidity after Storage of Lactic Acid Bacteria Fermented Broccoli Florets

The pH and titratable acidity (TA) of raw broccoli, fermented broccoli and fermented broccoli after storage at 25° C. and 4° C. was analyzed as described in Example 1. The determination of TA was used to estimate the amount of lactic acid and acetic acid, the main acids produced by lactic acid bacteria, during fermentation. During fermentation, the acids produced by the lactic acid bacteria decrease the pH of the sample. As shown in Table 1, the TA was increased to 10.7 g/L in Day 0 samples. When stored in 25° C., the pH was decreased to 3.87 during storage after 10 days, along with the significantly increased values of TA which reached 14.4 g/L (p<0.05; see Table 1). The results indicate that there were still substrates present for lactic acid bacteria to consume and further produce acid during the early days of storage. Neither the pH nor TA value were significantly changed during the remaining storage period (Table 1).

Decreasing the temperature to 4° C. reduced the rate of decrease of pH and TA in the stored samples due to the decreased activity of the lactic acid bacteria at the lower temperature (see Table 2). After nearly 3 months storage at 4° C., the pH was 3.85 and the TA value was 13.7 g/L.

### Example 4—Assessment of Broccoli Maceration and Fermentation on the Conversion of Glucoraphanin into Sulforaphane

Broccoli florets were cut into small pieces, mixed with water at 3:2 broccoli:water ratio and the mixture was macerated into a puree using a blender. Puree samples (200 gm) were aliquoted into sterile plastic bottles. The samples were inoculated at 10⁸ CFU/gm with pooled culture of lactic acid bacteria (Leuconostoc mesenteroides and Lactobacillus plantarum) isolated from Australian broccoli. Samples were incubated in a water bath maintained at 30° C. until the pH dropped to ˜4.0, which was attained after four days of fermentation. Control non-inoculated samples were immediately frozen after maceration. A second set of non-inoculated control samples, to which sodium benzoate was added to inhibit microbial growth, were incubated with the inoculated samples at 30° C. for four days until the fermentation of the inoculated samples was completed. Experiments were conducted in triplicate. All samples were kept frozen until sulforaphane and glucoraphanin analysis. As shown in FIG. 1B and Table 3 maceration followed by fermentation increased the sulforaphane yield compared to just maceration and incubation alone.

TABLE 3
Effects of maceration and fermentation on sulforaphane content in
broccoli puree.

| 25° C. | SF (mg/kg, DW) | | 4° C. | SE (mg/Kg, DW) | |
|---|---|---|---|---|---|
| Raw material | 149.8 | ± 12.4 | Raw material | 149.8 | ± 12.4 |
| Control | 86.8 | ± 0.6 | Control | 86.8 | ± 0.6 |
| incubated | | | incubated | | |
| 0 days | 278.4 | ± 1.8 | 0 days | 278.4 | ± 1.8 |
| 10 days | 189 | ± 8.8 | 14 days | 288.6 | ± 3.1 |
| 20 days | 136.6 | ± 6.2 | 28 days | 218.8 | ± 4.3 |
| 30 days | 122.2 | ± 12.2 | 42 days | 199.4 | ± 14.7 |
| 40 days | 116.3 | ± 5.0 | 56 days | 190 | ± 7.1 |
| 50 days | 112.3 | ± 4.0 | 70 days | 190.8 | ± 10.7 |
| 60 days | 111.9 | ± 11.0 | 84 days | 179.6 | ± 10.2 |

| | | |
|---|---|---|
| 70 days | 108.8 | ± 15.8 |
| 80 days | 102.6 | ± 14.7 |
| 90 days | 87.6 | ± 3.7 |

### Example 5—Assessment of Total Protein Content and Color after Storage of Lactic Acid Bacteria Fermented Broccoli Florets

The total protein content and color of lactic acid fermented broccoli florets after fermentation was assessed as described above in the methods section. Compared to raw broccoli (26.9±0.03), the total protein content of fermented broccoli was significantly increased (29.6±0.8 mg/g; p<0.05). This could be due to the high number of lactic acid bacteria inoculated into the sample and the growth during fermentation and protein synthesis by the lactic acid bacteria. The total protein content stayed stable during storage both at 25° C. and 4° C. (Table 1 and Table 2), with no significant difference between samples.

The color values (L, a, b) and the total color difference (ΔE) of broccoli samples are summarized in Table 1 and Table 2. As presented in Table 1 and Table 2, significant differences in the color parameters and the total color difference value (ΔE) were recorded between raw and fermented samples. The L* value (lightness) did not change significantly, whereas a* (greenness) and b* (yellowness) values decreased after the fermentation of broccoli puree. The decrease in a* and b* values may be attributed to the degradation in the color pigmented compounds, such as chlorophyll which would convert to pheophytins under the low pH. The high ΔE value (12.5) of Day 0 sample indicate that the color of broccoli puree was significantly changed after fermentation, which was visually noticeable. During storage (Table 1 and Table 2) there was no significant change in the ΔE value in neither 25° C. nor 4° C. samples.

Broccoli after fermentation with LAB+BP (Lactobacillus plantarums B1, B2, B3, B4, B5 and Leuconostoc mesenteroides BF1, BF2 isolated from broccoli) had a brighter, more intense green color more similar in color to raw macerated broccoli compared to broccoli fermented with LAB only (the Lactobacillus plantarums isolated from broccoli (B1, B2, B3, B4, B5)).

### Example 6—Changes of Total Phenolic Content and Antioxidant Activity of Lactic Acid Bacteria in Fermented Broccoli Florets

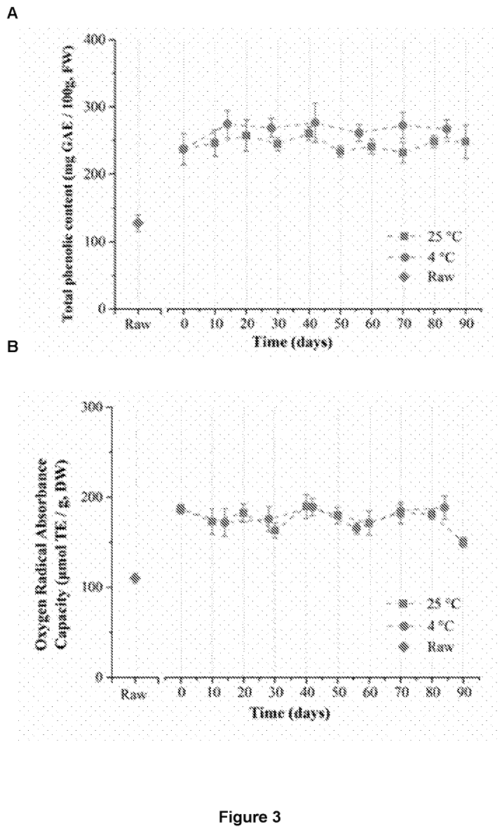

The total phenolic content (TPC) and antioxidant activity of lactic acid fermented broccoli florets after fermentation was assessed as described above in the methods section. The TPC of raw broccoli was 127.6±12.4 mg GAE/100 g (FIG. 3A) of fresh weight. The values of TPC on Day 0 significantly increased to 236.9±23.4 mg GAE/100 g (p<0.05) compared to raw broccoli. There was no significant difference between samples stored at 25° C. and 4° C. in the TPC after storage (FIG. 3A). When stored at 25° C., the value of TPC in fermented broccoli was 246.2±19.3 mg GAE/100 g on Days 10, and 248.1±25.0 mg GAE/100 g on Days 90. When stored at 4° C., the values of TPC was 274.1±20.2 and 267.2±3.3 mg GAE/100 g for Days 14 and Days 84, respectively.

The antioxidant activities of sample expressed as ORAC values are shown in FIG. 3B. The ORAC value of the raw sample was 110.1±0.05 μmol TE/g. Fermentation significantly increased the ORAC value by ˜70% to 186.9±3.3 μmol TE/g when compared to raw broccoli. This result suggested that antioxidant compounds may have increased during fermentation and was consistent with the change in TPC after fermentation.

During storage, the antioxidant activity of fermented broccoli did not change significantly. As shown in FIG. 3B, when stored at 25° C., the values of ORAC at Days 10 and Days 90 were 173.0±14.4 and 150±5.5 μmol TE/g, respectively. Similar results were obtained for samples stored at 4° C. The ORAC value was 172.0±15.5 μmol TE/g at the beginning of storage, which increased to a maximum value of (188.7±12.9 μmol TE/g) after storage.

### Example 7—Assessment of Fermentation Time for Different Combinations of Lactic Acid Bacteria

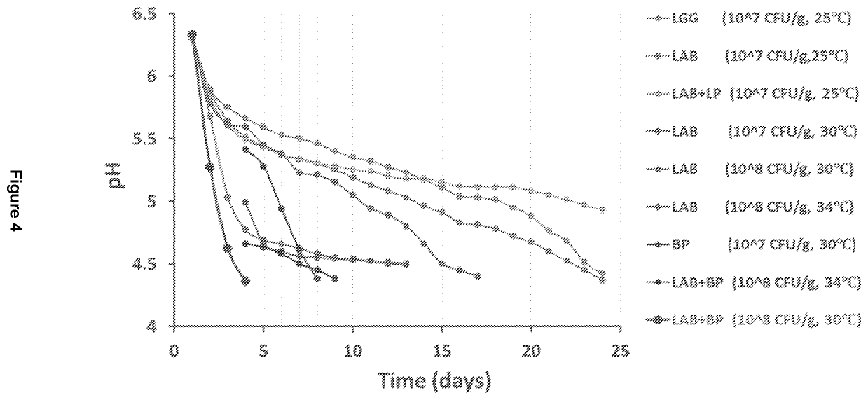

Macerated broccoli was prepared as described above in the methods section with a broccoli to water ratio of 3:2 and a maceration time of 1 min. The broccoli material was inoculated with either 10⁷ CFU/g or 10⁸ CFU/g with one of: LGG, LAB (Lactobacillus plantarum (B1, B2, B3, B4, B5) isolated from Australian broccoli, LAB+LP (Lactobacillus plantarum isolated from broccoli and Lactobacillus sp. ATCC 8014), BP (Leuconostoc mesenteroides isolated from broccoli), LAB+BP (a mixture of the two groups as described in the methods sections) and fermented at either 25° C., 30° C. or 34° C. to reach a target pH of 4.4. As shown in FIG. 4 the addition of lactic acid bacteria isolated from broccoli and/or broccoli puree significantly reduced the time taken for the fermentation with the combination of LAB+BP reaching a pH of 4.4 after fermenting for about 4 days. An example composition of fermented broccoli product is shown in Table 4.

TABLE 4
Composition of the fermented broccoli product.

| Quality attributes | Value |
|---|---|
| Total fibre | ~29.5 g/100 gdw |
| ORAC antioxidant capacity | 18695 μmol TE/100 gdw |
| Total polyphenol content | 2369 mg GAE/100 gdw |
| Total titratable acidity | 1.1% lactic acid equiv. |
| Lactic acid bacteria count | ~10⁸ CFU/gm |
| Total protein | 30 g/100 gdw |
| Broccoli to water ratio in puree | 3 to 2 |
| by mass | |

### Example 8—Effect of Storage on Sulforaphane Content of Fermented Broccoli

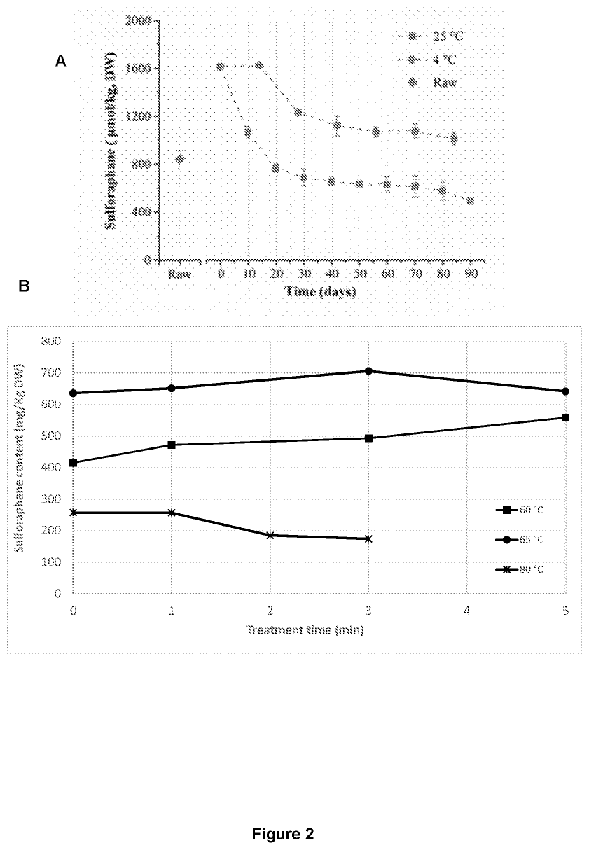

FIG. 2A shows the effects of storage at 4 and 25° C. on sulforaphane content of fermented broccoli puree. As can be seen in the FIG. 2A, the sulforaphane content of samples stored at 25° C. dramatically decreased to 770.7±34.9 μmol/kg (a 52% loss) after 20 days storage, followed by a slower decline during the rest of the storage period, reaching a total loss of 69.5%. Interestingly, no statistically significant change in sulforaphane content was observed during the first 2 weeks of storage of fermented broccoli samples at 4° C. A significant decrease of ˜23.7% occurred during the subsequent two weeks followed by a slow degradation during the rest of the storage period. At the end of the storage (Day 84), the sulforaphane content was 1012.9±57.6 μmol/kg in samples stored at 4° C., making the total loss of sulforaphane ˜37.4% compared to the Day 0 samples. The sulforaphane content during the first two weeks of storage was maintained perhaps due to simultaneous production and degradation of sulforaphane since some decrease in glucoraphanin content was observed in the 4° C. stored samples over the same period.

### Example 9—Effect of Fermentation and Storage on Glucoraphanin Content

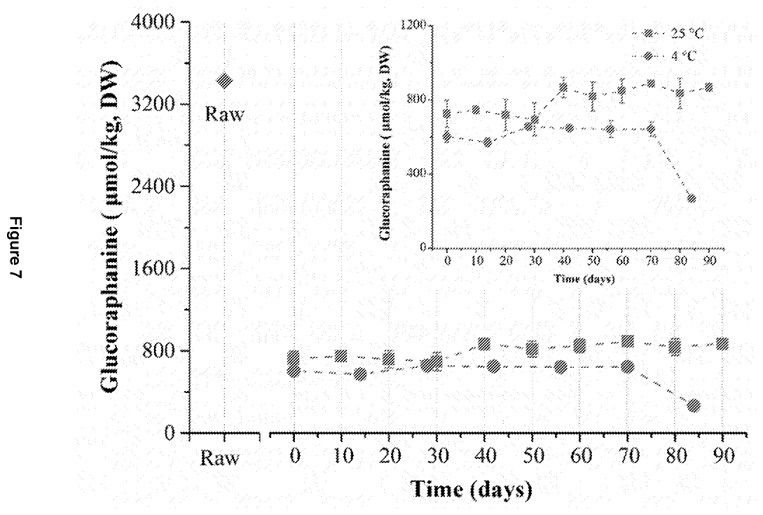

FIG. 7 shows the effect of maceration and fermentation on glucoraphanin content and its stability during storage at 4° C. and 25° C. The glucoraphanin content of raw broccoli was 3423.7±39.7 μmol/kg (FIG. 7), After fermentation, the glucoraphanin content sharply decreased to 712.4±64.2 μmol/kg (Day 0 sample). Glucoraphanin is relatively stable in intact tissue and the degradation in this case can be attributed to myrosinase catalyzed hydrolysis due to increased enzyme-substrate interaction in the macerated tissue during fermentation. The period of sharp decrease in glucoraphanin coincided with the fermentation period.

No significant change in glucoraphanin content was observed in fermented samples during storage at 25° C. and 4° C. However, slightly higher glucoraphanin content was observed in samples stored at 25° C. This could be related to the faster decline in pH of the samples stored at 25° C. (pH 3.87 at the second time point) compared to samples stored at 4° C. (pH 4.04 at the second time point). The optimal pH for myrosinase catalyzed hydrolysis of glucoraphanin ranges from 5 to 6 decreasing to the lowest value at pH 3.0 (Dosz & Jeffery, 2013). The relatively higher pH of the samples stored a 4° C. may have contributed to the slightly higher degradation of glucoraphanin during storage at 4° C. compared to 25° C.

### Example 10—Assessment of Heat Treatment Conditions to Maximise Conversion of Glucoraphanin into Sulforaphane in Broccoli Matrix

Broccoli florets packed in retort pouches were subjected to thermal processing at temperatures ranging from 60° C. to 80° C. and treatment times of 0 to 5 minutes. The treatment involved pre-heating to the experimental temperature in a water bath maintained at 5° C. higher than the experimental temperature followed by incubation in a second water bath maintained at the experimental temperature. Following thermal treatment, samples were cooled in ice-water and were macerated with water added at 2:3 water to broccoli ratio as described above. The macerated samples were incubated for 1 hr at 30° C. and kept frozen until sulforaphane analysis. Results are shown in FIG. 2B and Table 5. As shown in Table 5 pre-heating the sample at 60° C., 65° C. or 80° C. followed by maceration increased the sulforaphane yield relative to raw broccoli floret which was macerated without pre-heating.

TABLE 5
Effects of heat treatment on sulforaphane production in broccoli matrix.

| | Heat | | | |
|---|---|---|---|---|
| | treatment | | | |
| | time | Sulforaphane | Sulforaphane | Sulforaphane |
| Temperature | (minute) | (μmol/kg, DW) | (mg/kg, DW) | (mg/g, DW) |
| Raw | — | 817.5 ± 9.29 | 145 ± 1.6 | 0.145 ± 0.002 |
| broccoli | | | | |
| floret | | | | |
| 60° C. | 0 | 2343.5 ± 124.1 | 415.5 ± 22.0 | 0.415 ± 0.022 |
| | 1 | 2661.5 ± 10.9 | 471.9 ± 1.9 | 0.472 ± 0.002 |
| | 3 | 2780.9 ± 270.7 | 493.0 ± 48.0 | 0.493 ± 0.048 |
| | 5 | 3147.6 ± 148 | 558.1 ± 26.2 | 0.558 ± 0.026 |
| 65° C. | 0 | 3585.9 ± 119.2 | 635.8 ± 21.1 | 0.636 ± 0.021 |
| | 1 | 3673 ± 144.8 | 651.2 ± 25.7 | 0.651 ± 0.026 |
| | 3 | 3983.4 ± 30.5 | 706.3 ± 5.4 | 0.706 ± 0.005 |
| | 5 | 3620.1 ± 240.7 | 641.8 ± 42.7 | 0.642 ± 0.043 |
| 80° C. | 0 | 1451.5 ± 43.5 | 257.3 ± 7.7 | 0.257 ± 0.008 |
| | 1 | 1446.8 ± 17.5 | 256.5 ± 3.1 | 0.257 ± 0.003 |
| | 2 | 1043.1 ± 94.2 | 184.9 ± 16.7 | 0.185 ± 0.017 |
| | 3 | 981.2 ± 35.1 | 174 ± 6.2 | 0.174 ± 0.006 |

### Example 11—Assessment of Preheating Prior to Lactic Acid Bacterial Fermentation on the Sulforaphane Content of Broccoli

This study evaluated the impact of mild preheating treatment of broccoli florets to inactivate the Epithiospecifier protein (ESP) combined with lactic acid bacteria on sulforaphane content of broccoli puree.

### Materials

Broccoli (cv. ‘Viper’) was purchased from a local supermarket (Coles, Werribee South, VIC, Australia). DeMan-Rogosa-Sharp (MRS) broth (1823477, CM0359, Oxoid) was purchased from Thermo Fisher Scientific (Australia). DL-Sulforaphane was purchased from Sigma-Aldrich (St. Louis, Mo., USA). All the other chemical and biochemical reagents were analytical grade or higher and were purchased from local chemical vendors.

### Experiments to Optimize the Mild Pre-Heating Conditions to Maximize Sulforaphane Yield

Broccoli florets were cut at approximately 2 cm below the head, and each 30 g of randomly mixed broccoli florets were used in the pre-heating experiments. Two types of pre-heating experiments were conducted; in-pack processing and direct water blanching. In the case of the in-pack experiments, broccoli florets were packed in retort pouches (Caspak Australia, Melbourne), sealed and pre-heated for various time points in a thermostated water batch maintained at 60° C., 65° C. and 80° C. The temperature of the broccoli samples at the slowest heating point was measured by using a thermometer. Time 0 was defined as the time for the core temperature to reach the designated experimental temperature. The treatment time were 0, 1, 3, and 5 min for 60° C. and 65° C. and 0, 1, 2, 3 min for 80° C. With the direct water-blanching experiments, the broccoli florets were immersed in Milli-Q water in a glass beaker that was heated in a thermosated water-bath. The direct water blanching experiments were conducted at 60° C. and 65° C. The temperature of the broccoli samples was continuously measured using a thermometer and timing started once the temperature at the slowest heating point attained the designated experimental temperature as described above. All thermal treatment experiments were carried out in triplicate. Unheated broccoli florets were used as controls. Immediately following the heat treatment, the samples were cooled in ice water and were homogenized with Milli-Q water in ratio of 3 parts broccoli to 2 parts of water for 1 min using a kitchen scale magic bullet blender (Nutribullet pro 900 series, LLC, USA). The homogenized samples were incubated in the dark for 4 h at 25° C. to allow the enzymatic hydrolysis of glucoraphanin After incubation, all the samples were frozen in −20° C. until sulforaphane analysis.

### Preparation of Starter Cultures

Pooled cultures of Leuconostoc mesenteroides (BF1, BF2) and Lactobacillus plantarum (B1, B2, B3, B4, B5) isolated from broccoli as described in the methods in Example 1. were used in the fermentation experiments. The lactic acid bacteria stock cultures, which were stored at −80° C., were activated by inoculation into 10 mL MRS broth (Oxoid, Victoria, Australia) and incubation at 30° C. for 24 hours to get the primary inoculum. 2 mL of the primary cultures were inoculated into 200 mL of MRS broth to obtain the secondary cultures. After 24 h incubation, the 6 secondary cultures were centrifuged, washed twice with sterile phosphate buffer saline (PBS) and each of the culture was resuspended in Milli-Q water at a concentration of 10 log colony-forming units per millilitre (CFU/mL) to obtain an initial biomass of 8 log CFU/mL in 100 gm broccoli puree samples. The L. plantarum cultures were mixed with the L. mesenteroides cultures at 1:1 proportion prior to inoculation into the broccoli puree samples.

### Sample Preparation

Broccoli florets were cut at approximately 2 cm below the crown and were separated into two lots; heat treated and non-treated. After heat treatment at the optimal condition selected based on the results of the experiments as described above, the samples were cooled in ice-water, shredded and homogenized with Milli-Q water in ratio of 3:2 for 1 min using a kitchen scale magic bullet blender (Nutribullet pro 900 series, LLC, USA). The non-treated broccoli were also homogenized in a similar way. The broccoli puree, after mixing well, was aliquoted into sterile plastic containers (100 mL) with screw lids (Technoplast Australia) for further experiments.

### Fermentation

Broccoli puree samples (pre-heated and untreated) were inoculated with the LAB culture prepared as described above in this example. Preheating of broccoli florets was conducted in-pack at 65° C. for 3 min based on the result of the experiment to optimise the pre-heating condition. In order to evaluate the impact of acidification without fermentation on conversion of glucoraphanin into sulforaphane, acidification experiments were conducted on pre-heated and untreated broccoli puree using glucono-delta-lactone (GDL) to attain the pH of the fermented broccoli puree. Preheated broccoli puree and untreated broccoli puree without further treatment were used as controls.

For the fermentation experiment, each broccoli puree sample was inoculated with the prepared starter culture at an initial level of 8 log CFU/g. The fermentation experiment was carried out at 30° C. until the pH reached ˜4.0 after 15 hrs of incubation. Once the fermentation was completed, 3 samples (day 0 samples) of each fermented group were taken and stored at −20° C. until analysis. The rest of the ferments were randomly separated into two lots for the storage trials: one lot was stored under refrigerated condition (4° C.) and the second lot was stored at 25° C. for the assessment of the sulforaphane stability of the samples after 14 days storage. Similarly, the untreated broccoli puree, preheated broccoli puree and the preheated-GDL treated broccoli puree were also sampled at time zero and stored at 25 and 4° C. for the 14 days storage trials. After 14 days storage, all the samples were frozen and kept at −20° C. until sulforaphane analyses.

Sulforaphane Analysis and Statistical Analysis Was performed as described in Example 1.

### Optimization of Heat Treatment Conditions for Improving Sulforaphane Yield

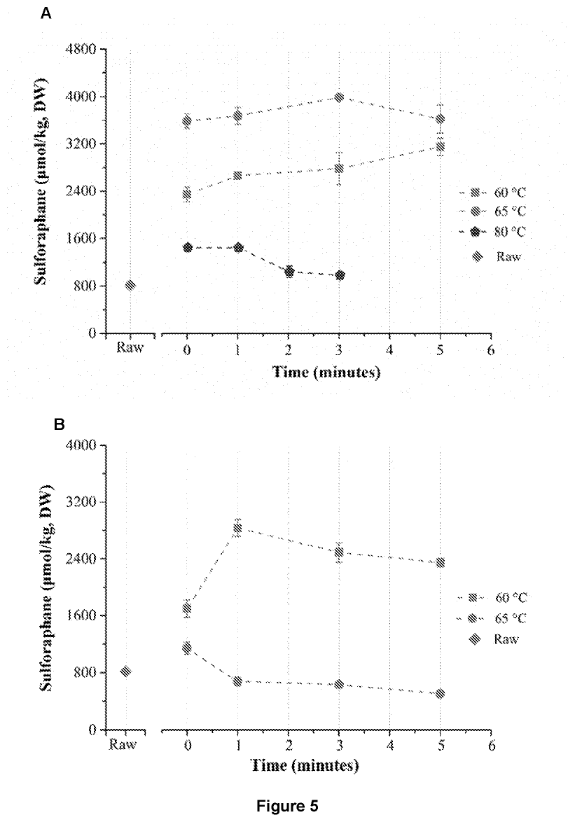

The influence of heat treatment on the formation of sulforaphane of the heated-in-pack broccoli florets at three different temperatures (60, 65 and 80° C.) for various processing times (0, 1, 3 and 5 min for 60 or 65° C.; 0, 1, 2 and 3 min for 80° C.) are shown in FIG. 5A. The results showed that compared to the raw broccoli the sulforaphane yield increased in all of the heat treated samples. Time 0 designate samples that were heated until their core reached the experimental temperature.

As shown in FIG. 5A, an increase in sulforaphane yield occurred when the packed broccoli samples were heated at 60° C. for 0, 1, 3, and 5 min. The concentration of sulforaphane in these samples were 2343.5±124.1, 2661.5±10.9, 2780.9±270.8, and 3147.7±148.0 μmol/kg DW, respectively. On the other hand, when broccoli was processed at 65° C., the sulforaphane yield initially increased with processing time from 3585.9±119.2 (0 min) to the highest value of 3983.4±30.5 μmol/kg DW (3 min). Further increase in treatment time resulted in lower yield with the lowest value of 3620.1±240.7 μmol/kg observed after 5 min treatment time. In contrast to treatments at 60 and 65° C., for samples that were processed at 80° C., a steady decrease in sulforaphane yield was observed with longer treatment times; with sulforaphane content of 1451.5±43.5, 1446.8±17.5, 1043.1±94.2, and 981.2±35.1 μmol/kg DW after 0 min, 1 min, 2 min and 3 min treatment respectively. Overall, the highest yield of sulforaphane (3983.4±30.5 μmol/kg) for in-pack treatment of broccoli was obtained for samples pre-heated at 65° C. for 3 min, which is ˜5 fold higher than raw broccoli (817.5±9.3 μmol/kg DW). In contrast, heating broccoli directly in water, generally resulted in a lower yield of sulforaphane compared to in-pack processing as shown in FIG. 5B. For direct water blanching at 60° C., the sulforaphane yield increased with treatment time from 1698.00±121.9 μmol/kg DW (0 min), to 2833.3±118.6 μmol/kg DW (1 min) and then steadily decreased to the lowest value of 2345.8±57.7 μmol/kg DW for 5 min treatment at 60° C. A sharp drop in sulforaphane yield compared to 60° C. was observed when samples were blanched at 65° C. The sulforaphane yield was 503.7±23.8 μmol/kg DW of broccoli after 5 min thermal treatment at 65° C., which was even lower than the value obtained for raw broccoli. The reason could be the leaching of glucoraphanin into the blanching water resulting in low yield of sulforaphane. For direct water blanching, the optimum treatment temperature for maximizing sulforaphane yield was 60° C. compared to 65° C. for the in-pack processing.

In this study, the highest yield of sulforaphane was obtained for broccoli florets processed in-pack for 3 min at 65° C., indicating that the condition favors the inactivation of ESP to a larger extent while maintaining sufficient myrosinase activity resulting in optimal conversion into sulforaphane. Under this condition, it seems that most of the extractable glucoraphanin is converted to sulforaphane assuming 1 to 1 conversion, since the glucoraphanin content of the broccoli samples were determined to be 3423.7±39.7 μmol/kg DW.

The observation that the exposure of the heat-treated broccoli to fermentation resulted in higher levels of sulforaphane than would be predicted from the level of extractable glucoraphanin from raw broccoli suggests heat-treatment may have increased the accessibility of glucoraphanin to myrosinase, resulting in higher sulforaphane yield than would be expected based on the quantifiable amount of glucoraphanin present in the untreated broccoli.

Less sulforaphane yield was obtained for broccoli florets directly blanched in water, most probably due to leaching into the blanching water, since glucoraphanin is soluble in water. It is also interesting to note that when broccoli florets were heated directly in water, the maximum amount of sulforaphane was obtained by heating at 60° C. for 1 min compared to 65° C. for 3 min when heat treatment of broccoli florets was done in-pack. This may be due to the higher leaching rate into the blanching water at 65° C. which counteracted the effects of higher level of inactivation of ESP at 65° C.

### The Effect of LAB Fermentation and Chemical Acidification on Sulforaphane Yield

Broccoli florets were pre-heated in-pack at the best treatment condition selected above (65° C., 3 min). Samples were then either fermentation by lactic acid bacteria or acidified using the acidulant (GDL). Consistent with the pre-treatment experiments, the sulforaphane value of broccoli significantly increased (p<0.05) after the heat treatment; with 806.2±7.0 μmol/kg DW and 3536.0±136.9 μmol/kg DW of sulforaphane yield for raw and pre-heated broccoli, respectively. The value of 3536 μmol/kg DW obtained with this separate batch of broccoli preheated prior to fermentation is of the same order obtained when a different batch of broccoli was used, where 3983 μmol/kg DW was obtained indicating slight batch to batch variation.

As shown in Table 6, after the fermentation, the sulforaphane content of broccoli samples varied depending on the treatment of the broccoli prior to fermentation. The sulforaphane content of raw broccoli puree after fermentation (1617.4±10.2 μmol/kg DW) was approximately twice the sulforaphane content of raw broccoli puree. Pre-heating of broccoli prior to pureeing resulted in much higher increase in sulforaphane content after fermentation. The sulforaphane content of preheated-fermented broccoli (13121.3±440.8 μmol/kg DW) was about 8 times of the raw-fermented broccoli puree. The observed sulforaphane yield after the combined preheating-fermentation treatment is much higher than what would be expected based on the quantifiable amount of glucoraphanin (3423.7±39.7 μmol/kg) in the raw broccoli sample. It seems that the combined preheating and fermentation process enhances the release and accessibility of glucoraphanin for conversion over and above the inactivation of ESP by the pre-heating process. The pre-heating process coupled with microbial cell wall degrading enzymes may have enhanced the disruption of the cell compartment and release of bound glucosinolates in the matrix, that were not extractable or accessible in the raw broccoli. Some lactic acid strains produce polysaccharide degrading enzymes such as cellulases and pectinases capable of degrading the cell wall structure and enhance the release of wall bound components.

In contrast, chemical acidification of preheated broccoli puree by GDL resulted in a significantly lower (p<0.05) content of sulforaphane compared to pre-heated and preheat-fermented samples (Table 6). The sulforaphane content of the GDL acidified samples were 2169.4±176.0 μmol/kg DW, which is 40% lower than the preheated broccoli sample (3536.01136.9 μmol/kg DW) (P<0.05). It appears that the fast reduction to pH 4.04 during acidification may have reduced the conversion of glucoraphanin into sulforaphane in the GDL samples. It is well known that the conversion of glucosinolates is highly dependent on pH and acidic pH favours conversion into nitriles (Latte et al., 2011).

In the case of the pre-heated fermented samples, the acidification occurs gradually over a period of >15 hr enabling the conversion of glucoraphanin mainly to sulforaphane since the activity of ESP is expected to be significantly reduced after preheating at 65° C. for 3 min.

### Changes of Sulforaphane Content During Storage

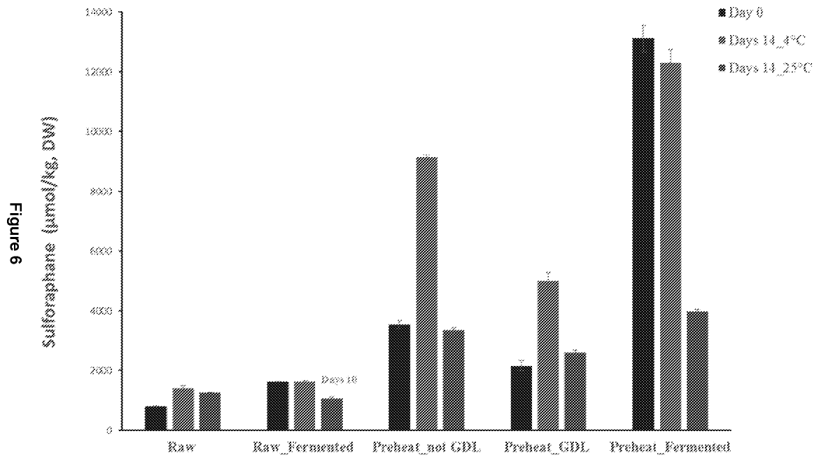

The concentration of sulforaphane of all the samples declined after 14 days storage at 25° C. (see Table 6 and FIG. 6). Interestingly, an increase in sulforaphane content was observed in all samples except the fermented samples during 14 days storage at 4° C. The sulforaphane content of the raw puree almost doubled during storage at 4° C. Similarly, the sulforaphane content of the pre-heated samples increased by ˜2.6 times whereas the sulforaphane content of the preheated GDL samples increased by ˜2.3 times, which suggests continuous release of glucoraphanin from the matrix during storage allowing further conversion to sulforaphane and increase in concentration counteracting the consequence of sulforaphane degradation during storage. With respect to the preheated-fermented samples, reduction in sulforaphane content was observed during storage at both temperatures. All the accessible glucoraphanin may have been converted to sulforaphane during fermentation so much so that no further conversion occurred during storage but rather degradation albeit to a different extend depending on the temperature. As such, only a slight decline (˜6%) was observed during storage at 4° C. whereas the decline during storage at 25° C. was ˜70%.

TABLE 6
Sulforaphane yield (μmol/Kg DW) of broccoli before and after processing.

| | Sulforaphane (μmol/kg, DW) | | | | |
|---|---|---|---|---|---|
| | | Raw- | Preheatnot | | Preheat- |
| | Raw | Fermented | GDL | Preheat GDL | Fermented |
| Day 0 | 806.2 ± 7.0 | 1617.4 ± 10.2 | 3536.0 ± 136.9 | 2169.4 ± 176.0 | 13121.3 ± 440.8 |
| Days | 1409.8 ± 82.7 | 1627.7 ± 17.5 | 9149.4 ± 63.6 | 4994.8 ± 291.2 | 12301.3 ± 443.5 |
| 14_4° C. | | | | | |
| Days | 1268.2 ± 0.1 | 1065.8 ± 49.8 | 3338.2 ± 93.9 | 2593.1 ± 97.7 | 3974.2 ± 71.2 |
| 14_25° C. | | | | | |

DW: dry weight,
GDL: acidified using glucono-delta-lactone.
Preheating was conducted at 65° C. in pack for 3 minutes.

This study showed that pre-heating coupled with lactic acid bacteria fermentation substantially enhances the sulforaphane content of broccoli based products. In-pack pre-heating treatment of broccoli florets at 65° C. for 3 min followed by maceration and fermentation resulted in as much as ˜16 times higher yield of sulforaphane compared to raw broccoli puree. Preheating under this condition increased the sulforaphane yield in broccoli puree from 806 mol/KgDW (dry weight) in the untreated broccoli to 3536 mol/KgDW, indicating that the treatment substantially inhibits ESP while maintaining sufficient myrosinase activity for the conversion of glucoraphanin into sulforaphane. The best preheating condition during direct water blanching was 1 min at 60° C. and resulted in sulforaphane yield of 2833 mol/KgDW. The lower yield during direct blanching can be attributed to leaching of the water-soluble glucoraphanin into the blanching media. Preheating of broccoli florets in-pack (65° C./3 min) combined with lactic acid bacteria fermentation further enhanced the sulforaphane content to 13121 mol/KgDW, which is ˜16 times increase compared to raw broccoli. Chemical acidification of in-pack preheated (65° C., 3 min) combined with acidification of the broccoli puree by glucono-delta-lactone resulted in sulforaphane yield of 2169 mol/KgDW, which is lower than pre-heating alone. The sulforaphane content of the preheated-fermented puree remained stable (˜94% retention) during two weeks storage at 4° C.

### Example 12—Effect of Lactic Acid Bacteria Fermentation on Polyphenolic Profile of Broccoli

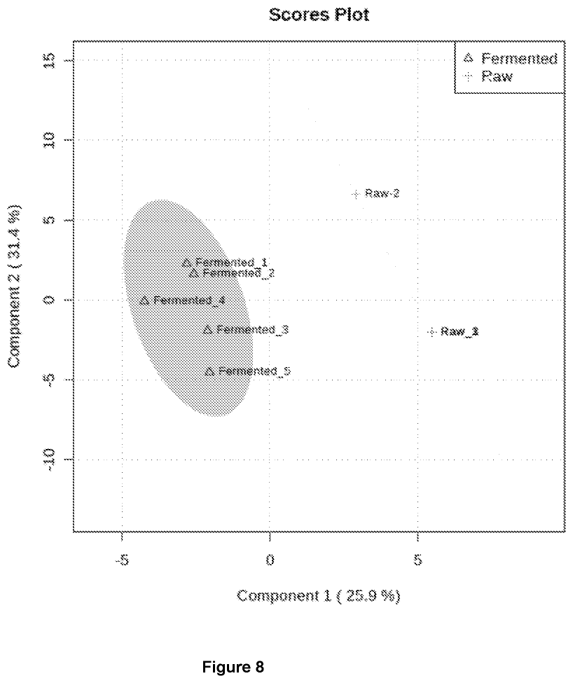

In order to determine the effects of fermentation on the polyphenolic metabolites of broccoli samples, targeted liquid chromatography-mass spectrometry (LC-MS) based metabolomic analysis of the raw and fermented broccoli puree samples was conducted. The resulting multivariate data was analysed using Metaboanalyst software (Metaboanalyst 3.0, Xia and Wishart, 2016). Fermentation resulted in a significant change in the metabolite profile of the broccoli samples. The partial least square discriminant analysis (PLS-DA) of the data shows a clear distinction between the polyphenolic profile of the fermented and the non-fermented samples (FIG. 8).

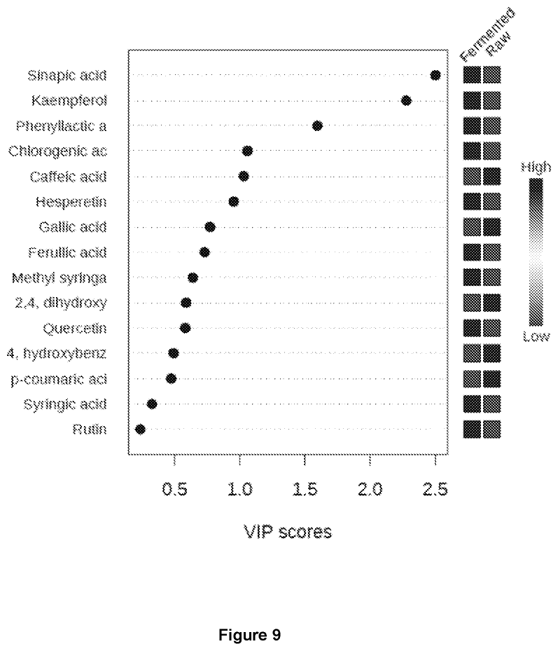

The top 15 metabolites that were identified to be responsible for the differences between the two groups are shown in FIG. 9. They are phenolic acids and phenolic aglycones, with higher bioactivity and bioavailability compared to their phenolic acid ester and phenolic glycoside precursors. The concentrations of most of these metabolites showed substantial increase following fermentation indicating the beneficial effect of fermentation on the polyphenol profile of broccoli puree. The fold changes for some of the metabolites are shown in Table 7.

A substantial increase in sinapic acid and kaempferol, 24 fold and 16 fold respectively was observed following fermentation. Similarly, fermentation induced an 8 fold increase in chlorogenic acid and phenyllactic acid. The concentrations of hesperetin, quercetin, methyl syringate and syringic acid also increased substantially after fermentation. The increase in the concentration of aglycones such as kaempferol, hesperetin and quercetin can be attributed to conversion of their glycoside precursors by the activity of microbial glycosidases. The increase in the concentration of phenolic acids such as sinapic acid could be due to the conversion of phenolic acid esters in broccoli by the activity of microbial esterases. Some decrease in caffeic acid and gallic was observed following fermentation. The activity of microbial decarboxylases convert caffeic acid into the corresponding vinyl catechol and gallic acid into pyrgallol, which may be responsible for the decrease in their concentration (Filanino et al., 2015; Guzman-Lopez et al., 2009).

TABLE 7
Fold changes in the top 13 polyphenols responsible for differences
between fermented and non-fermented broccoli puree.

| | | Fold change | Log₂ |
|---|---|---|---|
| | Compounds | (FC) | (FC) |
| 1 | Sinapic acid | 24.1 | 4.6 |
| 2 | Kaempferol | 16.1 | 4.0 |
| 3 | Chlorogenic acid | 8.3 | 3.1 |
| 4 | Phenyllactic acid | 7.9 | 3 |
| 5 | Hespertin | 3.7 | 1.9 |
| 6 | Methyl syringate | 3.3 | 1.7 |
| 7 | Syringic acid | 3.3 | 1.7 |
| 8 | Caffeic acid | 0.32 | −1.6 |
| 9 | Ferullic acid | 2.7 | 1.4 |
| 10 | 4,hydroxybenzoic acid | 0.4 | −1.4 |
| 11 | Quercetin | 2.6 | 1.3 |
| 12 | Rutin | 2.5 | 1.3 |
| 13 | Gallic acid | 0.5 | −1.1 |

### Example 13—Identification of Metabolites Produced by Lactic Acid Bacteria Fermentation of Broccoli by Targeted and Untargeted LC MS Analyses of Samples

The fermented and non-fermented broccoli puree samples were frozen and freeze dried. The samples (100 mg freeze dried powder each) were extracted using 1 ml of ice-cold methanol and Milli-Q water (50:50, v:v), which comprised 100 mg/ml of caffeine as an internal standard. The samples were then vortexed for 2 minutes prior to being sonicated (40 Hz) for 30 minutes. Samples were then centrifuged at 20,000 rpm at 4° C. for 30 minutes, and the supernatant transferred to clean silanised LC-MS vials. Samples were analyzed by injecting 1.4 μl into an Agilent 6410 LC-QQQ HPLC (Agilent Technologies, Santa Clara, Calif., USA). The analyses were performed using a reversed-phase Agilent Zorbax Eclipse Plus C18, Rapid Resolution HD, 2.1×50 mm, 1.8 μm (Agilent Technologies, Santa Clara, Calif., USA), with a column temperature of 30° C. and a flow rate of 0.3 ml/min. The mobile phase was operated isocratically for 1 min 95:5 (A:B) then switched to 1:99 (A:B) for a further 12 min before returning back to 95:5 (A:B) for an additional 2 min; providing a total run time of 15 min. Mobile phase ‘A’ consisted of 100% H₂O and 0.1% formic acid, and mobile phase ‘13’ contained 75% acetonitrile, 25% isopropanol and 0.1% formic acid. The MS was collecting data in the mass range 50-1000 m/z. Qualitative identification of the compounds was performed according to the Metabolomics Standard Initiative (MSI) Chemical Analysis Workgroup using several online LC-MS metabolite databases, including Massbank and METLIN. Overall, the instrumental conditions were similar for both positive electrospray (+ESI) and negative electrospray (−ESI) modes. Scan time was 500, the source temperature was maintained at 350° C., the gas flow was 12 L/min and the nebuliser pressure was 35 psi.

For the identification of compounds in the untargeted analysis, the criteria was set at >90% match rate. Where the match rate dropped to between 70-89%, the compounds are identified with brackets (for example, if a compound was between 70-89% they are annotated as “<name>”). Any matches below 70% were removed. In total, there was ca. 1000-1500 features to identify; many were poorly matched (and removed) or were less than 10×S/N ratio from the baseline. As such, the compounds/peaks used were actual peaks and the IDs are fairly strong (i.e. >70%).

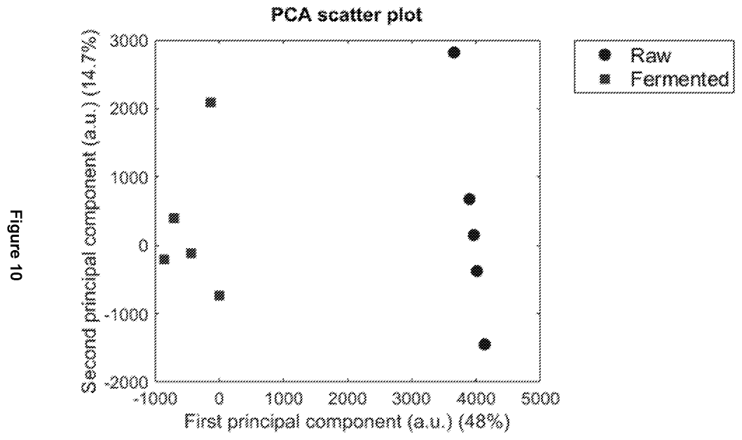

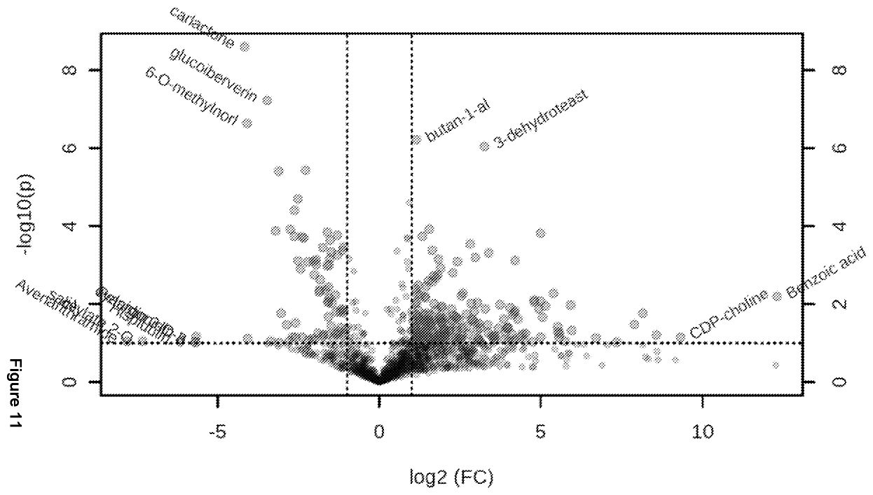

Untargeted LC-MS metabolomics study showed a 2 to 360 fold increase in certain polyphenolic glycosides including anthocyanin glycosides, phenolic acid glycosides, phenolic acids, a 5 to 60 fold increase in some glucosinolates with glucoraphanin increasing 27 fold and about a 3 to 4 fold increase in indol-3carbinol and ascorbigen. Results are summarised in Table 8 and are shown in FIG. 10 and in a volcano plot in FIG. 11. The top 50 metabolites that increased after fermentation include several polyphenol glycosides and glucosinolates indicating that the process enhances their extractability and bioaccessibility.

TABLE 8
Fold changes in different metabolites between fermented and non-
fermented broccoli puree based on untargeted LC-MS analysis.

| Metabolite | FC | log2(FC) | raw.pval | (−LOG10(p)) |
|---|---|---|---|---|
| Benzoic acid | 4670.1 | 12.189 | 5.50E−08 | 7.2593 |
| Cyanidin 3-O-rutinoside | 361.03 | 8.496 | 0.011951 | 1.9226 |
| Cyanidin 3-O-6″-p-coumaroyl-glucoside | 271.87 | 8.0868 | 0.011465 | 1.9406 |
| molybdopterin | 149.51 | 7.2241 | 0.00915 | 2.0386 |
| 5-methylthiopentylglucosinolate | 59.335 | 5.8908 | 0.005835 | 2.234 |
| 5-methylthioribulose 1-phosphate | 46.001 | 5.5236 | 0.000334 | 3.4757 |
| Ellagic acid arabinoside | 42.956 | 5.4248 | 0.002845 | 2.546 |
| thiamine phosphate | 42.436 | 5.4072 | 0.005123 | 2.2905 |
| 2-carboxy-D-arabinitol 1-phosphate | 41.06 | 5.3597 | 0.013093 | 1.883 |
| N-acetyl-D-glucosamine 1,6-bisphosphate | 40.636 | 5.3447 | 0.001824 | 2.739 |
| S-norreticuline | 32.883 | 5.0393 | 0.000362 | 3.4412 |
| 5-formamido-1-5-phospho-D-ribosyl- | 30.585 | 4.9348 | 8.28E−06 | 5.0817 |
| imidazole-4-carboxamide | | | | |
| 4-methylumbelliferone 6′-O- | 30.436 | 4.9277 | 0.001329 | 2.8765 |
| malonylglucoside | | | | |
| Hydroxytyrosol 4-O-glucoside | 28.971 | 4.8565 | 0.001319 | 2.8798 |
| glucoraphanin | 27.475 | 4.7801 | 0.014685 | 1.8331 |
| glucobrassicin | 26.746 | 4.7413 | 0.00441 | 2.3556 |
| 5-hydroxy-CMP | 25.864 | 4.6929 | 0.004277 | 2.3689 |
| 4alpha-formyl,4beta,14alpha-dimethyl- | 18.8 | 4.2326 | 0.003497 | 2.4563 |
| 9beta,19-cyclo-5alpha-ergost-24241-en- | | | | |
| 3beta-ol | | | | |
| indole-3-acetyl-phenylalanine | 17.44 | 4.1243 | 2.37E−06 | 5.6245 |
| N-hydroxypentahomomethionine | 16.92 | 4.0807 | 0.000559 | 3.2529 |
| Cyanidin 3-O-arabinoside | 16.098 | 4.0088 | 0.000413 | 3.3837 |
| tetrahydrobiopterin | 15.412 | 3.946 | 0.015746 | 1.8028 |
| orotidine 5′-phosphate | 14.737 | 3.8813 | 0.001699 | 2.7699 |
| 2-2′-methylthiopentylmaleate | 14.621 | 3.87 | 0.005417 | 2.2662 |
| S-adenosyl 3-methylthiopropylamine | 14.564 | 3.8644 | 0.00177 | 2.752 |
| 4-methylthiobutyl glucosinolate | 14.183 | 3.8261 | 0.011178 | 1.9516 |
| salicylate | 13.59 | 3.7644 | 0.000221 | 3.6556 |
| N-hydroxyhomomethionine | 12.902 | 3.6896 | 0.004311 | 2.3654 |
| 4′-phosphopantetheine | 11.775 | 3.5576 | 0.003073 | 2.5124 |
| 5-phospho-beta-D-ribosylamine | 10.643 | 3.4119 | 0.003185 | 2.497 |
| D-erythro-imidazole-glycerol-phosphate | 10.288 | 3.3629 | 0.019147 | 1.7179 |
| a reduced flavodoxin | 10.108 | 3.3374 | 0.005373 | 2.2698 |
| Cyanidin 3-O-6″-dioxalyl-glucoside | 9.9207 | 3.3104 | 0.000299 | 3.5242 |
| 8-oxo-GMP | 9.8883 | 3.3057 | 0.008524 | 2.0694 |
| 3-dehydroteasterone | 8.985 | 3.1675 | 8.33E−09 | 8.0793 |
| indolylmethylisothiocyanate | 7.7651 | 2.957 | 0.018337 | 1.7367 |
| choline | 7.7212 | 2.9488 | 0.023412 | 1.6306 |
| carbamoyl phosphate | 7.7098 | 2.9467 | 0.009139 | 2.0391 |
| homogentisate | 7.6608 | 2.9375 | 0.00153 | 2.8153 |
| S-adenosyl-L-methionine | 7.3817 | 2.8839 | 2.85E−05 | 4.5445 |
| oxaloacetate | 7.3494 | 2.8776 | 0.000538 | 3.2694 |
| urate | 7.2329 | 2.8546 | 0.000803 | 3.0951 |
| coniferaldehyde glucoside | 7.1826 | 2.8445 | 0.016973 | 1.7702 |
| pyridoxal 5′-phosphate | 7.0734 | 2.8224 | 0.021829 | 1.661 |
| dTMP | 6.9501 | 2.797 | 0.018743 | 1.7272 |
| 2-oxoglutarate | 6.8749 | 2.7813 | 0.00019 | 3.7216 |
| coniferaldehyde | 6.6643 | 2.7365 | 1.46E−05 | 4.8345 |
| Petunidin 3-O-rhamnoside | 6.0484 | 2.5965 | 0.002487 | 2.6043 |
| 6-phospho D-glucono-1,5-lactone | 5.8171 | 2.5403 | 0.019384 | 1.7126 |
| dTDP | 5.6526 | 2.4989 | 0.000837 | 3.0774 |
| propane-1,3-diamine | 5.5793 | 2.4801 | 0.001873 | 2.7275 |
| benzoate | 5.4402 | 2.4437 | 0.005218 | 2.2825 |
| xi-progoitrin | 5.091 | 2.3479 | 0.000107 | 3.9715 |
| 2-phospho-D-glycerate | 5.0613 | 2.3395 | 0.001146 | 2.941 |
| R-4′-phosphopantothenoyl-L-cysteine | 4.8855 | 2.2885 | 0.01357 | 1.8674 |
| L-arogenate | 4.782 | 2.2576 | 0.018843 | 1.7248 |
| L-phenylalanine | 4.5585 | 2.1886 | 0.000213 | 3.671 |
| Phenol | 4.4651 | 2.1587 | 0.002537 | 2.5956 |
| Gardenin B | 4.3888 | 2.1338 | 0.012372 | 1.9076 |
| glucomalcommin | 4.1855 | 2.0654 | 0.014526 | 1.8378 |
| Sulfachloropyridazine | 4.1627 | 2.0575 | 0.013676 | 1.864 |
| 4-methyl-2-oxopentanoate | 3.906 | 1.9657 | 0.004372 | 2.3593 |
| ascorbigen | 3.7819 | 1.9191 | 0.017398 | 1.7595 |
| 2-naphthol | 3.6366 | 1.8626 | 0.01404 | 1.8526 |
| Medioresinol | 3.6131 | 1.8532 | 0.007717 | 2.1125 |
| E-2-pentenol | 3.5473 | 1.8267 | 0.012466 | 1.9043 |
| N-feruloyltyramine | 3.3648 | 1.7505 | 0.004573 | 2.3399 |
| 2-methyl-6-phytyl-1,4-benzoquinol | 3.3442 | 1.7417 | 0.000245 | 3.6101 |
| pyridoxal | 3.0278 | 1.5983 | 0.00016 | 3.7954 |
| 1D-myo-inositol 1-monophosphate | 2.784 | 1.4771 | 0.005472 | 2.2618 |
| N-monomethylethanolamine | 2.7546 | 1.4618 | 1.55E−05 | 4.8092 |
| 3,4-Dicaffeoylquinic acid | 2.7368 | 1.4525 | 0.012553 | 1.9013 |
| Cirsilineol | 2.6151 | 1.3868 | 0.001515 | 2.8197 |
| S-methylmalonate-semialdehyde | 2.5477 | 1.3492 | 0.012237 | 1.9123 |
| benzaldehyde | 2.5268 | 1.3373 | 0.01558 | 1.8074 |
| Unidentified metabolite No. 1 | 2.3799 | 1.2509 | 7.84E−05 | 4.1056 |
| Isorhamnetin | 2.2605 | 1.1766 | 0.001828 | 2.738 |
| AMP | 2.1939 | 1.1335 | 0.002464 | 2.6083 |
| 2-Hydroxybenzoic acid | 2.1338 | 1.0935 | 0.006072 | 2.2167 |
| butan-1-al | 2.0853 | 1.0602 | 3.16E−07 | 6.5005 |
| 7-Hydroxymatairesinol | 2.0626 | 1.0445 | 0.008034 | 2.095 |
| Dimethylmatairesinol | 0.43475 | −1.2018 | 0.000284 | 3.5464 |
| trans-zeatin | 0.39207 | −1.3508 | 0.008484 | 2.0714 |
| Unidentified metabolite No. 2 | 0.38059 | −1.3937 | 0.000721 | 3.1421 |
| coniferyl alcohol | 0.37824 | −1.4026 | 0.011806 | 1.9279 |
| papaverine | 0.36651 | −1.4481 | 0.012288 | 1.9105 |
| 2,5-diamino-6-5-phospho-D- | 0.3594 | −1.4763 | 0.020453 | 1.6893 |
| ribosylaminopyrimidin-43H-one | | | | |
| S-4-hydroxymandelonitrile | 0.32867 | −1.6053 | 0.00375 | 2.426 |
| 22alpha-hydroxy-campest-4-en-3-one | 0.32674 | −1.6138 | 0.004969 | 2.3037 |
| 3-cyano-L-alanine | 0.32471 | −1.6228 | 0.013212 | 1.879 |
| Ellagic acid glucoside | 0.32466 | −1.623 | 0.022951 | 1.6392 |
| 2-naphthol 6′-O-malonylglucoside | 0.30641 | −1.7064 | 0.000709 | 3.1492 |
| pelargonidin | 0.30629 | −1.707 | 0.010379 | 1.9838 |
| 2S-naringenin | 0.30353 | −1.7201 | 0.019827 | 1.7027 |
| 8-methylthiooctyl-thiohydroximate | 0.28257 | −1.8233 | 0.002811 | 2.5512 |
| Stigmastanol ferulate | 0.28168 | −1.8279 | 0.017703 | 1.752 |
| Pinosylvin | 0.26912 | −1.8937 | 0.01535 | 1.8139 |
| germacra-110,4,1113-trien-12-ol | 0.23506 | −2.0889 | 0.022511 | 1.6476 |
| indole-3-acetyl-glutamine | 0.20278 | −2.302 | 0.006425 | 2.1921 |
| 2-7′-methylthioheptylmalate | 0.19682 | −2.3451 | 0.001077 | 2.968 |
| p-coumaroyltriacetic acid lactone | 0.18436 | −2.4394 | 0.0122 | 1.9136 |
| 6″-O-Acetyldaidzin | 0.15801 | −2.6619 | 0.008935 | 2.0489 |
| indole-3-acetyl-glutamate | 0.15472 | −2.6922 | 0.003623 | 2.441 |
| Isorhamnetin 3-O-glucoside 7-O- | 0.15357 | −2.703 | 0.002647 | 2.5773 |
| rhamnoside | | | | |
| olivetol | 0.13094 | −2.933 | 0.005902 | 2.229 |
| N-hydroxy-L-phenylalanine | 0.1141 | −3.1316 | 0.000812 | 3.0905 |
| R-pantothenate | 0.10725 | −3.221 | 1.36E−05 | 4.8679 |
| glucoiberverin | 0.087316 | −3.5176 | 0.00014 | 3.8538 |
| 6-O-methylnorlaudanosoline | 0.055734 | −4.1653 | 6.96E−05 | 4.1575 |
| carlactone | 0.052932 | −4.2397 | 2.93E−05 | 4.5332 |
| E,E-geranyllinalool | 0.018254 | −5.7757 | 0.004044 | 2.3932 |
| UDP-alpha-D-xylose | 13.367 | 3.7407 | 0.0235 | 1.6289 |
| Z-1-glutathione-S-yl-2-phenyl- | 19.906 | 4.3151 | 0.026163 | 1.5823 |
| acetohydroximate | | | | |
| Apigenin 7-O-6″-malonyl-apiosyl- | 0.38092 | −1.3925 | 0.02641 | 1.5782 |
| glucoside | | | | |
| 4alpha-formyl-stigmasta-7,24241-dien- | 58.691 | 5.8751 | 0.026582 | 1.5754 |
| 3beta-ol | | | | |
| soyasapogenol B | 0.35836 | −1.4805 | 0.027448 | 1.5615 |
| dihydroconiferyl alcohol glucoside | 5.6248 | 2.4918 | 0.027644 | 1.5584 |
| 3-deoxy-alpha-D-manno-octulosonate | 6.6012 | 2.7227 | 0.027652 | 1.5583 |
| Anhydro-secoisolariciresinol | 2.3975 | 1.2616 | 0.027928 | 1.554 |
| 3-isopropyl-7-methylthio-2-oxoheptanoate | 0.30287 | −1.7232 | 0.028072 | 1.5517 |
| Kaempferide | 0.15749 | −2.6666 | 0.0281 | 1.5513 |
| 2-aminoprop-2-enoate | 2.0003 | 1.0002 | 0.029166 | 1.5351 |
| isoliquiritigenin | 2.8505 | 1.5112 | 0.029212 | 1.5344 |
| m-Coumaric acid | 2.187 | 1.129 | 0.029331 | 1.5327 |
| indole-5,6-quinone | 2.6937 | 1.4296 | 0.02956 | 1.5293 |
| 2-4′-methylthiobutylmalate | 0.43617 | −1.197 | 0.030711 | 1.5127 |
| 7-methylthioheptyl glucosinolate | 0.42422 | −1.2371 | 0.030739 | 1.5123 |
| camalexin | 0.27584 | −1.8581 | 0.030778 | 1.5118 |
| 3-Methoxynobiletin | 8.9717 | 3.1654 | 0.031528 | 1.5013 |
| 8-methylsulfinyloctyl glucosinolate | 0.1694 | −2.5615 | 0.031733 | 1.4985 |
| ent-cassa-12,15-diene | 0.33285 | −1.587 | 0.032806 | 1.484 |
| Catechol | 4.0005 | 2.0002 | 0.033382 | 1.4765 |
| L-aspartate-semialdehyde | 2.9298 | 1.5508 | 0.033499 | 1.475 |
| 10-methylthio-2-oxodecanoate | 4.5655 | 2.1908 | 0.033543 | 1.4744 |
| indole-3-carbinonium ion | 2.7807 | 1.4754 | 0.033654 | 1.473 |
| laurate | 0.33955 | −1.5583 | 0.034205 | 1.4659 |
| malonate | 9.0975 | 3.1855 | 0.035699 | 1.4473 |
| 1-aci-nitro-8-methylsulfanyloctane | 8.8356 | 3.1433 | 0.035865 | 1.4453 |
| 2-hydroxy-5-methylthio-3-oxopent-1-enyl | 13.56 | 3.7612 | 0.036727 | 1.435 |
| 1-phosphate | | | | |
| glyoxylate | 16.835 | 4.0734 | 0.037951 | 1.4208 |
| Feruloyl tartaric acid | 5.5489 | 2.4722 | 0.038578 | 1.4137 |
| 3beta-hydroxyparthenolide | 8.1691 | 3.0302 | 0.038749 | 1.4117 |
| 22R,23R-22,23-dihydroxycampesterol | 2.0564 | 1.0401 | 0.039305 | 1.4056 |
| Gallic acid 4-O-glucoside | 2.515 | 1.3306 | 0.039605 | 1.4023 |
| E-phenylacetaldoxime | 2.1608 | 1.1116 | 0.040641 | 1.391 |
| 18-hydroxystearate | 0.14519 | −2.784 | 0.042027 | 1.3765 |
| 5′-phosphoribosyl-4-N- | 0.4281 | −1.224 | 0.042243 | 1.3742 |
| succinocarboxamide-5-aminoimidazole | | | | |
| 3-Feruloylquinic acid | 3.3496 | 1.744 | 0.042655 | 1.37 |
| 2-carboxy-L-threo-pentonate | 2.0447 | 1.0319 | 0.043 | 1.3665 |
| trans-zeatin riboside | 0.40453 | −1.3057 | 0.044527 | 1.3514 |
| 4-fumaryl-acetoacetate | 5.0298 | 2.3305 | 0.044744 | 1.3493 |
| 2-cis-abscisate | 76.81 | 6.2632 | 0.044918 | 1.3476 |
| 4-Hydroxycoumarin | 0.48212 | −1.0525 | 0.045785 | 1.3393 |
| Biochanin A | 2.1017 | 1.0716 | 0.046533 | 1.3322 |
| S-2,3,4,5-tetrahydrodipicolinate | 4.1401 | 2.0497 | 0.046976 | 1.3281 |
| 26,27-dehydrozymosterol | 14.846 | 3.892 | 0.047042 | 1.3275 |
| N-methylethanolamine phosphate | 10.038 | 3.3273 | 0.047416 | 1.3241 |
| Kaempferol 3-O-2″-rhamnosyl-galactoside | 2.7008 | 1.4334 | 0.048201 | 1.3169 |
| 7-O-rhamnoside | | | | |
| pheophorbide a | 6.3398 | 2.6644 | 0.049365 | 1.3066 |
| Chrysoeriol 7-O-6″-malonyl-glucoside | 4.8949 | 2.2913 | 0.049727 | 1.3034 |
| allantoate | 10.972 | 3.4557 | 0.050008 | 1.301 |
| Ligstroside-aglycone | 12.072 | 3.5936 | 0.052404 | 1.2806 |
| cycloeucalenone | 3.4926 | 1.8043 | 0.052645 | 1.2786 |
| Unidentified metabolite No. 3 | 3.5807 | 1.8403 | 0.053727 | 1.2698 |
| laricitrin | 0.42811 | −1.224 | 0.05399 | 1.2677 |
| Sulfadimethoxine | 11.488 | 3.5221 | 0.05455 | 1.2632 |
| 3,4-Diferuloylquinic acid | 5.2839 | 2.4016 | 0.054583 | 1.2629 |
| glucotropeolin | 0.47952 | −1.0603 | 0.054637 | 1.2625 |
| 5,6-dihydroxyindole-2-carboxylate | 5.2663 | 2.3968 | 0.055218 | 1.2579 |
| S-laudanine | 2.8697 | 1.5209 | 0.055638 | 1.2546 |
| L-nicotianamine | 0.39854 | −1.3272 | 0.057257 | 1.2422 |
| 5-methylthiopentyl-thiohydroximate | 0.30202 | −1.7273 | 0.057551 | 1.2399 |
| aldehydo-D-galacturonate | 2.6643 | 1.4138 | 0.05785 | 1.2377 |
| R-mevalonate 5-phosphate | 0.34888 | −1.5192 | 0.058188 | 1.2352 |
| 6-Hydroxyluteolin 7-O-rhamnoside | 2.142 | 1.099 | 0.05845 | 1.2332 |
| L-aspartate | 3.5705 | 1.8361 | 0.061441 | 1.2115 |
| --Epicatechin 3-O-gallate | 2.4481 | 1.2916 | 0.063269 | 1.1988 |
| glycine | 0.23586 | −2.084 | 0.065585 | 1.1832 |
| Episesaminol | 2.4077 | 1.2677 | 0.065876 | 1.1813 |
| 6alpha-hydroxy-castasterone | 3.7782 | 1.9177 | 0.068376 | 1.1651 |
| alpha-D-galacturonate 1-phosphate | 11.846 | 3.5664 | 0.070966 | 1.149 |
| R-2,3-dihydroxy-3-methylpentanoate | 2.995 | 1.5825 | 0.071057 | 1.1484 |
| cyanidin-3-O-beta-D-glucoside | 2.0686 | 1.0487 | 0.07128 | 1.147 |
| D-erythrose 4-phosphate | 3.7463 | 1.9054 | 0.07247 | 1.1398 |
| CDP-choline | 617.84 | 9.2711 | 0.073728 | 1.1324 |
| adenine | 2.0623 | 1.0442 | 0.074004 | 1.1307 |
| raphanusamate | 5.5593 | 2.4749 | 0.074387 | 1.1285 |
| 3-Methoxysinensetin | 2.4046 | 1.2658 | 0.075102 | 1.1243 |
| betaine aldehyde | 3.5234 | 1.817 | 0.075291 | 1.1233 |
| E-7-methylthioheptanaldoxime | 2.2972 | 1.1999 | 0.076906 | 1.114 |
| 6-methylthiohexyl-thiohydroximate | 5.5473 | 2.4718 | 0.077579 | 1.1103 |
| 6″-O-Malonylglycitin | 0.16741 | −2.5786 | 0.080677 | 1.0933 |
| monodehydroascorbate radical | 2.0677 | 1.048 | 0.081844 | 1.087 |
| anthranilate | 3.0289 | 1.5988 | 0.082088 | 1.0857 |
| Hydroxycaffeic acid | 0.43234 | −1.2098 | 0.082209 | 1.0851 |
| Myricetin 3-O-arabinoside | 2.3978 | 1.2617 | 0.086518 | 1.0629 |
| cis-aconitate | 0.18331 | −2.4477 | 0.088998 | 1.0506 |
| 5-phospho-alpha-D-ribose 1-diphosphate | 0.47829 | −1.064 | 0.089065 | 1.0503 |
| Malvidin 3-O-glucoside | 0.48171 | −1.0538 | 0.089472 | 1.0483 |
| N6-delta2-isopentenyl-adenosine 5′- | 44.241 | 5.4673 | 0.092566 | 1.0335 |
| monophosphate | | | | |
| Quercetin 3-O-6″-acetyl-galactoside 7-O- | 2.9914 | 1.5808 | 0.093824 | 1.0277 |
| rhamnoside | | | | |
| cholesterol | 2.816 | 1.4936 | 0.095163 | 1.0215 |
| 9-methylthiononyl-thiohydroximate | 15.416 | 3.9464 | 0.098598 | 1.0061 |

In order to determine the effects of fermentation on the polyphenolic metabolites of broccoli samples, targeted liquid chromatography-mass spectrometry (LC-MS) based metabolomic analysis of the raw and fermented broccoli puree samples was conducted. Statistical analysis was performed without preprocessing. Fermentation resulted in a significant change in the metabolite profile of the broccoli samples.

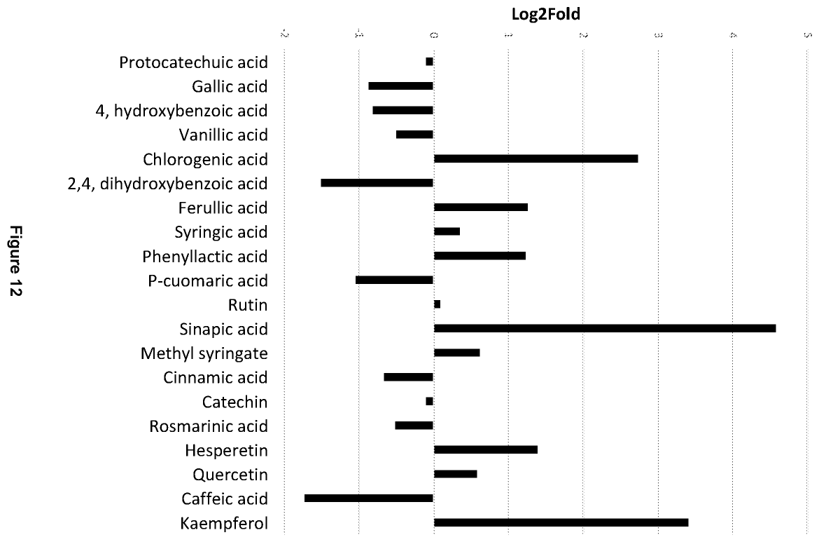

In the targeted LC-MS analysis, polyphenol standards were used for the identification and quantification of the metabolites. Increases in chlorogenic acid, ferullic acid, syringic acid, phenyllactic acid, rutin, sinapic acid, methyl syringate, hesperetin, quercetin and kaempferol were confirmed in fermented broccoli (FIG. 12). Decreases in protocatechuic acid, gallic acid, 4,hydroxybenzoic acid, vanillic acid, 2,3dihydroxybenzoic acid, p-cuomaric acid, cinnamic acid, catechin, rosmarinic acid, caffeic acid were confirmed in fermented broccoli (FIG. 12). Of note is that a 6.6 fold change in chlorogenic acid (2.4 to 15.8 μg/mg), a 23.8 fold increase is in sinapic acid (3.6 to 86.6 μg/mg), a 10.5 increase in kaempferol (12.7 to 134.6 μg/mg) and a 0.48 fold decrease in p-Coumaric acid occurred in fermented samples (FIG. 12).

### Example 14—Assessment of the Broccoli Fermentation Culture to Inhibit the Growth of Intentionally Introduced Microorganisms

A challenge study was conducted to assess the ability of the broccoli fermentation culture to inhibit the growth of intentionally introduced microorganisms which are often observed and of concern in food preparation.

### Lab Culture/Starter Culture

10 ml of 10¹⁰ cfu/mL of an inoculum comprising B1, B2, B3, B4, B5, BF1 and BF2 to achieve 10⁸ CFU/gm of sample in the ferment.

### Pathogen Cultures

E. coli isolates FSAW 1310, FSAW 1311, FSAW 1312, FSAW 1313 and FSAW 1314 were grown separately to 1-4×10⁸ cfu/mL in NB (nutrient broth) overnight at 37° C., static. The cultures were combined (1 mL of each) and the combined culture diluted to 10⁴ with MRD (maximum recovery diluent) for first two dilutions and water for last two dilutions.

Salmonella strains S. infantis 1023, S. singapore 1234, S. typhimurium 1657 (PT135), S. typhimurium 1013 (PT9) and S. virchow 1563 were grown separately to 1-4×10⁸ cfu/mL in NB overnight at 37° C., static. The cultures were combined (1 mL of each) and combined culture diluted to 10⁴ with MRD for first two dilutions and water for last two dilutions.

Listeria isolates Lm2987 (7497), Lm2965 (7475), Lm2939 (7449), Lm2994 (7537) and Lm2619 (7514) were grown separately in 10 mL BHI (brain heart infusion broth) overnight at 37° C. under agitation. All cultures were then combined (1 mL of each) and this cocktail was diluted using MRD for first two (1/10) dilutions and sterile deionised water for last two dilutions.

B. cerus spore crops were prepared from isolates B3078, B2603, 2601, 7571 and 7626.

### Method

Broccoli puree was prepared prior to preparing the inoculums, Broccoli: Sterile Tap Water 3:2 (900 g broccoli: 600 g water). Broccoli heads were rinsed in tap water, the stalks were cut off the broccoli with a sterile knife on a cutting board sanitised with 80% ethanol. Broccoli florets (900 g) were cut into small pieces. 450 g of broccoli pieces were placed into Thermomix bowl with all 600 g of the water. The translucent Thermomix cup/lid was sanitised with 80% ethanol and placed over the lid hole. The broccoli was chopped at speed 4 for 1 min. The second 450 g of broccoli pieces were added to the Thermomix bowl and chopped at speed 4 for 1 min. The contents were chopped for a further 5 min at speed 10 (max). After making sure the puree was indeed smooth enough, the Thermomix bowl was placed in the cool room to cool down the contents for 30 min. Following this, the bowl was put in the incubator and equilibrated to 30° C. Meanwhile the starter culture and pathogen culture (E. coli, B. cereus, Salmonella, Listeria monocytogenes) were prepared. 10 mL of LAB culture and 7.5 mL of the 10-4-diluted challenge microorganism cocktail (10⁴ cfu/mL culture in water) were added into the broccoli puree (10⁵ of B. cereus). Foil was held down over the large hole in the Thermomix lid prior to mixing culture. The cultures were mixed into the puree for 1 min on maximum speed. The heat setting for the Thermomix was switched off and the Thermomix was placed inside the 30° C. incubator and the fermentation started at 10:45 am. pH and temperature measurements were taken every hour up until 7 h (end of work time) after mixing the puree for 1 min speed 4.5. The pH meter was calibrated and sanitised using 80% ethanol. The temperature probe was also sanitised prior to measurements with 80% ethanol.

The growth of the challenge microorganisms was assessed by counts on growth on the selective media MRS, DRBX and NA +S of raw broccoli, before fermentation (TO) and after fermentation commenced at 4 hours (T4) and 22 hours (T22).

### Results

The yeast and mould were significantly reduced by 4 hours, and were not detected at the end of fermentation (T22). E. coli and Salmonella were never detected at the end of fermentation (T22). Listeria was detected in low numbers at the end of fermentation, with a starting inoculum just over 10³ cfu/mL. B. cereus spores were generally not affected by the fermentation, but did not germinate. The result of the challenge study indicates that the lactic acid bacteria strains that we isolated from broccoli are able to completely inactivate Salmonella and E. coli and inhibit the growth of the most acid resistant strains of Listeria. They are also able to inhibit the sporulation of B. cerus spores.

TABLE 9
Example of microbial challenge study with E. coli. E. coli
(mix of 5 E. coli strains EC1605, EC1606, EC1607, EC1608
inoculated (2.2 × 102 CFU/gm) into the macerated broccoli
(3:2 broccoli-water ratio) ferment to evaluate if the fermentation
starter (a consortia of B1, B2, B3, B4, B5, BF1, BF2) inhibits
the growth of E. coli. Experiments were repeated three times.
Fermentation was conducted at 30° C. for 22 hrs to pH below 4.0.

| Time | Lactic acid bacteria | Yeast and mould | | E. coli |
|---|---|---|---|---|
| (hrs) | (CFU/gm) | (CFU/gm) | | (CFU/gm) |
| 0 | 1.6 × 10⁸ | 2.4 × | 10³ | 1.6 × 10² |
| 4 | 1.5 × 10⁸ | 3 × | 10 | 1.2 × 10² |

| | | | |
|---|---|---|---|
| 22 | 3.6 × 10⁹ | <10 | <1 |

TABLE 10
Example of microbial challenge study with Salmonella. Salmonella
(A mix of 5 strains S. Infantis 1023, S. Singapore 1234, S. Typhimurium
1657 (PT135), S. Typhimurium 1013 (PT9), S. Virchow 1623)
inoculated (1.1 × 103) into macerated broccoli (3:2 broccoli-water ratio)
ferment to evaluate if the fermentation starter (a consortia of B1, B2, B3,
B4, B5, BF1, BF2) inhibits the growth of Salmonella. Experiments were
repeated three times. Fermentation was conducted at 30° C. for 22 hrs to
pH below 4.0.

| Time | Lactic acid bacteria | Yeast and mould | | Salmonella |
|---|---|---|---|---|
| (hrs) | (CFU/gm) | (CFU/gm) | | (CFU/gm) |
| 0 | 3.5 × 10⁸ | 1.4 × | 10³ | 6.4 × 10² |
| 4 | 4.2 × 10⁸ | 2 × | 10 | 3.3 × 10² |

| | | | |
|---|---|---|---|
| 22 | 1.4 × 10⁹ | <10 | <10 |

TABLE 11
Example of microbial challenge study with Listeria monocytogenes.
Listeria monocytogenes (A mix of 5 strains Lm2987 (7497), Lm2965
(7475), Lm2939 (7449), Lm2994 (7537), Lm2919 (7514)) inoculated
(1.9 × 103) into macerated broccoli (3:2 broccoli-water ratio) ferment to
evaluate if the fermentation starter (a consortia of B1, B2, B3, B4, B5,
BF1, BF2) inhibits the growth of acid resistant Listeria. Experiments
were repeated three times and the final Listeria count at the end of
fermentation ranged from <10 (undetected) to 1.1 × 10² CFU/gm.
Fermentation was conducted at 30° C. for 22 hrs to pH below 4.0.

| Time | Lactic acid bacteria | Yeast and mould | Listeria |
|---|---|---|---|
| (hrs) | (CFU/gm) | (CFU/gm) | (CFU/gm) |
| 0 | 5.6 × 10⁸ | 5.2 × 10⁴ | 2.1 × 10³ |
| 4 | 4.1 × 10⁸ | 3.6 × 10³ | 2.8 × 10³ |
| 22 | 5.1 × 10⁹ | <10 | 2 × 10 |

TABLE 12
Example of microbial challenge study with Bacillus cereus. Bacillus
cereus (A mix of 5 strains B3078, B2603, B2601, B7571, B7626)
inoculated (1.9 × 103) into macerated broccoli (3:2 broccoli-water ratio)
ferment to evaluate if the fermentation starter (a consortia of B1, B2, B3,
B4, B5, BF1, BF2) inhibits the growth of acid resistant Listeria.
Experiments were repeated three times. Fermentation was conducted at
30° C. for 22 hrs to pH below 4.0.

| Time | Lactic acid bacteria | Yeast and mould | Listeria |
|---|---|---|---|
| (hrs) | (CFU/gm) | (CFU/gm) | (CFU/gm) |
| 0 | 2.4 × 10⁸ | 1.2 × 10³ | 3.1 × 10³ |
| 4 | 3.3 × 10⁸ | 9.5 × 10 | 2.3 × 10³ |
| 22 | 1.9 × 10⁹ | <10 | 1.7 × 10³ |

### Example 15—Pulse Filed Gel Electrophoreses of Leuconostoc mesenteroides Isolates

Leuconostoc mesenteroides from vegetables was assessed with SmaI and NotI restriction enzyme digestion with pulse filed gel electrophoreses as described in Chat and Dalmasso (2015) with modification.

### Methods:

### Day 1

Assessed isolates were inoculated into 10 mL MRS broth and incubated overnight at 30° C. in incubator (16 h).

### Day 2

Isolates were centrifuge at 3500 g for 10 min and the supernatant discarded. The pellet was mixed and washed with 5 mL deionised water and centrifuged at 3500 g for 10 min and the supernatant discarded. The pellet was mixed with 5 mL TES (1 mM EDTA, 10 mM Tris-HCl, 0.5 M saccharose) and vortexed. Next the samples were centrifuged at 3500 g for 15 min and the supernatant discarded. 700 μL of Lysis solution (TE buffer (1 mM EDTA, 10 mM Tris-HCl, pH 8.0, sterilise as normal) with lysozyme at 10 mg/mL) was added to the pellet and mixed and incubated at 56° C. for 2 h to lyse bacteria. Next, 700 μL of agarose (1% SeaChem Gold agarose with 50 μL EDTA/100 mL) was added to the cell mixture, mix and dispensed into plug moulds and 2 mL of deproteinisation (660 μL of proteinase K buffer, 11 μL proteinase K) solution added all plugs for one sample placed in the tube and incubated at 55° C. overnight.

### Day 3

Next the plugs were heated in 100 mL of sterile deionised water at 55° C., the deproteinisation solution was removed and the plugs transferred to 15 mL centrifuge tubes, washed with 4 mL of sterile deionised water and heated to 55° C. for 10 min at room temperature followed by washing four times with 4 mL TE buffer for 10 min at room temperature.

### Restriction Digests

2 mm slice off plug was placed in an eppendorf tube with 100 μL 1× restriction buffer, incubated for 20 min at room temperature, restriction buffer was removed and replaced with 40-100 μL of SmaI (20 U) or NotI in restriction buffer and incubated for 4 h at the optimum temperature (25° C.).

### Day 4

### Separation of Restriction Fragments

1 mL 0.5×TBE buffer to each tube and allowed to sit for at least 15 min to stop reaction and the bacteriophage DNA ladder (New England Biolab) was incubated in TBE buffer. The buffer was removed and the slices loaded onto comb, with the ladder in every five lanes. 1.0% ultra-pure DNA grade agarose (pulsed field certified agarose) was prepared in 0.5×TBE running buffer.

### Electrophoresis Conditions

Buffer maintained at 14° C. (model 1000 Mini-chiller, BioRad). BioRad “Chef Mapper™”, select Two State Program (not Auto Algorithm). Pulse time ramped linearly (press enter when “a” appears) from 2 to 25 s. Gradient 6 V/cm (voltage), Included angle 120°, Running time of 24 h.

### Day 5

Gels stained ˜30 min in GelRed, destained, visualised

### Results

The restriction fingerprint for BF1 was district but similar to Leuconostoc mesenteroides isolated from carrot (FIG. 13). The restriction fingerprint for BF2 was district from all Leuconostoc mesenteroides strains assessed (FIG. 13).

### Example 16—Variant Analysis of Leuconostoc mesenteroides and Lactobacillus plantarum Isolates

For the SNP analysis of the Lactobacillus plantarum isolates (B1 to B5), B1 Prokka gbk was used as reference for Snippy SNP analysis—standard method. Single comparisons were performed using read data for each strain. B1 reads were ran as a control.

Example command was:
snippy --cpus 24 --outdir B5 --ref B1_Slmod.gbk --pe1
B5_S17_L001_R1_001.fastq.gz --pe2 B5_S17_L001_R2_001.fastq.gz
Calculated individual comparisons and core using B1 gbk as reference
snippy-core --prefix core B1 B2 B3 B4 B5

Comparisons were also performed between B1 and the reference strain read data downloaded from the SRA for Lactobacillus plantarum ATCC 8014 (SRR1552613). Downloading was performed using standard method with prefetch and conversion to fastq using—sratoolkit.2.9.2-win64. Similar approaches were used for comparison of the Leuconostoc mesenteroides isolates BF1 and BF2 with Leuconostoc mesenteroides ATCC 8293 as reference.

### Results

Variants (41) were observed between B1 and ATCC 8014 (Table 13). Variants (1 to 4) were observed between B1 and the other B isolates B2, B3, B4 and B5 (Table 14 to 17). BF1 and BF2 are very different from one another. Variants (19) were observed between BF1 and ATCC 8293 (Table 18). Variants (˜7000) were observed between BF2 and ATCC 8293. 459 complex variants were identified between BF2 and ATCC8293 which are summarized in Table 19.

### Example 17—Short Chain Fatty Acid Assessment in an In Vitro Colonic Fermentation Model

### Samples

Raw: untreated broccoli (blended, and freeze dried into powder with no fermentation). Broccoli florets were homogenized with water (3 parts broccoli to 2 parts of water) for 1 min using a kitchen scale magic bullet blender (Nutribullet pro 900 series, LLC, USA).

Raw fermented: raw broccoli which has been fermented and then freeze dried. Preheat fermented: broccoli that has been subject to a heat pre-treatment prior to fermentation and freeze drying. Broccoli florets were cut at approximately 2 cm below the head and packed in retort pouches, sealed and pre-heated in a thermostated water batch maintained at 65° C. (Core temperature 65° C. for 3 min), and immediately following the heat treatment, the samples were cooled in ice water and homogenised as above and the homogenized samples were incubated in dark for 4 h at 25° C.

TABLE 13
Polymorphisms identified by variant analysis B1 compared to ATCC8014.

| POS | TYPE | REF | ALT | EVIDENCE | FTYPE | STRAND | NT_POS | AA_POS | EFFECT | LOCUS_TAG | GENE |
|---|---|---|---|---|---|---|---|---|---|---|---|
| 292863 | complex | GTCG | ATCT | ATCT:96 GTCG:0 | CDS | + | 292/477 | 98/158 | missense_variant | JBMIHLAL_00290 | ohrR_1 |
| | | | | | | | | | c.292_295delGTCGins | | |
| | | | | | | | | | ATCT p.ValAla98IleSer | | |
| 21413 | snp | C | T | T:204 C:1 | | | | | | | |
| 49138 | snp | T | G | G:226 T:2 | CDS | + | 771/1011 | 257/336 | missense_variant | JBMIHLAL_00337 | lacR_1 |
| | | | | | | | | | c.771T>G p.Asn257Lys | | |
| 68529 | del | TATTAATG | TA | TA:97 | | | | | | | |
| | | GCTCGCGT | | TATTAATGGCTCG | | | | | | | |
| | | CATTAA | | CGTCATTAA:0 | | | | | | | |
| 70435 | snp | G | A | A:199 G:1 | CDS | − | 95/1959 | 32/652 | missense_variant | JBMIHLAL_00352 | lacS_2 |
| | | | | | | | | | c.95C>T p.Thr32Ile | | |
| 70584 | snp | T | C | C:154 T:1 | | | | | | | |
| 71677 | snp | T | C | C:201 T:0 | CDS | − | 209/1029 | 70/342 | missense_variant | JBMIHLAL_00353 | |
| | | | | | | | | | c.209A>G p.Tyr70Cys | | |
| 72030 | del | CGCTCAAC | CG | CG:91 | CDS | − | 978/996 | 320/331 | inframe_deletion | JBMIHLAL_00354 | lacR_3 |
| | | CAGATTAG | | CGCTCAACCAGAT | | | | | c.958_978delCTGGGT | | |
| | | TACCCAG | | TAGTACCCAG:0 | | | | | ACTAATCTGGTTGAG | | |
| | | | | | | | | | p.Leu320_Glu326del | | |
| 136221 | snp | C | A | A:178 C:1 | CDS | − | 559/1272 | 187/423 | missense_variant | JBMIHLAL_00407 | gatC_1 |
| | | | | | | | | | c.559G>T p.Ala187Ser | | |
| 15092 | snp | C | A | A:102 C:1 | | | | | | | |
| 153210 | snp | G | T | T:117 G:1 | CDS | − | 385/1365 | 129/454 | missense_variant | JBMIHLAL_00681 | gabR |
| | | | | | | | | | c.385C>A p.Gln129Lys | | |
| 38124 | snp | C | T | T:264 C:1 | | | | | | | |
| 128067 | snp | G | A | A:261 G:1 | CDS | − | 208/1344 | 70/447 | missense_variant | JBMIHLAL_01118 | yjjP_1 |
| | | | | | | | | | c.208C>T p.Arg70Cys | | |
| 188850 | snp | A | C | C:241 A:0 | CDS | − | 491/1617 | 164/538 | missense_variant | JBMIHLAL_01179 | oppA_2 |
| | | | | | | | | | c.491T>G p.Ile164Ser | | |
| 2322 | snp | A | G | G:107 A:1 | CDS | − | 397/474 | 133/157 | missense_variant | JBMIHLAL_01186 | adcR |
| | | | | | | | | | c.397T>C p.Phe133Leu | | |
| 111662 | ins | CAA | CAAA | CAAA:133 CAA:11 | CDS | + | 10/876 | 4/291 | frameshift_variant | JBMIHLAL_01302 | mntB |
| | | | | | | | | | c.9dupA p.Ser4fs | | |
| 11376 | snp | G | A | A:115 G:0 | CDS | − | 1831/1947 | 611/648 | synonymous_variant | JBMIHLAL_01356 | |
| | | | | | | | | | c.1831C>T | | |
| | | | | | | | | | p.Leu611Leu | | |
| 115510 | snp | G | A | A:199 G:1 | CDS | − | 95/411 | 32/136 | missense_variant | JBMIHLAL_01453 | |
| | | | | | | | | | c.95C>T p.Thr32Ile | | |
| 143457 | snp | G | C | C:264 G:0 | CDS | + | 1122/1416 | 374/471 | synonymous_variant | JBMIHLAL_01479 | pepD |
| | | | | | | | | | c.1122G>C | | |
| | | | | | | | | | p.Val374Val | | |
| 111973 | snp | G | A | A:118 G:1 | CDS | − | 731/1317 | 244/438 | missense_variant | JBMIHLAL_01603 | murA1 |
| | | | | | | | | | c.731C>T p.Ala244Val | | |
| 27553 | snp | C | T | T:104 C:1 | CDS | − | 472/1092 | 158/363 | missense_variant | JBMIHLAL_01677 | wbnH |
| | | | | | | | | | c.472G>A p.Gly158Ser | | |
| 80888 | snp | T | C | C:84 T:0 | CDS | + | 256/258 | 86/85 | stop_lost&splice_region_variant | JBMIHLAL_01727 | ytIR_1 |
| | | | | | | | | | c.256T>C | | |
| | | | | | | | | | p.Ter86Glnext*? | | |
| 133147 | snp | A | C | C:76 A:0 | CDS | − | 443/663 | 148/220 | missense_variant | JBMIHLAL_01777 | yjbM |
| | | | | | | | | | c.443T>G p.Phe148Cys | | |
| 74711 | snp | C | T | T:212 C:1 | CDS | + | 874/1389 | 292/462 | missense_variant | JBMIHLAL_01855 | murF_2 |
| | | | | | | | | | c.874C>T p.Leu292Phe | | |
| 19793 | snp | T | C | C:114 T:1 | CDS | − | 925/1107 | 309/368 | missense_variant | JBMIHLAL_01907 | sigA |
| | | | | | | | | | c.925A>G | | |
| | | | | | | | | | p.Asn309Asp | | |
| 60643 | snp | C | T | T:89 C:1 | CDS | − | 242/1869 | 81/622 | missense_variant | JBMIHLAL_01945 | dnaK |
| | | | | | | | | | c.242G>A p.Ser81Asn | | |
| 10806 | ins | GTTTTTTTT | GTTTTTTTTTG | GTTTTTTTTTG:49 | | | | | | | |
| | | G | | GTTTTTTTTG:1 | | | | | | | |
| 50276 | complex | CG | CACCACCAGG | CACCACCAGGCCG | CDS | − | 341/555 | 114/184 | missense_variant&inframe_insertion | JBMIHLAL_02031 | ribU |
| | | | CCGATTGTGG | ATTGTGGCGA:39 | | | | | c.341delCinsTCGCCAC | | |
| | | | CGA | CG:0 | | | | | AATCGGCCTGGTGGT | | |
| | | | | | | | | | p.Ala114delinsValAla | | |
| | | | | | | | | | ThrIleGlyLeuValVal | | |
| 50325 | snp | A | C | C:99 A:1 | CDS | − | 293/555 | 98/184 | stop_gained c.293T>G | JBMIHLAL_02031 | ribU |
| | | | | | | | | | p.Leu98* | | |
| 64233 | snp | A | G | G:77 A:1 | CDS | − | 2516/2604 | 839/867 | missense_variant | JBMIHLAL_02043 | cIpB |
| | | | | | | | | | c.2516T>C | | |
| | | | | | | | | | p.Val839Ala | | |
| 79046 | snp | G | C | C:140 G:1 | CDS | + | 394/765 | 132/254 | missense_variant | JBMIHLAL_02139 | ygaZ_2 |
| | | | | | | | | | c.394G>C p.Ala132Pro | | |
| 14904 | snp | G | A | A:82 G:0 | CDS | − | 113/876 | 38/291 | missense_variant | JBMIHLAL_02340 | |
| | | | | | | | | | c.113C>T p.Pro38Leu | | |
| 45542 | snp | T | G | G:158 T:0 | CDS | − | 1312/1728 | 438/575 | missense_variant | JBMIHLAL_02365 | pgcA |
| | | | | | | | | | c.1312A>C | | |
| | | | | | | | | | p.Lys438Gln | | |
| 21706 | ins | TAT | TAAT | TAAT:122 TAT:1 | CDS | + | 872/2604 | 291/867 | frameshift_variant | JBMIHLAL_02489 | mprF |
| | | | | | | | | | c.871dupA p.Ile291fs | | |
| 29454 | del | TGA | TA | TA:73 TGA:0 | CDS | + | 94/132 | 32/43 | frameshift_variant | JBMIHLAL_02559 | |
| | | | | | | | | | c.94delG p.Asp32fs | | |
| 27619 | snp | A | G | G:134 A:1 | CDS | − | 78/588 | 26/195 | synonymous_variant | JBMIHLAL_02812 | |
| | | | | | | | | | c.78T>C p.Gly26Gly | | |
| 4360 | snp | C | T | T:96 C:1 | | | | | | | |
| 8851 | del | CGG | CG | CG:117 CGG:0 | CDS | − | 82/513 | 28/170 | frameshift_variant | JBMIHLAL_02963 | tcaR |
| | | | | | | | | | c.82delC p.Pro28fs | | |
| 19068 | del | CTTGCCGA | CT | CT:51 | CDS | + | 154/564 | 52/187 | frameshift_variant | JBMIHLAL_02974 | |
| | | AATTCGAC | | CTTGCCGAAATTC | | | | | c.154_185delGAAATT | | |
| | | AAACAACC | | GACAAACAACCCT | | | | | CGACAAACAACCCTCG | | |
| | | CTCGGATT | | CGGATTGT:0 | | | | | GATTGTTGCC | | |
| | | GT | | | | | | | p.Glu52fs | | |
| 17533 | ins | ATTTTTTG | ATTTTTTTG | ATTTTTTTG:220 | | | | | | | |
| | | | | ATTTTTTG:2 | | | | | | | |

TABLE 14
Polymorphism identified by variant analysis B2 compared to B1.

| POS | TYPE | REF | ALT | EVIDENCE | FTYPE | STRAND | NT_POS | AA_POS | EFFECT | LOCUS_TAG | GENE |
|---|---|---|---|---|---|---|---|---|---|---|---|
| 8417 | snp | C | T | T:105 C:0 | CDS | + | 105/264 | 35/87 | synonymous_variant | JBMIHLAL_02984 | |
| | | | | | | | | | c.105C > T | | |
| | | | | | | | | | p.Asp35Asp | | |

TABLE 15
Polymorphisms identified by variant analysis B3 compared to B1

| POS | TYPE | REF | ALT | EVIDENCE | FTYPE | STRAND | NT_POS | AA_POS | EFFECT | LOCUS_TAG | GENE |
|---|---|---|---|---|---|---|---|---|---|---|---|
| 4326 | del | TATAAAA | TA | TA:31 | | | | | | | |
| | | AAAGCGA | | TATAAAAA | | | | | | | |
| | | CCCCCGT | | AAGCGACC | | | | | | | |
| | | TCATTAA | | CCCGTTCA | | | | | | | |
| | | CGGTGCC | | TTAACGGT | | | | | | | |
| | | GCTCACA | | GCCGCTCA | | | | | | | |
| | | GATCATT | | CAGATCAT | | | | | | | |
| | | ATTAGTG | | TATTAGTG | | | | | | | |
| | | AAAATCA | | AAAATCAC | | | | | | | |
| | | CCCGGCA | | CCGGCA:0 | | | | | | | |
| 8417 | snp | C | T | T:135 C:0 | CDS | + | 105/264 | 35/87 | synonymous_variant | JBMIHLAL_02984 | |
| | | | | | | | | | c.105C>T | | |
| | | | | | | | | | p.Asp35Asp | | |

TABLE 16
Polymorphism identified by variant analysis B4 compared to B1.

| POS | TYPE | REF | ALT | EVIDENCE | FTYPE | STRAND | NT_POS | AA_POS | EFFECT | LOCUS_TAG | GENE |
|---|---|---|---|---|---|---|---|---|---|---|---|
| 8417 | snp | C | T | T:93 C:0 | CDS | + | 105/264 | 35/87 | synonymous_variant | JBMIHLAL_02984 | |
| | | | | | | | | | c.105C > T | | |
| | | | | | | | | | p.Asp35Asp | | |

TABLE 17
Polymorphisms identified by variant analysis B5 compared to B1.

| POS | TYPE | REF | ALT | EVIDENCE | FTYPE | STRAND | NT_POS | AA_POS | EFFECT | LOCUS_TAG | GENE |
|---|---|---|---|---|---|---|---|---|---|---|---|
| 199035 | snp | T | C | C:124 T:0 | CDS | + | 368/1206 | 123/401 | missense_variant c.368T > C | JBMIHLAL_00 | |
| | | | | | | | | | p.Val123Ala | 946 | |
| 143457 | snp | G | C | C:158 G:0 | CDS | + | 1122/1416 | 374/471 | synonymous_variant | JBMIHLAL_01 | pepD |
| | | | | | | | | | c.1122G > C | 479 | |
| | | | | | | | | | p.Val374Val | | |
| 23797 | snp | A | C | C:146 A:0 | CDS | + | 71/666 | 24/221 | missense_variant c.71A > C | JBMIHLAL_02 | immR_1 |
| | | | | | | | | | p.Gln24Pro | 490 | |
| 8417 | snp | C | T | T:131 C:0 | CDS | + | 105/264 | 35/87 | synonymous_variant c.105C > T | JBMIHLAL_02 | |
| | | | | | | | | | p.Asp35Asp | 984 | |

TABLE 18
Polymorphisms identified by variant analysis BF1 compared to ATCC8293.

| POS | TYPE | REF | ALT | EVIDENCE | FTYPE | STRAND | NT_POS | AA_POS | EFFECT | LOCUS_TAG | GENE |
|---|---|---|---|---|---|---|---|---|---|---|---|
| 197592 | del | TGT | TT | TT:178 | | | | | | | |
| | | | | TGT:0 | | | | | | | |
| 269841 | del | TGG | TG | TG:305 | CDS | + | 33/306 | 11/101 | frameshift_variant c.33delG | LEUM_0316 | |
| | | | | TGG:0 | | | | | p.Asn12fs | | |
| 338699 | snp | G | T | T:239 G:0 | CDS | + | 764/1719 | 255/572 | missense_variant c.764G > T | LEUM_0385 | |
| | | | | | | | | | p.Trp255Leu | | |
| 410044 | snp | C | A | A:210 C:0 | CDS | + | 2229/2457 | 743/818 | synonymous_variant | LEUM_0448 | pheT |
| | | | | | | | | | c.2229C > A | | |
| | | | | | | | | | p.Thr743Thr | | |
| 558511 | ins | CAT | CAAT | CAAT:140 | CDS | + | 204/261 | 68/86 | frameshift_variant c.203dupA | LEUM_0587 | |
| | | | | CAT:0 | | | | | p.His68fs | | |
| 559188 | snp | A | G | G:169 A:0 | CDS | + | 601/981 | 201/326 | missense_variant c.601A > G | LEUM_0588 | |
| | | | | | | | | | p.lIe201Val | | |
| 615572 | del | TCC | TC | TC:245 | | | | | | | |
| | | | | TCC:5 | | | | | | | |
| 755527 | snp | A | T | T:196 A:0 | CDS | + | 351/993 | 117/330 | missense_variant c.351A > T | LEUM_0777 | |
| | | | | | | | | | p.Leu117Phe | | |
| 796683 | del | GCC | GC | GC:207 | CDS | + | 2986/3009 | 996/1002 | frameshift_variant c.2986delC | LEUM_0814 | |
| | | | | GCC:0 | | | | | p.Glu997fs | | |
| 953160 | snp | G | T | T:178 G:0 | CDS | + | 805/843 | 269/280 | missense_variant c.805G > T | LEUM_0952 | |
| | | | | | | | | | p.Ala269Ser | | |
| 1009293 | snp | C | A | A:1652 | CDS | + | | | no annotation | LEUM_1009 | |
| | | | | C:171 | | | | | | | |
| 1094250 | snp | T | A | A:188 T:0 | CDS | + | | | no annotation | LEUM_1090 | |
| 1236979 | snp | G | T | T:194 G:1 | | | | | | | |
| 1237016 | del | CAA | CA | CA:183 | | | | | | | |
| | | | | CAA:6 | | | | | | | |
| 1291050 | del | CGT | CT | CT:177 | | | | | | | |
| | | | | CGT:0 | | | | | | | |
| 1600218 | del | AGG | AG | AG:168 | | | | | | | |
| | | | | AGG:2 | | | | | | | |
| 1624087 | ins | GA | GTA | GTA:205 | | | | | | | |
| | | | | GA:0 | | | | | | | |
| 1693283 | snp | T | A | A:247 T:0 | CDS | — | | | no annotation | LEUM_1724 | |
| 1993032 | snp | G | A | A:209 G:0 | CDS | — | | | no annotation | LEUM_2026 | |

TABLE 19
Polymorphisms identified by variant analysis BF2 compared to ATCC8293.

| POS | REF | ALT | EVIDENCE | FTYPE | STRAND | NT_POS | AA_POS | EFFECT | LOCUS_TAG | GENE |
|---|---|---|---|---|---|---|---|---|---|---|
| 1737 | TTCA | ATCC | ATCC:151 TTCA:0 | CDS | + | 63/1137 | 21/378 | synonymous_variant c.63_66delTTCAinsATCC | LEUM_0002 | |
| | | | | | | | | p.IleSer21IleSer | | |
| 11810 | CATG | TATA | TATA:216 CATG:0 | CDS | + | 144/1626 | 48/541 | missense_variant c.144_147delCATGinsTATA | LEUM_0010 | |
| | | | | | | | | p.AsnMet48AsnIle | | |
| 12635 | ACGT | GCGC | GCGC:255 ACGT:0 | CDS | + | 969/1626 | 323/541 | synonymous_variant c.969_972delACGTinsGCGC | LEUM_0010 | |
| | | | | | | | | p.GlnArg323GlnArg | | |
| 20351 | TCT | GCG | GCG:230 TCT:0 | CDS | + | 172/795 | 58/264 | missense_variant c.172_174delTCTinsGCG | LEUM_0017 | |
| | | | | | | | | p.Ser58Ala | | |
| 22033 | AGCTA | GGCTG | GGCTG:2145 | CDS | + | 1047/118 | 349/394 | missense_variant | LEUM_0018 | |
| | | | AGCTA:0 | | | | | c.1047_1051delAGCTAinsGGCTG | | |
| | | | | | | | | p.GluAlaAsn349GluAlaAsp | | |
| 36499 | TATT | CATC | CATC:289 TATT:0 | CDS | + | 564/1062 | 188/353 | synonymous_variant c.564_567delTATrinsCATC | LEUM_0044 | |
| | | | | | | | | p.ArgIle188ArgIle | | |
| 45902 | GTAATGT | CCACATTAC | CCACATTAC:251 | | | | | | | |
| | GA | | GTAATGTGA:0 | | | | | | | |
| 47145 | TAT | TTCAG | TTCAG:241 TAT:0 | | | | | | | |
| 64340 | CTGT | TTGC | TTGC:335 CTGT:0 | CDS | − | 205/915 | 68/304 | missense_variant c.202_205delACAGinsGCAA | LEUM_0076 | |
| | | | | | | | | p.ThrAsp68AlaAsn | | |
| 70144 | GGTATGG | CGTATGGG | CGTATGGGA:233 | | | | | | | |
| | GATGGGA | A | GGTATGGGATGGGA:0 | | | | | | | |
| 75797 | AGAG | GGAT | GGAT:179 AGAG:0 | CDS | + | 51/171 | 17/56 | missense_variant c.51_54delAGAGinsGGAT | LEUM_0091 | |
| | | | | | | | | p.LeuGlu17LeuAsp | | |
| 97951 | TAAT | CAAG | CAAG:197 TAAT:0 | misc_bind- | + | | | no annotation | | |
| | | | | ing | | | | | | |
| 138065 | GGCG | TGCA | TGCA:279 GGCG:0 | CDS | − | 1002/1431 | 333/476 | synonymous_variant | LEUM_0153 | |
| | | | | | | | | c.999_1002delCGCCinsTGCA p.ValAla333ValAla | | |
| 138074 | AUG | GTTC | GTTC:276 ATTG:0 | CDS | − | 993/1431 | 330/476 | synonymous_variant c.990_993delCAATinsGAAC | LEUM_0153 | |
| | | | | | | | | p.ValAsn330ValAsn | | |
| 138092 | AACT | GACC | GACC:278 AACT:0 | CDS | − | 975/1431 | 324/476 | synonymous_variant c.972_975delAGTTinsGGTC | LEUM_0153 | |
| | | | | | | | | p.ProVal324ProVal | | |
| 140746 | GGGT | AGGC | AGGC:196 GGGT:0 | CDS | + | 366/540 | 122/179 | synonymous_variant | LEUM_0156 | |
| | | | | | | | | c.366_369delGGGTinsAGGC p.GluGly122GluGly | | |
| 140797 | CGCC | TGCT | TGCT:208 CGCC:0 | CDS | + | 417/540 | 139/179 | synonymous_variant c.417_420delCGCansTGCT | LEUM_0156 | |
| | | | | | | | | p.AspAla139AspAla | | |
| 142611 | GTT | CTG | CTG:135 GTT:0 | CDS | + | 271/375 | 91/124 | missense_variant c.271_273delGTTinsCTG | LEUM_0158 | |
| | | | | | | | | p.Val91Leu | | |
| 142687 | CAAAAAG | CAAAAAAA | CAAAAAAA:178 | CDS | + | 353/375 | 118/124 | frameshift_variant&missense_variant | LEUM_0158 | |
| | | | CAAAAAG:0 | | | | | c.353delGinsAA p.Ser118fs | | |
| 145324 | CAG | AAA | AAA:292 CAG:0 | CDS | + | 505/1497 | 169/498 | missense_variant c.505_507delCAGinsAAA | LEUM_0161 | gltX |
| | | | | | | | | p.Gln169Lys | | |
| 162834 | TGAT | GGAC | GGAC:260 TGAT:0 | CDS | + | 2400/2481 | 800/826 | missense_variant c.2400_2403delTGATinsGGAC | LEUM_0185 | |
| | | | | | | | | p.AspAsp800GluAsp | | |
| 192260 | ATAAA | GTAAC | GTAAC:301 | CDS | + | 433/768 | 145/255 | missense_variant c.433_437delATAAAMsGTAAC | LEUM_0228 | truA |
| | | | ATAAA:0 | | | | | p.IleAsn145ValThr | | |
| 196751 | CTAT | ATAC | ATAC:138 CTAT:0 | CDS | − | 55/204 | 18/67 | missense_variant c.52_55delATAGinsGTAT | LEUM_0234 | |
| | | | | | | | | p.IleAla18ValSer | | |
| 196918 | AATA | GATG | GATG:246 AATA:0 | | | | | | | |
| 216494 | CACG | TACC | TACC:230 CACG:0 | CDS | + | 108/978 | 36/325 | synonymous_variant c.108_111delCACGinsTACC | LEUM_0256 | nrdF |
| | | | | | | | | p.AspThr36AspThr | | |
| 231792 | ATCTC | GTCTT | GTCTT:235 ATCTC:0 | CDS | + | 553/1728 | 185/575 | missense_variant c.553_557delATCTansGTCTT | LEUM_0276 | |
| | | | | | | | | p.IleSer185ValLeu | | |
| 231812 | GCTC | ACTT | ACTT:229 GCTC:0 | CDS | + | 573/1728 | 191/575 | synonymous_variant c.573_576delGCTCinsACTT | LEUM_0276 | |
| | | | | | | | | p.AlaLeu191AlaLeu | | |
| 234250 | ACTT | CCTG | CCTG:217 ACTT:0 | CDS | + | 336/642 | 112/213 | synonymous_variant c.336_339delACTrinsCCTG | LEUM_0279 | tmk |
| | | | | | | | | p.GlyLeu112GlyLeu | | |
| 242029 | CTAT | TTAC | TTAC:265 CTAT:0 | CDS | − | 664/966 | 221/321 | missense_variant c.661_664delATAGinsGTAA | LEUM_0287 | |
| | | | | | | | | p.IleAla221ValThr | | |
| 244287 | GACT | AACC | AACC:251 GACT:0 | CDS | + | 1436/1962 | 479/653 | missense_variant c.1436_1439delGACTinsAACC | LEUM_0288 | |
| | | | | | | | | p.ArgLeu479LysPro | | |
| 250392 | GGCG | AGCT | AGCT:182 GGCG:0 | CDS | + | 345/1242 | 115/413 | synonymous_variant c.345_348delGGCGinsAGCT | LEUM_0295 | proA |
| | | | | | | | | p.ValAla115ValAla | | |
| 271910 | TTA | CTG | CTG:297 TTA:0 | CDS | + | 358/843 | 120/280 | synonymous_variant c.358_360delTTAinsCTG | LEUM_0318 | |
| | | | | | | | | p.Leu120Leu | | |
| 288308 | ATA | AC | AC:232 ATA:0 | | | | | | | |
| 318676 | GATTAG | AATCAA | AATCAA:121 | CDS | + | 14/306 | 5/101 | missense_variant c.14_19delGATTAGinsAATCAA | LEUM_0366 | |
| | | | GATTAG:0 | | | | | p.GlyLeuVal5GluSerIle | | |
| 341498 | GTTTTTTT | GTTTTTTTTC | GTTTTTTTTC:114 | | | | | | | |
| | TTA | | GTTTTTTTTTA:0 | | | | | | | |
| 359500 | GCAAG | ACAAC | ACAAC:238 | CDS | + | 3034/3540 | 1012/1179 | missense_variant | LEUM_0399 | |
| | | | GCAAG:0 | | | | | c.3034_3038delGCAAGinsACAAC | | |
| | | | | | | | | p.AlaSer1012ThrThr | | |
| 366821 | ACATC | GCATT | GCATT:250 ACATC:0 | CDS | + | 957/1488 | 319/495 | synonymous_variant | LEUM_0406 | lysS |
| | | | | | | | | c.957_961delACATCinsGCATT | | |
| | | | | | | | | p.LysHisLeu319LysHisLeu | | |
| 366884 | AGAAGCA | GGATGCG | GGATGCG:217 | CDS | + | 1020/1488 | 340/495 | missense_variant | LEUM_0406 | lysS |
| | | | AGAAGCA:0 | | | | | c.1020_1026delAGAAGCAinsGGATGCG | | |
| | | | | | | | | p.GluGluAla340GluAspAla | | |
| 366896 | GTTGGCC | ATTAGCA | ATTAGCA:225 | CDS | + | 1032/1488 | 344/495 | synonymous_variant | LEUM_0406 | lysS |
| | | | GTTGGCC:0 | | | | | c.1032_1038delGTIGGCCinsATTAGCA | | |
| | | | | | | | | p.LysLeuAla344LysLeuAla | | |
| 366971 | ATTTGTA | GTTCGTT | GTTCGTT:225 | CDS | + | 1107/1488 | 369/495 | synonymous_variant | LEUM_0406 | lysS |
| | | | ATTTGTA:0 | | | | | c.1107_1113delATTTGTAinsGTTCGTT | | |
| | | | | | | | | p.GluPheVal369GluPheVal | | |
| 371223 | CTTC | ATTT | ATTT:226 CTTC:0 | CDS | + | 273/1449 | 91/482 | synonymous_variant c.273_276delCTTCinsATTT | LEUM_0414 | |
| | | | | | | | | p.GlyPhe91GlyPhe | | |
| 395520 | CTCT | ATCC | ATCC:206 CTCT:0 | CDS | − | 525/942 | 174/313 | missense_variant c.522_525delAGAGinsGGAT | LEUM_0436 | |
| | | | | | | | | p.IleGlu174MetAsp | | |
| 395821 | ACCA | GCCG | GCCG:177 ACCA:0 | CDS | − | 224/942 | 74/313 | missense_variant c.221_224delTGGTinsCGGC | LEUM_0436 | |
| | | | | | | | | p.MetVal74ThrAla | | |
| 410847 | CGGT | TGGC | TGGC:232 CGGT:0 | CDS | + | 495/1287 | 165/428 | synonymous_variant c.495_498delCGGTinsTGGC | LEUM_0449 | |
| | | | | | | | | p.ValGly165ValGly | | |
| 420486 | CGCAC | AGCAT | AGCAT:187 | CDS | + | 200/609 | 67/202 | missense_variant c.200_204delCGCACinsAGCAT | LEUM_0457 | |
| | | | CGCAC:0 | | | | | p.AlaHis67GluHis | | |
| 455735 | GTG | CTT | CTT:112 GTG:0 | CDS | − | 1922/2088 | 640/695 | missense_variant c.1920_1922delCACinsAAG | LEUM_0497 | |
| | | | | | | | | p.AsnThr640LysSer | | |
| 457087 | GCCAT | ACCAC | ACCAC:262 | CDS | − | 570/2088 | 189/695 | missense_variant c.566_570delATGGansGTGGT | LEUM_0497 | |
| | | | GCCAT:0 | | | | | p.AspGly189GlyGly | | |
| 490235 | GCG | ACA | ACA:136 GCG:0 | CDS | + | 142/738 | 48/245 | missense_variant c.142_144delGCGinsACA | LEUM_0524 | |
| | | | | | | | | p.Ala48Thr | | |
| 493487 | TGGT | CGGC | CGGC:189 TGGT:0 | CDS | + | 168/834 | 56/277 | synonymous_variant c.168_171delTGGTinsCGGC | LEUM_0527 | |
| | | | | | | | | p.ArgGly56ArgGly | | |
| 500830 | GCT | ACC | ACC:176 GCT:0 | CDS | + | 352/2031 | 118/676 | missense_variant c.352_354delGCTinsACC | LEUM_0536 | |
| | | | | | | | | p.Ala118Thr | | |
| 502254 | CGAA | TGAG | TGAG:214 CGAA:0 | CDS | + | 1776/2031 | 592/676 | synonymous_variant | LEUM_0536 | |
| | | | | | | | | c.1776_1779delCGAAinsTGAG | | |
| | | | | | | | | p.ValGIu592ValGlu | | |
| 502272 | CATTC | TCTCT | TCTCT:187 CATTC:0 | CDS | + | 1794/2031 | 598/676 | missense_variant | LEUM_0536 | |
| | | | | | | | | c.1794_1798delCATTCinsTCTCT | | |
| | | | | | | | | p.PheIleLeu598PheLeuLeu | | |
| 502291 | TTG | CTA | CTA:215 TTG:0 | CDS | + | 1813/2031 | 605/676 | synonymous_variant c.1813_1815delTTGinsCTA | LEUM_0536 | |
| | | | | | | | | p.Leu605Leu | | |
| 505441 | AGG | GGA | GGA:156 AGG:4 | CDS | + | 826/834 | 276/277 | missense_variant c.826_828delAGGinsGGA | LEUM_0540 | |
| | | | | | | | | p.Arg276Gly | | |
| 507015 | ACCAC | GCCAA | GCCAA:199 | CDS | − | 507/1098 | 168/365 | missense_variant c.503_507delGTGGTinsTTGGC | LEUM_0543 | |
| | | | ACCAC:0 | | | | | p.SerGly168IleGly | | |
| 508582 | TGCT | CGCG | CGCG:163 TGCT:0 | CDS | + | 861/1008 | 287/335 | synonymous_variant c.861_864delTGCTinsCGCG | LEUM_0544 | |
| | | | | | | | | p.ProAla287ProAla | | |
| 509588 | TTG | CTA | CTA:171 TTG:0 | CDS | + | 751/1866 | 251/621 | synonymous_variant c.751_753delTrGinsCTA | LEUM_0545 | |
| | | | | | | | | p.Leu251Leu | | |
| 510386 | GTCATA | ATCTTG | ATCTTG:158 | CDS | + | 1549/1866 | 517/621 | missense_variant | LEUM_0545 | |
| | | | GTCATA:0 | | | | | c.1549_1554delGTCATAinsATCTTG | | |
| | | | | | | | | p.ValIle517IleLeu | | |
| 511743 | CAGC | AAGT | AAGT:187 CAGC:0 | CDS | + | 927/1347 | 309/448 | synonymous_variant c.927_930delCAGansAAGT | LEUM_0546 | |
| | | | | | | | | p.LeuSer309LeuSer | | |
| 519040 | TCGT | CCGC | CCGC:165 TCGT:0 | CDS | + | 210/1371 | 70/456 | synonymous_variant c.210_213delTCGTinsCCGC | LEUM_0553 | |
| | | | | | | | | p.GlyArg70GlyArg | | |
| 530354 | TTGG | GTGA | GTGA:118 TTGG:0 | CDS | + | 193/1728 | 65/575 | missense_variant c.193_196delTTGGinsGTGA | LEUM_0562 | |
| | | | | | | | | p.LeuVal65ValMet | | |
| 536863 | AAGA | GAGG | GAGG:178 AAGA:0 | CDS | + | 1959/2301 | 653/766 | synonymous_variant | LEUM_0566 | |
| | | | | | | | | c.1959_1962delAAGAinsGAGG | | |
| | | | | | | | | p.SerArg653SerArg | | |
| 560132 | AAC | TAT | TAT:202 AAC:0 | CDS | + | 423/882 | 141/293 | missense_variant c.423_425delAACinsTAT | LEUM_0589 | |
| | | | | | | | | p.ValThr141ValMet | | |
| 603339 | AAT | GAC | GAC:238 AAT:0 | CDS | + | 673/1944 | 225/647 | missense_variant c.673_675delAATinsGAC | LEUM_0636 | |
| | | | | | | | | p.Asn225Asp | | |
| 607531 | GAGC | AAGT | AAGT:217 GAGC:0 | CDS | + | 438/894 | 146/297 | missense_variant c.438_441delGAGCMsAAGT | LEUM_0640 | |
| | | | | | | | | p.MetSer146IleSer | | |
| 610263 | TAACA | CAACG | CAACG:174 | CDS | + | 773/1464 | 258/487 | missense_variant c.773_777delTAACAMsCAACG | LEUM_0643 | |
| | | | TAACA:0 | | | | | p.LeuThr258SerThr | | |
| 610344 | TAG CTGC | CAGCTGCAA | CAGCTGCAAGTG:127 | CDS | + | 854/1464 | 285/487 | missense_variant&inframe_deletion | LEUM_0643 | |
| | AAGTGCT | GTG | TAGCTGCAAGTGCT | | | | | c.854_864delTAGCTGCAAGTinsCA | | |
| | GCAAGTG | | GCAAGTG:0 | | | | | p.Ile285_Ser288delinsThr | | |
| 613023 | CGGC | AGGT | AGGT:209 CGGC:0 | CDS | + | 801/1143 | 267/380 | synonymous_variant c.801_804delCGGCinsAGGT | LEUM_0645 | |
| | | | | | | | | p.ProGly267ProGly | | |
| 613326 | GACG | AACA | AACA:160 GACG:0 | CDS | + | 1104/1143 | 368/380 | synonymous_variant | LEUM_0645 | |
| | | | | | | | | c.1104_1107delGACGinsAACA | | |
| | | | | | | | | p.AlaThr368AlaThr | | |
| 615534 | GTTG | ATTA | ATTA:217 GTTG:0 | | | | | | | |
| 615580 | GCCC | CCCT | CCCT:199 GCCC:0 | | | | | | | |
| 641900 | TCCG | CCCA | CCCA:199 TCCG:0 | CDS | + | 417/570 | 139/189 | synonymous_variant c.417_420delTCCGinsCCCA | LEUM_0673 | |
| | | | | | | | | p.TyrPro139TyrPro | | |
| 642442 | CAGTA | TAGCG | TAGCG:148 | CDS | + | 282/684 | 94/227 | missense_variant c.282_286delCAGTAinsTAGCG | LEUM_0674 | |
| | | | CAGTA:0 | | | | | p.GlySerThr94GlySerAla | | |
| 654478 | CTTC | TTTT | TTTT:217 CTTC:0 | CDS | + | 597/795 | 199/264 | synonymous_variant c.597_600delCTTCinsTTTT | LEUM_0686 | |
| | | | | | | | | p.AsnPhe199AsnPhe | | |
| 658429 | TCG | GCA | GCA:147 TCG:0 | CDS | + | 622/4314 | 208/1437 | missense_variant c.622_624delTCGinsGCA | LEUM_0689 | |
| | | | | | | | | p.Ser208Ala | | |
| 671357 | CAGTTAT | AAGCTAC | AAGCTAC:180 | CDS | + | 432/891 | 144/296 | synonymous_variant | LEUM_0698 | |
| | | | CAGTTAT:0 | | | | | c.432_438delCAGTTATinsAAGCTAC | | |
| | | | | | | | | p.LeuSerTyr144LeuSerTyr | | |
| 697054 | AAT | CAG | CAG:204 AAT:0 | CDS | + | 2160/2217 | 720/738 | missense_variant c.2160_2162delAATinsCAG | LEUM_0723 | |
| | | | | | | | | p.LeuIle720PheSer | | |
| 700692 | ACCC | CCCT | CCCT:206 ACCC:0 | CDS | + | 378/1527 | 126/508 | synonymous_variant c.378_381delACCCinsCCCT | LEUM_0727 | purH |
| | | | | | | | | p.GlyPro126GlyPro | | |
| 700713 | AGCT | TGCC | TGCC:209 AGCT:0 | CDS | + | 399/1527 | 133/508 | synonymous_variant c.399_402delAGCTinsTGCC | LEUM_0727 | purH |
| | | | | | | | | p.AlaAla133AlaAla | | |
| 701025 | CGGCAAA | TGGTAAG | TGGTAAG:121 | CDS | + | 711/1527 | 237/508 | synonymous_variant | LEUM_0727 | purH |
| | | | CGGCAAA:0 | | | | | c.711_717delCGGCAAAinsTGGTAAG | | |
| | | | | | | | | p.HisGlyLys237HisGlyLys | | |
| 723536 | CACTG | TACTC | TACTC:162 CACTG:0 | CDS | + | 326/534 | 109/177 | missense_variant c.326_330delCACTGinsTACTC | LEUM_0746 | |
| | | | | | | | | p.ThrLeu109IleLeu | | |
| 726007 | ATAAA | TTTAT | TTTAT:130 ATAAA:0 | | | | | | | |
| 745561 | ATAAT | GTAAC | GTAAC:87 ATAAT:0 | | | | | | | |
| 751089 | ACTG | GCTA | GCTA:157 ACTG:0 | CDS | + | 2232/3339 | 744/1112 | synonymous_variant | LEUM_0774 | |
| | | | | | | | | c.2232_2235delACTGinsGCTA | | |
| | | | | | | | | p.GluLeu744GluLeu | | |
| 769650 | GCCA | ACCG | ACCG:139 GCCA:0 | CDS | − | 27/834 | 8/277 | synonymous_variant c.24_27delTGGCinsCGGT | LEUM_0791 | |
| | | | | | | | | p.AspGly8AspGly | | |
| 784937 | CCCG | TCCA | TCCA:96 CCCG:0 | CDS | − | 1608/1674 | 535/557 | synonymous_variant | LEUM_0807 | |
| | | | | | | | | c.1605_1608delCGGGinsTGGA p.IleGly535IleGly | | |
| 787928 | AAACG | GAACC | GAACC:132 | CDS | + | 1190/1701 | 397/566 | missense_variant | LEUM_0808 | |
| | | | AAACG:0 | | | | | c.1190_1194delAAACGinsGAACC | | |
| | | | | | | | | p.GlnThr397ArgThr | | |
| 788232 | TATCATC | CATCTTG | CATCTTG:120 | CDS | + | 1494/1701 | 498/566 | missense_variant | LEUM_0808 | |
| | | | TATCATC:0 | | | | | c.1494_1500delTATCATCinsCATCTTG | | |
| | | | | | | | | p.ThrlIeIle498ThrIleLeu | | |
| 796989 | ATTAGGC | GCTGGGT | GCTGGGT:149 | | | | | | | |
| | | | ATTAGGC:0 | | | | | | | |
| 797082 | GGGA | TGGG | TGGG:154 GGGA:0 | | | | | | | |
| 797274 | TAAAA | GAAAC | GAAAC:136 | | | | | | | |
| | | | TAAAA:0 | | | | | | | |
| 800184 | ACAAT | GCAAG | GCAAG:171 | CDS | + | 900/4521 | 300/1506 | missense_variant c.900_904delACAATinsGCAAG | LEUM_0818 | |
| | | | ACAAT:0 | | | | | p.ProGlnSer300ProGlnAla | | |
| 829273 | CATTAT | AAGTAC | AAGTAC:116 | CDS | + | 211/909 | 71/302 | missense_variant | LEUM_0842 | |
| | | | CATTAT:0 | | | | | c.211_216delCATTATinsAAGTAC | | |
| | | | | | | | | p.HisTyr7lLysTyr | | |
| 831087 | TAGC | CAAT | CAAT:103 TAGC:0 | CDS | − | 408/897 | 135/298 | synonymous_variant c.405_408delGCTAinsATTG | LEUM_0844 | |
| | | | | | | | | p.ValLeu135ValLeu | | |
| 831917 | GAACAGG | AAACCGGC | AAACCGGC:130 | CDS | + | 300/2025 | 100/674 | synonymous_variant | LEUM_0845 | |
| | T | | GAACAGGT:0 | | | | | c.300_307delGAACAGGTinsAAACCGGC | | |
| | | | | | | | | p.GlyAsnArgLeu100GlyAsnArgLeu | | |
| 832789 | GAGC | CAGT | CAGT:158 GAGC:0 | CDS | + | 1172/2025 | 391/674 | missense_variant c.1172_1175delGAGCinsCAGT | LEUM_0845 | |
| | | | | | | | | p.GlyAla391AlaVal | | |
| 833573 | TATGG | CATGA | CATGA:172 | CDS | + | 1956/2025 | 652/674 | missense_variant | LEUM_0845 | |
| | | | TATGG:0 | | | | | c.1956_1960delTATGGinsCATGA | | |
| | | | | | | | | p.HisMetAla652HisMetThr | | |
| 835366 | GCAT | ACAA | ACAA:139 GCAT:0 | CDS | + | 459/1149 | 153/382 | missense_variant c.459_462delGCATinsACAA | LEUM_0847 | |
| | | | | | | | | p.GlyHis153GlyGln | | |
| 838604 | AAGT | GAGC | GAGC:132 AAGT:0 | CDS | + | 687/729 | 229/242 | synonymous_variant c.687_690delAAGTinsGAGC | LEUM_0849 | |
| | | | | | | | | p.GlySer229GlySer | | |
| 838832 | GGTAC | AGCAT | AGCAT:131 | CDS | + | 185/330 | 62/109 | missense_variant c.185_189delGGTACinsAGCAT | LEUM_0850 | |
| | | | GGTAC:0 | | | | | p.GlyTyr62GluHis | | |
| 843675 | CAGATTA | AAAATCAAA | AAAATCAAAA:133 | CDS | + | 256/1620 | 86/539 | missense_variant | LEUM_0854 | |
| | ACG | A | CAGATTAACG:0 | | | | | c.256_265delCAGATTAACGinsAAAATCAAAA | | |
| | | | | | | | | p.GlnIleAsnAla86LysIleLysThr | | |
| 843731 | GAAT | AAAC | AAAC:158 GAAT:0 | CDS | + | 312/1620 | 104/539 | synonymous_variant c.312_315delGAATinsAAAC | LEUM_0854 | |
| | | | | | | | | p.LysAsn104LysAsn | | |
| 847585 | AACA | GACG | GACG:149 AACA:0 | CDS | + | 660/8466 | 220/2821 | synonymous_variant c.660_663delAACAinsGACG | LEUM_0857 | |
| | | | | | | | | p.ThrThr220ThrThr | | |
| 853659 | ATA | GTG | GTG:201 ATA:0 | CDS | + | 6734/8466 | 2245/2821 | missense_variant c.6734_6736delATAinsGTG | LEUM_0857 | |
| | | | | | | | | p.AsnAsn2245SerAsp | | |
| 863407 | GTAA | TTGC | TTGC:77 GTAA:0 | | | | | | | |
| 870920 | TC | TAT | TAT:106 TC:0 | | | | | | | |
| 876892 | ATAGCTC | CTAGATCG | CTAGATCG:171 | CDS | + | 367/2223 | 123/740 | missense_variant | LEUM_0882 | |
| | A | | ATAGCTCA:0 | | | | | c.367_374delATAGCTCAinsCTAGATCG | | |
| | | | | | | | | p.IleAlaHis123LeuAspArg | | |
| 877704 | CGCC | TGCT | TGCT:185 CGCC:0 | CDS | + | 1179/2223 | 393/740 | synonymous_variant | LEUM_0882 | |
| | | | | | | | | c.1179_1182delCGCCinsTGCT p.TyrAla393TyrAla | | |
| 880042 | ACTAT | TCTAC | TCTAC:151 ACTAT:0 | CDS | + | 77/1506 | 26/501 | missense_variant c.77_81delACTATinsTCTAC | LEUM_0884 | |
| | | | | | | | | p.AsnTyr26IleTyr | | |
| 883034 | ACCACTT | GCCGCTC | GCCGCTC:136 | CDS | + | 1422/2253 | 474/750 | missense_variant | LEUM_0885 | |
| | | | ACCACTT:0 | | | | | c.1422_1428delACCACTTinsGCCGCTC | | |
| | | | | | | | | p.IleProLeu474MetProLeu | | |
| 883123 | GAGA | AAGG | AAGG:126 GAGA:0 | CDS | + | 1511/2253 | 504/750 | missense_variant c.1511_1514delGAGAinsAAGG | LEUM_0885 | |
| | | | | | | | | p.ArgGlu504LysGly | | |
| 893725 | TAA | CAG | CAG:132 TAA:0 | CDS | + | 1167/2259 | 389/752 | missense_variant c.1167_1169delTAAinsCAG | LEUM_0894 | |
| | | | | | | | | p.AlaLys389AlaArg | | |
| 894794 | AAA | GAG | GAG:173 AAA:0 | CDS | + | 2236/2259 | 746/752 | missense_variant c.2236_2238delAAAinsGAG | LEUM_0894 | |
| | | | | | | | | p.Lys746Glu | | |
| 895508 | CAAG | TAAA | TAAA:112 CAAG:0 | CDS | + | 675/687 | 225/228 | synonymous_variant c.675_678delCAAGinsTAAA | LEUM_0895 | |
| | | | | | | | | p.IleLys225IleLys | | |
| 895583 | ATTAAGC | GTCAAGTT | GTCAAGTT:92 | CDS | − | 996/1008 | 330/335 | missense_variant | LEUM_0896 | |
| | G | | ATTAAGCG:0 | | | | | c.989_996delCGCTTAATinsAACTTGAC | | |
| | | | | | | | | p.ThrLeuAsn330LysLeuAsp | | |
| 895607 | CGGT | TGGG | TGGG:101 CGGT:0 | CDS | − | 972/1008 | 323/335 | synonymous_variant c.969_972delACCGinsCCCA | LEUM_0896 | |
| | | | | | | | | p.ValPro323ValPro | | |
| 903892 | CTTTGCCT | TTTTACCTC | TTTTACCTC:158 | CDS | + | 1215/1839 | 405/612 | missense_variant | LEUM_0901 | |
| | T | | CTTTGCCTT:0 | | | | | c.1215_1223delCTTTGCCTTinsTTTTACCTC | | |
| | | | | | | | | p.AlaPheAlaLeu405AlaPheThrSer | | |
| 907285 | GCTAC | ACTAT | ACTAT:127 GCTAC:0 | | | | | | | |
| 911930 | CAGC | TAGT | TAGT:94 CAGC:0 | CDS | + | 39/822 | 13/273 | synonymous_variant c.39_42delCAGCinsTAGT | LEUM_0909 | |
| | | | | | | | | p.SerSer13SerSer | | |
| 933210 | CAGGGC | GAGCGT | GAGCGT:156 | CDS | + | 1909/1992 | 637/663 | missense_variant | LEUM_0929 | |
| | | | CAGGGC:0 | | | | | c.1909_1914delCAGGGCinsGAGCGT | | |
| | | | | | | | | p.GlnGly637GluArg | | |
| 945839 | TAG | TAAA | TAAA:60 TAG:0 | | | | | | | |
| 945853 | GAT | AAC | AAC:61 GAT:0 | | | | | | | |
| 972869 | CATT | TATC | TATC:142 CATT:0 | CDS | + | 168/480 | 56/159 | synonymous_variant c.168_171delCATTinsTATC | LEUM_0972 | |
| | | | | | | | | p.HisIle56HisIle | | |
| 980203 | TTAGTA | CTGGTG | CTGGTG:85 | CDS | + | 220/513 | 74/170 | synonymous_variant | LEUM_0980 | |
| | | | TTAGTA:0 | | | | | c.220_225delTTAGTAinsCTGGTG | | |
| | | | | | | | | p.LeuVal74LeuVal | | |
| 980531 | TCATTA | CAATTG | CAATTG:125 | | | | | | | |
| | | | TCATTA:0 | | | | | | | |
| 982914 | AGCT | GGCA | GGCA:58 AGCT:0 | CDS | + | | | no annotation | LEUM_0984 | |
| 986252 | GGTCC | TGTCT | TGTCT:31 GGTCC:0 | CDS | + | | | no annotation | LEUM_0987 | |
| 986279 | CGAAACG | TGAGACACT | TGAGACACTAATTA:30 | CDS | + | | | no annotation | LEUM_0987 | |
| | CTCATTC | AATTA | CGAAACGCTCATTC:0 | | | | | | | |
| 986308 | GGTC | AGAT | AGAT:30 GGTC:0 | | | | | | | |
| 986319 | ATT | GTC | GTC:31 ATT:0 | CDS | + | | | no annotation | LEUM_0988 | |
| 986356 | CGTT | TGTG | TGTG:30 CGTT:0 | CDS | + | | | no annotation | LEUM_0988 | |
| 986375 | GTTTCAG | ATGTCGGA | ATGTCGGAAGAG:25 | CDS | + | | | no annotation | LEUM_0988 | |
| | AAAAA | AGAG | GTTTCAGAAAAA:0 | | | | | | | |
| 1008480 | CAAG | TAAA | TAAA:14 CAAG:0 | CDS | + | | | no annotation | LEUM_1008 | |
| 1008786 | CCTG | TCTA | TCTA:1619 CCTG:0 | CDS | + | | | no annotation | LEUM_1009 | |
| 1008954 | ACCC | GCCA | GCCA:1877 ACCC:0 | CDS | + | | | no annotation | LEUM_1009 | |
| 1022214 | TTTG | ATTA | ATTA:76 TTTG:0 | | | | | | | |
| 1135118 | TGG | CGA | CGA:83 TGG:0 | | | | | | | |
| 1135159 | TCGT | CCGC | CCGC:83 TCGT:0 | | | | | | | |
| 1135269 | TTAC | CTAT | CTAT:123 TTAC:0 | CDS | + | | | no annotation | LEUM_1138 | |
| 1138281 | GTTT | ATTC | ATTC:201 GTTT:0 | CDS | − | | | no annotation | LEUM_1142 | |
| 1139585 | CAACC | TAACT | TAACT:197 CAACC:0 | CDS | − | | | no annotation | LEUM_1143 | |
| 1155368 | AGCG | GGCA | GGCA:141 AGCG:0 | CDS | − | | | no annotation | LEUM_1157 | |
| 1157871 | ATTT | GTTG | GTTG:155 ATTT:0 | CDS | − | | | no annotation | LEUM_1161 | |
| 1169465 | GTCG | TTCT | TTCT:178 GTCG:0 | CDS | − | | | no annotation | LEUM_1172 | |
| 1170652 | GCG | TCA | TCA:135 GCG:0 | CDS | − | | | no annotation | LEUM_1173 | |
| 1170669 | TATC | CATT | CATT:124 TATC:0 | CDS | − | | | no annotation | LEUM_1173 | |
| 1170980 | TTTA | CTCG | CTCG:123 TTTA:0 | CDS | − | | | no annotation | LEUM_1174 | |
| 1174201 | GAC | AAT | AAT:87 GAC:0 | | | | | | | |
| 1174261 | CGTG | AGTA | AGTA:130 CGTG:0 | CDS | − | | | no annotation | LEUM_1177 | |
| 1183816 | GGTA | AGTG | AGTG:139 GGTA:0 | CDS | − | | | no annotation | LEUM_1187 | |
| 1194019 | GCAAT | ACAAC | ACAAC:139 | CDS | − | | | no annotation | LEUM_1195 | |
| | | | GCAAT:0 | | | | | | | |
| 1238393 | GGCAGG | AGTAGA | AGTAGA:81 | | | | | | | |
| | | | GGCAGG:0 | | | | | | | |
| 1238441 | TAAT | GATA | GATA:47 TAAT:0 | | | | | | | |
| 1258437 | CTT | TTG | TTG:43 CTT:0 | | | | | | | |
| 1263043 | TGGG | CGGA | CGGA:194 TGGG:0 | CDS | + | | | no annotation | LEUM_1275 | |
| 1267583 | TGGGCAG | GGGTCAA | GGGTCAA:131 | CDS | + | | | no annotation | LEUM_1279 | |
| | | | TGGGCAG:0 | | | | | | | |
| 1289296 | TCTC | CCTT | CCTT:197 TCTC:0 | CDS | − | | | no annotation | LEUM_1302 | |
| 1294486 | ACAA | GCA | GCA:189 ACAA:0 | | | | | | | |
| 1296449 | CAGCTGT | TATCCGTG | TATCCGTG:188 | CDS | − | | | no annotation | LEUM_1309 | aspS |
| | A | | CAGCTGTA:0 | | | | | | | |
| 1302442 | TCCG | ACCA | ACCA:161 TCCG:0 | CDS | − | | | no annotation | LEUM_1314 | |
| 1303222 | AGTA | GGTG | GGTG:220 AGTA:0 | CDS | − | | | no annotation | LEUM_1314 | |
| 1306063 | TACC | GACA | GACA:193 TACC:0 | CDS | − | | | no annotation | LEUM_1316 | lacZ |
| 1319219 | TACAGCA | CACATCAC | CACATCAC:135 | | | | | | | |
| | A | | TACAGCAA:0 | | | | | | | |
| 1319558 | ATTTAAGT | CTACAATAT | CTACAATATCACTTC | | | | | | | |
| | TCAGTCA | CACTTCCC | CC:109 | | | | | | | |
| | CA | | ATTTAAGTTCAGTCA | | | | | | | |
| | | | CA:0 | | | | | | | |
| 1319611 | ACGTCT | CCGTTC | CCGTTC:146 | | | | | | | |
| | | | ACGTCT:0 | | | | | | | |
| 1319951 | ACGC | GCGT | GCGT:150 ACGC:0 | CDS | + | | | no annotation | LEUM_1334 | |
| 1345228 | ACTTG | GCTTA | GCTTA:204 ACTTG:0 | CDS | − | | | no annotation | LEUM_1363 | |
| 1346846 | TGGG | CGGA | CGGA:191 TGGG:0 | CDS | − | | | no annotation | LEUM_1363 | |
| 1392214 | TAAA | AAGC | AAGC:157 TAAA:0 | CDS | − | | | no annotation | LEUM_1404 | |
| 1396399 | CGC | TGT | TGT:177 CGC:0 | CDS | − | | | no annotation | LEUM_1408 | |
| 1407216 | TGA | AGC | AGC:120 TGA:0 | CDS | − | | | no annotation | LEUM_1412 | |
| 1407234 | TGTTAGT | AGCTAAC | AGCTAAC:94 | CDS | − | | | no annotation | LEUM_1412 | |
| | | | TGTTAGT:0 | | | | | | | |
| 1407252 | AATG | GATA | GATA:112 AATG:0 | CDS | − | | | no annotation | LEUM_1412 | |
| 1410440 | GCTT | ACTC | ACTC:158 GCTT:0 | CDS | − | | | no annotation | LEUM_1415 | |
| 1410471 | CTT | ATC | ATC:162 CTT:0 | CDS | − | | | no annotation | LEUM_1415 | |
| 1415069 | TTTC | CTTA | CTTA:140 TTTC:0 | CDS | − | | | no annotation | LEUM_1420 | |
| 1415084 | CACT | AACA | AACA:142 CACT:0 | CDS | − | | | no annotation | LEUM_1420 | |
| 1415294 | AAGT | TAGC | TAGC:163 AAGT:0 | CDS | − | | | no annotation | LEUM_1420 | |
| 1415654 | GTAC | ATAA | ATAA:203 GTAC:0 | CDS | − | | | no annotation | LEUM_1420 | |
| 1415711 | AGCT | CGCC | CGCC:184 AGCT:0 | CDS | − | | | no annotation | LEUM_1420 | |
| 1415881 | AAC | GAA | GAA:192 AAC:0 | | | | | | | |
| 1416065 | GCCT | TCCA | TCCA:207 GCCT:0 | CDS | − | | | no annotation | LEUM_1421 | |
| 1416263 | GTTT | ATTA | ATTA:191 GTTT:0 | CDS | − | | | no annotation | LEUM_1421 | |
| 1416317 | GATG | AATA | AATA:199 GATG:0 | CDS | − | | | no annotation | LEUM_1421 | |
| 1416380 | CAAA | TAAG | TAAG:211 CAAA:0 | CDS | − | | | no annotation | LEUM_1421 | |
| 1416695 | TGTT | GGTC | GGTC:168 TGTT:0 | CDS | − | | | no annotation | LEUM_1421 | |
| 1417341 | ATTG | GTTA | GTTA:195 ATTG:0 | CDS | − | | | no annotation | LEUM_1422 | |
| 1417434 | ATTA | GTTG | GTTG:217 ATTA:0 | CDS | − | | | no annotation | LEUM_1422 | |
| 1417596 | CAG | TAA | TAA:222 CAG:0 | CDS | − | | | no annotation | LEUM_1423 | |
| 1417722 | AAGGAGA | GAGAAGT | GAGAAGT:134 | CDS | − | | | no annotation | LEUM_1423 | |
| | | | AAGGAGA:0 | | | | | | | |
| 1417734 | CAACGTT | GTGTGTC | GTGTGTC:128 | CDS | − | | | no annotation | LEUM_1423 | |
| | | | CAACGTT:0 | | | | | | | |
| 1417782 | GTCT | ATCC | ATCC:185 GTCT:0 | CDS | − | | | no annotation | LEUM_1423 | |
| 1417965 | CTTGTCA | TTTATCG | TTTATCG :206 | CDS | − | | | no annotation | LEUM_1423 | |
| | | | CTTGTCA:0 | | | | | | | |
| 1418013 | GCCA | ACCG | ACCG:208 GCCA:0 | CDS | − | | | no annotation | LEUM_1423 | |
| 1418025 | GGCG | AGCA | AGCA:180 GGCG:0 | CDS | − | | | no annotation | LEUM_1423 | |
| 1418040 | TAAAGCC | CAGAGCAG | CAGAGCAGCTTC:88 | CDS | − | | | no annotation | LEUM_1423 | |
| | TCTTG | CTTC | TAAAGCCTCTTG:0 | | | | | | | |
| 1418061 | TTG | CTC | CTC:91 TTG:0 | CDS | − | | | no annotation | LEUM_1423 | |
| 1418069 | GACCGGC | ACCCTGCG | ACCCTGCG:89 | CDS | − | | | no annotation | LEUM_1423 | |
| | A | | GACCGGCA:0 | | | | | | | |
| 1418094 | TCCC | ACCT | ACCT:100 TCCC:0 | CDS | − | | | no annotation | LEUM_1423 | |
| 1418103 | TAAG | CAGA | CAGA:87 TAAG:0 | CDS | − | | | no annotation | LEUM_1423 | |
| 1418148 | CGCG | TGCA | TGCA:197 CGCG:0 | CDS | − | | | no annotation | LEUM_1423 | |
| 1418160 | GCCA | ACCG | ACCG:194 GCCA:0 | CDS | − | | | no annotation | LEUM_1423 | |
| 1418193 | GTGCAA | ATTTAG | ATTTAG:162 | CDS | − | | | no annotation | LEUM_1423 | |
| | | | GTGCAA:0 | | | | | | | |
| 1418208 | ATGG | CTGA | CTGA:175 ATGG:0 | CDS | − | | | no annotation | LEUM_1423 | |
| 1418271 | TTTT | ATCC | ATCC:170 TTTT:0 | CDS | − | | | no annotation | LEUM_1423 | |
| 1418322 | TTTA | CTTG | CTTG:167 TTTA:0 | CDS | − | | | no annotation | LEUM_1423 | |
| 1418385 | AGAG | GGAA | GGAA:118 AGAG:0 | CDS | − | | | no annotation | LEUM_1423 | |
| 1418582 | ACC | GCT | GCT:210 ACC:0 | CDS | − | | | no annotation | LEUM_1424 | |
| 1418878 | TGCCTCG | AGTCTCA | AGTCTCA:149 | CDS | − | | | no annotation | LEUM_1424 | |
| | | | TGCCTCG:0 | | | | | | | |
| 1418950 | ACTC | GCTT | GCTT:163 ACTC:0 | CDS | − | | | no annotation | LEUM_1424 | |
| 1419097 | CCTA | TCTG | TCTG:175 CCTA:0 | CDS | − | | | no annotation | LEUM_1424 | |
| 1419197 | GTGCT | TTGCC | TTGCC:208 GTGCT:0 | CDS | − | | | no annotation | LEUM_1424 | |
| 1419226 | GTTA | ATTG | ATTG:221 GTTA:0 | CDS | − | | | no annotation | LEUM_1424 | |
| 1419311 | TCG | GCC | GCC:230 TCG:0 | CDS | − | | | no annotation | LEUM_1424 | |
| 1419388 | GCTT | ACTG | ACTG:223 GCTT:0 | CDS | − | | | no annotation | LEUM_1424 | |
| 1419438 | TTTTAG | GTTG | GTTG:162 TTTTAG:0 | CDS | − | | | no annotation | LEUM_1424 | |
| 1429917 | TGGCTCC | AGGCACCTT | AGGCACCTTTAGTC | CDS | − | | | no annotation | LEUM_1434 | |
| | TCTATTTG | TAGTCGTTT | GTTTTA:173 | | | | | | | |
| | TCTTT | TA | TGGCTCCTCTATTTG | | | | | | | |
| | | | TCTTT:0 | | | | | | | |
| 1429993 | TGTG | CGTA | CGTA:204 TGTG:0 | CDS | − | | | no annotation | LEUM_1434 | |
| 1430085 | AGAGT | GGAGC | GGAGC:169 | CDS | − | | | no annotation | LEUM_1434 | |
| | | | AGAGT:0 | | | | | | | |
| 1430128 | GTTG | ATTA | ATTA:172 GTTG:0 | CDS | − | | | no annotation | LEUM_1434 | |
| 1430143 | AGACGTG | GGCTGTA | GGCTGTA:153 | CDS | − | | | no annotation | LEUM_1434 | |
| | | | AGACGTG:0 | | | | | | | |
| 1430176 | CTCT | TTCA | TTCA:177 CTCT:0 | CDS | − | | | no annotation | LEUM_1434 | |
| 1430203 | CCCG | TCCA | TCCA:186 CCCG:0 | CDS | − | | | no annotation | LEUM_1434 | |
| 1430314 | AGCTGTG | GGCAGTCA | GGCAGTCACT:192 | CDS | − | | | no annotation | LEUM_1434 | |
| | ACC | CT | AGCTGTGACC:0 | | | | | | | |
| 1430344 | CAAC | TAAG | TAAG:206 CAAC:0 | CDS | − | | | no annotation | LEUM_1434 | |
| 1430374 | TTCG | CTCA | CTCA:216 TTCG:0 | CDS | − | | | no annotation | LEUM_1434 | |
| 1430413 | TAAA | CAAG | CAAG:214 TAAA:0 | CDS | − | | | no annotation | LEUM_1434 | |
| 1430623 | CTCT | TTCA | TTCA:192 CTCT:0 | CDS | − | | | no annotation | LEUM_1435 | |
| 1430785 | AACCAAT | TACAAAACC | TACAAAACCA:159 | CDS | − | | | no annotation | LEUM_1435 | |
| | CCT | A | AACCAATCCT:0 | | | | | | | |
| 1430806 | CAA | TAG | TAG:183 CAA:0 | CDS | − | | | no annotation | LEUM_1435 | |
| 1430942 | TTAGAAT | GTAGGATT | GTAGGATT:180 | CDS | − | | | no annotation | LEUM_1435 | |
| | C | | TTAGAATC:0 | | | | | | | |
| 1431011 | CTTTTT | TCTTTC | TCTTTC:161 | CDS | − | | | no annotation | LEUM_1435 | |
| | | | CTTTTT:0 | | | | | | | |
| 1431073 | CTTA | TTTT | TTTT:160 CTTA:0 | CDS | − | | | no annotation | LEUM_1435 | |
| 1431088 | CAGA | TAGG | TAGG:142 CAGA:0 | CDS | − | | | no annotation | LEUM_1435 | |
| 1431356 | AAC | TAT | TAT:129 AAC:0 | CDS | − | | | no annotation | LEUM_1435 | |
| 1431525 | TTT | CTC | CTC:143 TTT:0 | CDS | − | | | no annotation | LEUM_1436 | |
| 1431755 | CACC | TACT | TACT:154 CACC:0 | CDS | − | | | no annotation | LEUM_1436 | |
| 1431803 | CGTA | TGTG | TGTG:139 CGTA:0 | CDS | − | | | no annotation | LEUM_1436 | |
| 1432287 | GCAAA | ACAAT | ACAAT:162 | | | | | | | |
| | | | GCAAA:0 | | | | | | | |
| 1432326 | AAAC | TACT | TACT:140 AAAC:0 | | | | | | | |
| 1432336 | TAAAA | GAAAG | GAAAG:143 | | | | | | | |
| | | | TAAAA:0 | | | | | | | |
| 1432349 | TATG | CATA | CATA:141 TATG:0 | CDS | − | | | no annotation | LEUM_1437 | |
| 1432378 | CTGA | TTGG | TTGG:207 CTGA:0 | CDS | − | | | no annotation | LEUM_1437 | |
| 1432717 | AAT | CAC | CAC:213 AAT:0 | CDS | − | | | no annotation | LEUM_1437 | |
| 1433379 | CCA | GCG | GCG:209 CCA:0 | CDS | − | | | no annotation | LEUM_1438 | |
| 1433417 | GGACTTA | AGATTTG | AGATTTG:205 | CDS | − | | | no annotation | LEUM_1438 | |
| | | | GGACTTA:0 | | | | | | | |
| 1433441 | CACA | TACG | TACG:222 CACA:0 | CDS | − | | | no annotation | LEUM_1438 | |
| 1433984 | CGTG | TGTA | TGTA:206 CGTG:0 | CDS | − | | | no annotation | LEUM_1438 | |
| 1436006 | AAAG | GAAA | GAAA:254 AAAG:0 | CDS | − | | | no annotation | LEUM_1440 | |
| 1436796 | CAA | TAC | TAC:92 CAA:0 | | | | | | | |
| 1437736 | CAAA | TAAG | TAAG:245 CAAA:0 | CDS | − | | | no annotation | LEUM_1443 | |
| 1437751 | CTTA | TTTG | TTTG:249 CTTA:0 | CDS | − | | | no annotation | LEUM_1443 | |
| 1441725 | CGCTT | TGCTTT | TGCTTT:165 | | | | | | | |
| | | | CGCTT:0 | | | | | | | |
| 1444575 | CAAAAAA | CAAAAAAA | CAAAAAAAACAAAC:127 | | | | | | | |
| | AAAAAAA | ACAAAC | CAAAAAAAAAAAAAC:0 | | | | | | | |
| | C | | | | | | | | | |
| 1447932 | AAAC | GAAT | GAAT:203 AAAC:0 | CDS | − | | | no annotation | LEUM_1454 | |
| 1474016 | TTAAC | CTAAT | CTAAT:171 TTAAC:0 | CDS | − | | | no annotation | LEUM_1480 | |
| 1475011 | TAGT | CAGC | CAGC:175 TAGT:0 | CDS | − | | | no annotation | LEUM_1481 | |
| 1475048 | TGTG | CGTT | CGTT:194 TGTG:0 | CDS | − | | | no annotation | LEUM_1481 | |
| 1475219 | TTGT | CTGC | CTGC:188 TTGT:0 | CDS | − | | | no annotation | LEUM_1481 | |
| 1477474 | TTAAC | CTAAA | CTAAA:148 TTAAC:0 | CDS | − | | | no annotation | LEUM_1481 | |
| 1501570 | AGATC | GCATG | GCATG:145 | CDS | − | | | no annotation | LEUM_1502 | |
| | | | AGATC:0 | | | | | | | |
| 1501590 | ACA | GCG | GCG:140 ACA:0 | CDS | − | | | no annotation | LEUM_1502 | |
| 1510576 | TAAT | CAAA | CAAA:199 TAAT:0 | CDS | − | | | no annotation | LEUM_1513 | |
| 1518189 | AGGC | GGGT | GGGT:152 AGGC:0 | CDS | − | | | no annotation | LEUM_1520 | engB |
| 1519140 | AGCA | GGCT | GGCT:222 AGCA:0 | CDS | − | | | no annotation | LEUM_1521 | cIpX |
| 1519209 | GGAG | AGAT | AGAT:236 GGAG:0 | CDS | − | | | no annotation | LEUM_1521 | cIpX |
| 1527336 | GTCC | ATCT | ATCT:171 GTCC:0 | CDS | − | | | no annotation | LEUM_1529 | |
| 1539200 | GAAA | AAAG | AAAG:234 GAAA:0 | CDS | − | | | no annotation | LEUM_1539 | |
| 1548015 | CAAACT | AGAACA | AGAACA:112 | CDS | + | | | no annotation | LEUM_1546 | |
| | | | CAAACT:0 | | | | | | | |
| 1553910 | AATT | GATA | GATA:154 AATT:0 | CDS | − | | | no annotation | LEUM_1554 | |
| 1563023 | ATAG | TTAA | TTAA:147 ATAG:0 | | | | | | | |
| 1563156 | CCCC | TCCT | TCCT:161 CCCC:0 | CDS | − | | | no annotation | LEUM_1564 | |
| 1563399 | ACCG | GCCC | GCCC:202 ACCG:0 | CDS | − | | | no annotation | LEUM_1564 | |
| 1570912 | GGGA | AGGG | AGGG:201 GGGA:0 | CDS | − | | | no annotation | LEUM_1569 | |
| 1575438 | GCAAA | ACAAG | ACAAG:118 | | | | | | | |
| | | | GCAAA:0 | | | | | | | |
| 1576436 | TTCT | CTCC | CTCC:188 TTCT:0 | CDS | − | | | no annotation | LEUM_1575 | |
| 1576450 | GTATA | ATATC | ATATC:188 GTATA:0 | CDS | − | | | no annotation | LEUM_1575 | |
| 1576582 | CCTC | ACTT | ACTT:201 CCTC:0 | CDS | − | | | no annotation | LEUM_1575 | |
| 1582261 | CACA | GACG | GACG:210 CACA:0 | CDS | − | | | no annotation | LEUM_1578 | |
| 1582441 | TACTGCA | CACCGCG | CACCGCG:178 | CDS | − | | | no annotation | LEUM_1578 | |
| | | | TACTGCA:0 | | | | | | | |
| 1589522 | ACTGC | GCCGT | GCCGT:119 | CDS | − | | | no annotation | LEUM_1586 | |
| | | | ACTGC:0 | | | | | | | |
| 1622472 | TTATAT | ACGTAC | ACGTAC:247 | CDS | − | | | no annotation | LEUM_1624 | |
| | | | TTATAT:0 | | | | | | | |
| 1624045 | AGCCTAC | GCCCGAT | GCCCGAT:111 | CDS | − | | | no annotation | LEUM_1627 | |
| | | | AGCCTAC:0 | | | | | | | |
| 1624058 | CAAG | GAGA | GAGA:110 CAAG:0 | CDS | − | | | no annotation | LEUM_1627 | |
| 1624079 | TATT | AATCA | AATCA:164 TATT:0 | | | | | | | |
| 1624096 | ATTA | GTTG | GTTG:184 ATTA:0 | | | | | | | |
| 1624117 | TAG | CAA | CAA:203 TAG:0 | | | | | | | |
| 1624234 | GCCGCCA | ACCACCG | ACCACCG:231 | CDS | − | | | no annotation | LEUM_1628 | |
| | | | GCCGCCA:0 | | | | | | | |
| 1624336 | TTGA | CTGG | CTGG:149 TTGA:0 | CDS | − | | | no annotation | LEUM_1628 | |
| 1624351 | ATTACCA | GTTCCCG | GTTCCCG:149 | CDS | − | | | no annotation | LEUM_1628 | |
| | | | ATTACCA:0 | | | | | | | |
| 1624431 | TGTTG | AGTTA | AGTTA:98 TGTTG:0 | CDS | − | | | no annotation | LEUM_1628 | |
| 1624459 | CTTA | TTGT | TTGT:84 CTTA:0 | CDS | − | | | no annotation | LEUM_1628 | |
| 1624574 | TTG | GTA | GTA:149 TTG:0 | CDS | − | | | no annotation | LEUM_1628 | |
| 1624609 | GCCG | TCCA | TCCA:180 GCCG:0 | CDS | − | | | no annotation | LEUM_1628 | |
| 1624618 | TCCG | GCCA | GCCA:193 TCCG:0 | CDS | − | | | no annotation | LEUM_1628 | |
| 1624654 | GTTGGAA | ATTTGAG | ATTTGAG:220 | CDS | − | | | no annotation | LEUM_1628 | |
| | | | GTTGGAA:0 | | | | | | | |
| 1624720 | TAA | CAT | CAT:230 TAA:0 | CDS | − | | | no annotation | LEUM_1628 | |
| 1624729 | AGCG | GGCA | GGCA:229 AGCG:0 | CDS | − | | | no annotation | LEUM_1628 | |
| 1624843 | TAG | CAA | CAA:250 TAG:0 | CDS | − | | | no annotation | LEUM_1628 | |
| 1624858 | ATTA | GTTG | GTTG:243 ATTA:0 | CDS | − | | | no annotation | LEUM_1628 | |
| 1624900 | TGCG | AGCA | AGCA:250 TGCG:0 | CDS | − | | | no annotation | LEUM_1628 | |
| 1624918 | GGCTAGC | AGCCAGT | AGCCAGT:239 | CDS | − | | | no annotation | LEUM_1628 | |
| | | | GGCTAGC:0 | | | | | | | |
| 1624978 | CACCGAG | GACTGAA | GACTGAA:222 | CDS | − | | | no annotation | LEUM_1628 | |
| | | | CACCGAG:0 | | | | | | | |
| 1625140 | AAACGAA | GAATGAG | GAATGAG:202 | CDS | − | | | no annotation | LEUM_1628 | |
| | | | AAACGAA:0 | | | | | | | |
| 1625152 | ATAATTTG | GTAGCTTGT | GTAGCTTGT:206 | CDS | − | | | no annotation | LEUM_1628 | |
| | C | | ATAATTTGC:0 | | | | | | | |
| 1625209 | CACG | TACA | TACA:233 CACG:0 | CDS | − | | | no annotation | LEUM_1628 | |
| 1629235 | GATG | TATA | TATA:176 GATG:0 | CDS | − | | | no annotation | LEUM_1635 | |
| 1629250 | ATTA | GTTG | GTTG:180 ATTA:0 | CDS | − | | | no annotation | LEUM_1635 | |
| 1629328 | TGTGTTC | CATATTTAG | CATATTTAGAGAC:159 | CDS | − | | | no annotation | LEUM_1635 | |
| | AAAGAT | AGAC | TGTGTTCAAAGAT:0 | | | | | | | |
| 1629619 | TAATGCG | CAGTGCA | CAGTGCA:203 | CDS | − | | | no annotation | LEUM_1635 | |
| | | | TAATGCG:0 | | | | | | | |
| 1629658 | TATC | GATT | GATT:223 TATC:0 | CDS | − | | | no annotation | LEUM_1635 | |
| 1629722 | ACACCTG | TCTGCTAA | TCTGCTAA:130 | CDS | − | | | no annotation | LEUM_1635 | |
| | | | ACACCTG:0 | | | | | | | |
| 1629759 | ATGA | GTGC | GTGC:191 ATGA:0 | CDS | − | | | no annotation | LEUM_1635 | |
| 1650708 | TAAC | AAAT | AAAT:59 TAAC:0 | CDS | − | | | no annotation | LEUM_1656 | |
| 1650750 | AGGAATC | ATAGATTGG | ATAGATTGGCTCG:35 | | | | | | | |
| | GTTCA | CTCG | AGGAATCGTTCA:0 | | | | | | | |
| 1650948 | ACGCATT | GCGCCTC | GCGCCTC:199 | CDS | − | | | no annotation | LEUM_1657 | |
| | | | ACGCATT:0 | | | | | | | |
| 1651008 | ATTG | GTTA | GTTA:221 ATTG:0 | CDS | − | | | no annotation | LEUM_1657 | |
| 1651041 | TAT | CAC | CAC:223 TAT:0 | CDS | − | | | no annotation | LEUM_1657 | |
| 1651098 | ATA | GTC | GTC:188 ATA:0 | | | | | | | |
| 1651117 | GTGCA | GATA | GATA:133 GTGCA:0 | | | | | | | |
| 1651140 | GCCA | ACCG | ACCG:210 GCCA:0 | | | | | | | |
| 1651201 | TTCC | CTCT | CTCT:224 TTCC:0 | CDS | − | | | no annotation | LEUM_1658 | |
| 1656232 | GCCT | ACCC | ACCC:197 GCCT:0 | CDS | − | | | no annotation | LEUM_1671 | |
| 1661069 | CACT | AACC | AACC:262 CACT:0 | CDS | − | | | no annotation | LEUM_1680 | |
| 1665094 | TTTTAAAC | CTTCAAATC | CTTCAAATCATCG:164 | CDS | + | | | no annotation | LEUM_1690 | |
| | CGTCA | ATCG | TTTTAAACCGTCA:0 | | | | | | | |
| 1665117 | CTTCC | ATTCA | ATTCA:176 CTTCC:0 | CDS | + | | | no annotation | LEUM_1690 | |
| 1665274 | GTACGGC | ATATGGG | ATATGGG:200 | CDS | + | | | no annotation | LEUM_1690 | |
| | | | GTACGGC:0 | | | | | | | |
| 1665286 | CCAC | TCAT | TCAT:208 CCAC:0 | CDS | + | | | no annotation | LEUM_1690 | |
| 1665328 | CGGA | TGGC | TGGC:200 CGGA:0 | CDS | + | | | no annotation | LEUM_1690 | |
| 1665337 | GAAAGAC | AAAGGATG | AAAGGATGCC:196 | CDS | + | | | no annotation | LEUM_1690 | |
| | GCT | CC | GAAAGACGCT:0 | | | | | | | |
| 1665424 | GAAA | AAAG | AAAG:171 GAAA:0 | CDS | + | | | no annotation | LEUM_1690 | |
| 1665436 | GTATG | ATACA | ATACA:144 | CDS | + | | | no annotation | LEUM_1690 | |
| | | | GTATG:0 | | | | | | | |
| 1665448 | CAAGCGC | TAAACGT | TAAACGT:139 | CDS | + | | | no annotation | LEUM_1690 | |
| | | | CAAGCGC:0 | | | | | | | |
| 1665484 | ACCTACC | GCCAACT | GCCAACT:153 | CDS | + | | | no annotation | LEUM_1690 | |
| | | | ACCTACC:0 | | | | | | | |
| 1665529 | TTTA | ATTG | ATTG:168 TTTA:0 | CDS | + | | | no annotation | LEUM_1690 | |
| 1665572 | AGAAC | GGAAT | GGAAT:198 | CDS | + | | | no annotation | LEUM_1690 | |
| | | | AGAAC:0 | | | | | | | |
| 1665664 | GGG | AGA | AGA:206 GGG:0 | CDS | + | | | no annotation | LEUM_1690 | |
| 1665752 | TTACAA | CTGCAG | CTGCAG:201 | CDS | + | | | no annotation | LEUM_1690 | |
| | | | TTACAA:0 | | | | | | | |
| 1665790 | GATTACT | AATAACA | AATAACA:195 | CDS | + | | | no annotation | LEUM_1690 | |
| | | | GATTACT:0 | | | | | | | |
| 1665814 | TAGT | CAGC | CAGC:202 TAGT:0 | CDS | + | | | no annotation | LEUM_1690 | |
| 1666025 | TTAT | ATAC | ATAC:134 TTAT:0 | | | | | | | |
| 1667151 | TAAAAAA | TAAAAAAA | TAAAAAAAG:78 | | | | | | | |
| | T | G | TAAAAAAT:0 | | | | | | | |
| 1669413 | AAACA | GAACG | GAACG:158 | CDS | + | | | no annotation | LEUM_1695 | |
| | | | AAACA:0 | | | | | | | |
| 1670484 | ACCT | TCCC | TCCC:177 ACCT:0 | CDS | − | | | no annotation | LEUM_1696 | |
| 1672983 | ACTGG | GCTGT | GCTGT:189 | CDS | + | | | no annotation | LEUM_1698 | |
| | | | ACTGG:0 | | | | | | | |
| 1684163 | GTCTC | ATCTT | ATCTT:153 GTCTC:0 | | | | | | | |
| 1695377 | ACCG | GCCA | GCCA:273 ACCG:0 | CDS | − | | | no annotation | LEUM_1726 | |
| 1696196 | GGCCGCT | TGCAGCCAA | TGCAGCCAACATA:189 | CDS | − | | | no annotation | LEUM_1726 | |
| | AGCATG | CATA | GGCCGCTAGCATG:0 | | | | | | | |
| 1696244 | TCGCAA | CCGTAG | CCGTAG:215 | CDS | − | | | no annotation | LEUM_1726 | |
| | | | TCGCAA:0 | | | | | | | |
| 1716146 | TAATT | CAATC | CAATC:45 TAATT:0 | | | | | | | |
| 1717930 | ATCA | GTCT | GTCT:47 ATCA:0 | CDS | − | | | no annotation | LEUM_1748 | |
| 1717975 | ATCGATG | GTCTATA | GTCTATA:22 | CDS | − | | | no annotation | LEUM_1748 | |
| | | | ATCGATG:0 | | | | | | | |
| 1718317 | ATCG | GTCT | GTCT:10 ATCG:0 | CDS | − | | | no annotation | LEUM_1748 | |
| 1718353 | ATTT | GTTC | GTTC:22 ATTT:0 | CDS | − | | | no annotation | LEUM_1748 | |
| 1719685 | GGA | AGG | AGG:289 GGA:2 | CDS | − | | | no annotation | LEUM_1748 | |
| 1725927 | TAGCC | CAGCT | CAGCT:186 | CDS | − | | | no annotation | LEUM_1752 | |
| | | | TAGCC:1 | | | | | | | |
| 1726130 | GCTA | TCTG | TCTG:43 GCTA:0 | CDS | − | | | no annotation | LEUM_1752 | |
| 1726179 | TATCC | CAGCT | CAGCT:65 TATCC:0 | CDS | − | | | no annotation | LEUM_1752 | |
| 1726202 | GCTA | TCTG | TCTG:90 GCTA:0 | CDS | − | | | no annotation | LEUM_1752 | |
| 1726215 | TAGCC | CAGCT | CAGCT:95 TAGCC:0 | CDS | − | | | no annotation | LEUM_1752 | |
| 1726251 | CAGCT | TAGCC | TAGCC:143 | CDS | − | | | no annotation | LEUM_1752 | |
| | | | CAGCT:2 | | | | | | | |
| 1756654 | TCTAC | GCTAT | GCTAT:128 TCTAC:0 | | | | | | | |
| 1756824 | ATC | GTA | GTA:145 ATC:0 | CDS | − | | | no annotation | LEUM_1786 | |
| 1757247 | GAAA | AAAG | AAAG:196 GAAA:0 | CDS | − | | | no annotation | LEUM_1786 | |
| 1759552 | TACT | CACC | CACC:256 TACT:0 | CDS | + | | | no annotation | LEUM_1788 | |
| 1759606 | GGCG | AGCA | AGCA:266 GGCG:0 | CDS | + | | | no annotation | LEUM_1788 | |
| 1760925 | ACCCGAT | GCCACTAG | GCCACTAGGCTGCAT:37 | | | | | | | |
| | GGGTTGT | GCTGCAT | ACCCGATGGGTTGTATT:0 | | | | | | | |
| | ATT | | | | | | | | | |
| 1760955 | CAAATGA | TAAGTGG | TAAGTGG:35 | CDS | − | | | no annotation | LEUM_1791 | |
| | | | CAAATGA:0 | | | | | | | |
| 1760994 | GGCTGCA | AGCAGCGA | AGCAGCGAAAGCAG | CDS | − | | | no annotation | LEUM_1791 | |
| | AACGCTG | AAGCAGCG | CGCGTAAACGAAGT:37 | | | | | | | |
| | CACGCAG | CGTAAACG | GGCTGCAAACGCTG | | | | | | | |
| | GCGCAGC | AAGT | CACGCAGGCGCAGC:0 | | | | | | | |
| 1761057 | CTTGGGG | TTTTGGT | TTTTGGT:167 | CDS | − | | | no annotation | LEUM_1791 | |
| | | | CTTGGGG:0 | | | | | | | |
| 1761069 | CTGGGGT | TTGTGGAAT | TTGTGGAATTAATAC | CDS | − | | | no annotation | LEUM_1791 | |
| | ATCAAAA | TAATACTGT | TGTCACT:168 | | | | | | | |
| | CGGTTAC | CACT | CTGGGGTATCAAAA | | | | | | | |
| | A | | CGGTTACA:0 | | | | | | | |
| 1761096 | GTTA | ATTG | ATTG:166 GTTA:0 | CDS | − | | | no annotation | LEUM_1791 | |
| 1761107 | CTGCCTG | TTGCTTGT | TTGCTTGT:173 | CDS | − | | | no annotation | LEUM_1791 | |
| | C | | CTGCCTGC:0 | | | | | | | |
| 1764663 | UC | CTG | CTG:125 TTC:0 | CDS | + | | | no annotation | LEUM_1793 | |
| 1766295 | TAA | CAG | CAG:302 TAA:0 | CDS | − | | | no annotation | LEUM_1794 | |
| 1776537 | CGA | AGC | AGC:191 CGA:0 | CDS | − | | | no annotation | LEUM_1803 | |
| 1790033 | CTGT | TTGC | TTGC:198 CTGT:0 | CDS | − | | | no annotation | LEUM_1817 | |
| 1824412 | CAA | AAG | AAG:178 CAA:0 | CDS | − | | | no annotation | LEUM_1850 | |
| 1830003 | GAGA | AAGG | AAGG:208 GAGA:0 | | | | | | | |
| 1842065 | ACCA | GCCC | GCCC:231 ACCA:0 | CDS | − | | | no annotation | LEUM_1868 | atpC |
| 1857246 | ATTACCTT | GTTATCAAA | GTTATCAAAGGTAA | | | | | | | |
| | TGATAAC | GGTAAT | T:71 | | | | | | | |
| | | | ATTACCTTTGATAAC:0 | | | | | | | |
| 1860337 | AGA | GGG | GGG:145 AGA:0 | CDS | − | | | no annotation | LEUM_1886 | |
| 1861225 | CTTTGCA | TTTTACG | TTTTACG:221 | CDS | − | | | no annotation | LEUM_1888 | |
| | | | CTTTGCA:0 | | | | | | | |
| 1875169 | ATT | GTC | GTC:252 ATT:0 | CDS | − | | | no annotation | LEUM_1900 | |
| 1878574 | ACG | AA | AA:157 ACG:1 | | | | | | | |
| 1878900 | GCAAGT | ATAAGC | ATAAGC:121 | CDS | + | | | no annotation | LEUM_1905 | |
| | | | GCAAGT:0 | | | | | | | |
| 1878918 | GTG | TTT | TTT:121 GTG:0 | CDS | + | | | no annotation | LEUM_1905 | |
| 1878926 | CTTT | TTTC | TTTC:114 CTTT:0 | CDS | + | | | no annotation | LEUM_1905 | |
| 1878938 | ATAGA | GTAA | GTAA:113 ATAGA:0 | CDS | + | | | no annotation | LEUM_1905 | |
| 1878945 | TCCC | GACG | GACG:112 TCCC:0 | | | | | | | |
| 1878959 | GTAT | TTAA | TTAA:139 GTAT:0 | | | | | | | |
| 1879309 | CCTAGCC | TCTGGCCT | TCTGGCCT:176 | | | | | | | |
| | A | | CCTAGCCA:0 | | | | | | | |
| 1882947 | AGTAGT | GGTTGC | GGTTGC:244 | | | | | | | |
| | | | AGTAGT:0 | | | | | | | |
| 1882969 | TACAT | GACAC | GACAC:243 | | | | | | | |
| | | | TACAT:0 | | | | | | | |
| 1886783 | CCAATCA | TCGATCG | TCGATCG:207 | CDS | + | | | no annotation | LEUM_1917 | |
| | | | CCAATCA:0 | | | | | | | |
| 1887546 | TAGG | CAAA | CAAA:137 TAGG:0 | CDS | − | | | no annotation | LEUM_1919 | |
| 1887555 | ACGTGTT | TCGCGTA | TCGCGTA:147 | CDS | − | | | no annotation | LEUM_1919 | |
| | | | ACGTGTT:0 | | | | | | | |
| 1887567 | CAATGAA | TAGAGAGC | TAGAGAGCCA:147 | CDS | − | | | no annotation | LEUM_1919 | |
| | CCG | CA | CAATGAACCG:0 | | | | | | | |
| 1887582 | TTCA | CTCG | CTCG:153 TTCA:0 | CDS | − | | | no annotation | LEUM_1919 | |
| 1887645 | GGCT | AGCC | AGCC:249 GGCT:0 | CDS | − | | | no annotation | LEUM_1919 | |
| 1887654 | CTTG | TTTA | TTTA:252 CTTG:0 | CDS | − | | | no annotation | LEUM_1919 | |
| 1887666 | ACGAAGC | GCGCAAT | GCGCAAT:172 | CDS | − | | | no annotation | LEUM_1919 | |
| | | | ACGAAGC:0 | | | | | | | |
| 1887684 | CTGG | TTGT | TTGT:196 CTGG:0 | CDS | − | | | no annotation | LEUM_1919 | |
| 1887711 | TGTCACTT | AGTTACCTG | AGTTACCTGG:239 | CDS | − | | | no annotation | LEUM_1919 | |
| | GA | G | TGTCACTTGA:0 | | | | | | | |
| 1887732 | GCCG | ACCA | ACCA:275 GCCG:0 | CDS | − | | | no annotation | LEUM_1919 | |
| 1887771 | CTTC | TTTT | TTTT:299 CTTC:0 | CDS | − | | | no annotation | LEUM_1919 | |
| 1887795 | CGCTCCA | TGCACCG | TGCACCG:316 | CDS | − | | | no annotation | LEUM_1919 | |
| | | | CGCTCCA:0 | | | | | | | |
| 1887821 | ATTTA | GCTTG | GCTTG:277 ATTTA:0 | CDS | − | | | no annotation | LEUM_1919 | |
| 1887831 | GTTTCCA | ATTACCG | ATTACCG:281 | CDS | − | | | no annotation | LEUM_1919 | |
| | | | GTTTCCA:0 | | | | | | | |
| 1887852 | GTGA | ATGT | ATGT:312 GTGA:0 | CDS | − | | | no annotation | LEUM_1919 | |
| 1887867 | ACTG | GCTA | GCTA:324 ACTG:0 | CDS | − | | | no annotation | LEUM_1919 | |
| 1887897 | TAG | CAA | CAA:307 TAG:0 | CDS | − | | | no annotation | LEUM_1919 | |
| 1887906 | AGCA | GGCG | GGCG:305 AGCA:0 | CDS | − | | | no annotation | LEUM_1919 | |
| 1896684 | TCAGC | CCAGA | CCAGA:220 | CDS | − | | | no annotation | LEUM_1927 | |
| | | | TCAGC:0 | | | | | | | |
| 1897538 | GCGC | ACGT | ACGT:286 GCGC:0 | CDS | − | | | no annotation | LEUM_1928 | |
| 1915818 | AGTT | GGTC | GGTC:305 AGTT:0 | CDS | − | | | no annotation | LEUM_1944 | |
| 1917475 | TTA | CTC | CTC:134 TTA:0 | CDS | − | | | no annotation | LEUM_1945 | |
| 1933246 | TCA | CCG | CCG:225 TCA:0 | CDS | + | | | no annotation | LEUM_1960 | |
| 1933618 | CATT | TATA | TATA:200 CATT:0 | CDS | + | | | no annotation | LEUM_1960 | |
| 1933723 | GCCCA | TCCCG | TCCCG:175 | CDS | + | | | no annotation | LEUM_1960 | |
| | | | GCCCA:0 | | | | | | | |
| 1933941 | GTCT | ATT | ATT:134 GTCT:0 | | | | | | | |
| 1934018 | ATATTAC | TTGTTAT | TTGTTAT:133 | | | | | | | |
| | | | ATATTAC:0 | | | | | | | |
| 1934029 | ACAA | GTAT | GTAT:135 ACAA:0 | | | | | | | |
| 1934072 | GTAA | ATA | ATA:142 GTAA:0 | | | | | | | |
| 1934080 | ATGTGGC | GTGTTGT | GTGTTGT:142 | | | | | | | |
| | | | ATGTGGC:0 | | | | | | | |
| 1952692 | GAATA | TAATG | TAATG:97 GAATA:0 | | | | | | | |
| 1952721 | GAAG | AAAT | AAAT:82 GAAG:0 | | | | | | | |
| 1952732 | GTGTT | TCGTC | TCGTC:78 GTGTT:0 | | | | | | | |
| 1953810 | CGGTG | TTGTA | TTGTA:462 | | | | | | | |
| | | | CGGTG:0 | | | | | | | |
| 1960043 | CAATT | TAATC | TAATC:36 CAATT:0 | | | | | | | |
| 1960073 | TTTGGG | AAGGGA | AAGGGA:39 | | | | | | | |
| | | | TTTGGG:0 | | | | | | | |
| 1960134 | TGTGTTA | AGTGCTATA | AGTGCTATATTT:34 | CDS | − | | | no annotation | LEUM_1991 | |
| | AATAC | TTT | TGTGTTAAATAC:0 | | | | | | | |
| 1960163 | GTCA | ATCT | ATCT:36 GTCA:0 | CDS | − | | | no annotation | LEUM_1991 | |
| 1960179 | ATTGC | CTTAA | CTTAA:39 ATTGC:0 | CDS | − | | | no annotation | LEUM_1991 | |
| 1960376 | TGCT | AGCA | AGCA:107 TGCT:0 | CDS | − | | | no annotation | LEUM_1991 | |
| 1960390 | GTCTT | ACCTC | ACCTC:106 GTCTT:0 | CDS | − | | | no annotation | LEUM_1991 | |
| 1960567 | AAA | CAC | CAC:136 AAA:0 | | | | | | | |
| 1960585 | CTGCA | TTGCG | TTGCG:122 | | | | | | | |
| | | | CTGCA:0 | | | | | | | |
| 1960664 | TGTC | CGTT | CGTT:161 TGTC:0 | | | | | | | |
| 1969902 | GTC | ATT | ATT:182 GTC:0 | CDS | + | | | no annotation | LEUM_2001 | |
| 1969941 | GTTTA | ATTTT | ATTTT:173 GTTTA:0 | CDS | + | | | no annotation | LEUM_2001 | |
| 1970013 | TTAT | CTGC | CTGC:152 TTAT:0 | CDS | + | | | no annotation | LEUM_2001 | |
| 1978224 | AGTAT | GGTAC | GGTAC:277 | CDS | − | | | no annotation | LEUM_2010 | |
| | | | AGTAT:0 | | | | | | | |
| 1980589 | CTTGT | TTTGC | TTTGC:192 CTTGT:0 | | | | | | | |
| 1994040 | TAATT | GAATC | GAATC:291 TAATT:0 | CDS | − | | | no annotation | LEUM_2027 | |
| 1996966 | GTGG | ATGA | ATGA:363 GTGG:0 | CDS | − | | | no annotation | LEUM_2030 | |
| 1996984 | GATT | AATC | AATC:258 GATT:0 | CDS | − | | | no annotation | LEUM_2030 | |
| 1996993 | GGCAGGC | AGCTGGT | AGCTGGT:241 | CDS | − | | | no annotation | LEUM_2030 | |
| | | | GGCAGGC:0 | | | | | | | |
| 1997007 | GACCCCG | ATCCTCGCT | ATCCTCGCTCCGGT:235 | CDS | − | | | no annotation | LEUM_2030 | |
| | TTCAGGC | CCGGT | GACCCCGTTCAGGC:0 | | | | | | | |
| 1997032 | CACA | AACG | AACG:318 CACA:0 | CDS | − | | | no annotation | LEUM_2030 | |
| 2025691 | GCTA | ACTG | ACTG:240 GCTA:0 | CDS | − | | | no annotation | LEUM_2060 | |
| 2025829 | AACA | GACG | GACG:213 AACA:0 | CDS | − | | | no annotation | LEUM_2060 | |
| 2026633 | GCAG | ACAA | ACAA:327 GCAG:0 | CDS | − | | | no annotation | LEUM_2061 | |
| 2036598 | GCCT | ACCC | ACCC:291 GCCT:0 | CDS | − | | | no annotation | LEUM_2072 | |
| 2037136 | TCGA | CCGT | CCGT:198 TCGA:0 | | | | | | | |
| 2037152 | TAACA | GAACG | GAACG:210 | | | | | | | |
| | | | TAACA:0 | | | | | | | |
| 2037383 | TCCA | CCCT | CCCT:285 TCCA:0 | CDS | − | | | no annotation | LEUM_2073 | |
| 2037417 | CGT | TGC | TGC:259 CGT:0 | CDS | − | | | no annotation | LEUM_2073 | |
| 2037438 | GTATC | TTATT | TTATT:286 GTATC:0 | CDS | − | | | no annotation | LEUM_2073 | |

### Fermentation

Preparation of starter cultures: Pooled cultures of Leuconostoc mesenteroides (BF1, BF2) and Lactobacillus plantarum (B1, B2, B3, B4, B5) isolated from broccoli (described in fermentation patent and deposited) were used for fermentation. The lactic acid bacteria stock cultures, which were stored at −80° C., were activated by inoculation into 10 mL MRS broth (Oxoid, Victoria, Australia) and incubation at 30° C. for 24 hours to get the primary inoculum. 2 mL of the primary cultures were inoculated into 200 mL of MRS broth to obtain the secondary cultures. After 24 h incubation, the 6 secondary cultures were centrifuged, washed twice with sterile phosphate buffer saline (PBS) and. each of the culture was resuspended in Milli-Q water at a concentration of 10 log colony-forming units per millilitre (CFU/mL) to obtain an initial biomass of 8 log CFU/mL in 100 gm broccoli puree samples. The L. plantarum cultures were mixed with the Leu. mesenteroides cultures at 1:1 proportion prior to inoculation into the broccoli puree samples. Broccoli puree samples were inoculated with the cultures. Each broccoli puree sample was inoculated with the prepared starter culture at an initial level of 8 log CFU/g. The fermentation experiment was carried out at 30° C. until the pH reached ˜4.0.

### Methods

The raw and processed broccoli's were assessed for their potential to improve gut health by examining their ability to influence gut microbial activity and stimulate the production of beneficial short chain fatty acids (SCFA) using an in vitro model. That is, broccoli samples of interest were added to tubes containing human stool (from healthy donors collected as described in Charoensiddhi et al. (2016) diluted in media and subsequently incubated to allow fermentation under anaerobic conditions for 24 hours. The broccoli products tested were added to the fermentation medium at 1.5% w/v. The ferment mixtures were then analysed for levels of SCFA, especially the main forms acetic acid, propionic acid and butyric acid, and also analysed using Q-PCR and sequencing methods to ascertain any changes in the microbial populations in response to the different broccoli substrates. The method comprises converting salts and esters of short chain fatty acids to short chain fatty acids and measuring acetic acid, propionic acid and butyric acid. Thus, the levels assessed are indicative of an increase in: acetic acid and acetate; propionic acid and propionate: and butyric acid and butyrate. The in vitro fermentation method, SCFA analysis and Q-PCR methods (for the targeted measure of changes in Lactobacillus, Bifidobacterium, E. coli and total bacteria) are as described in Charoensiddhi et al. (2016). A broad assessment of microbial population changes was carried by PCR amplification of the 16S rRNA region of DNA extracted from the ferment samples and the sequencing of the amplified DNA.

### Results

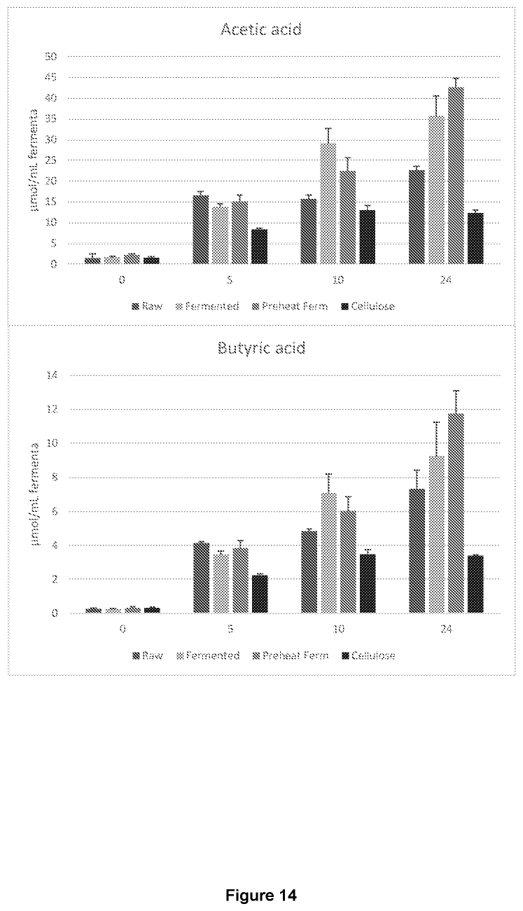

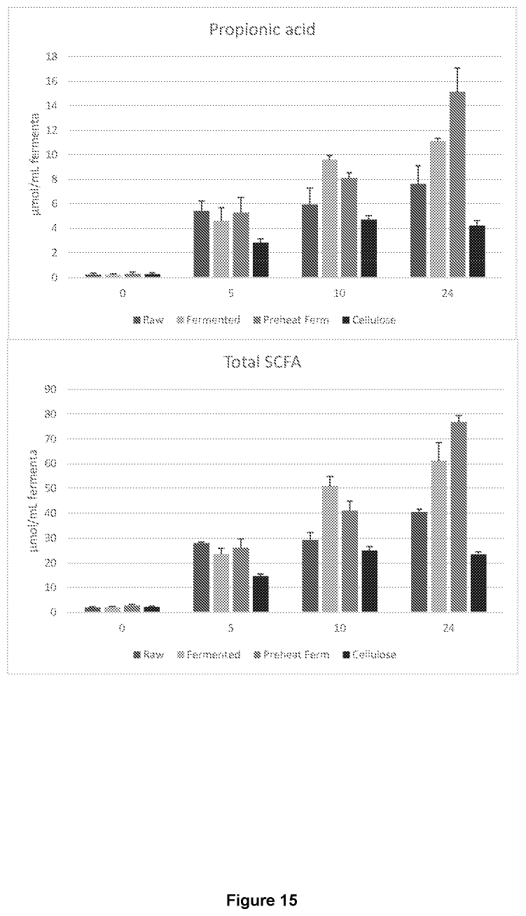

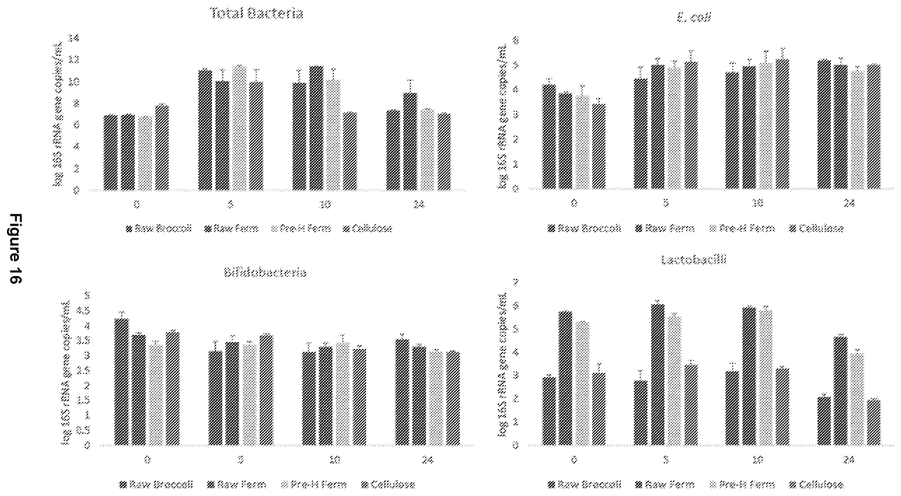

As shown in FIGS. 14 and 15 fermented broccoli and fermented pre-treated broccoli increased short chain fatty acid production 10 and 24 hours after addition compared to unfermented broccoli control and a cellulose control. As shown in FIG. 16 fermented broccoli and fermented pre-treated broccoli has an increased number of lactic acid bacteria (Lactobacillus) compared to the unfermented broccoli control and the cellulose control.

### Example 18—Fermented Broccoli as Delivery Vehicle for Omega-3 Fatty Acids

### Methods

### Sample Preparation

Fresh broccoli (cv. Solitair) was obtained from a local farm (FreshSelect, Werribee South). Following washing, the broccoli florets were cut at approximately 2 cm from the head and were divided into two lots. The first lot was steamed in a steam oven pre-heated to 100° C. (Rational combi oven) to a core temperature of −65° C. and held at that temperature for 3 min to inactivate the protein co-factor Epithispecifier protein (ESP) followed by cooling in ice-water. Following cooling, the broccoli florets were mixed with water (3 parts broccoli and 2 parts water) and were pureed for 1 min using a kitchen blender (Nutribullet pro 900 series, LLC, USA). The second lot was similarly processed into puree without preheating. Both the control and the preheated broccoli puree were further divided into two lots; fish oil was added at 50% loading to one lot from each group. The fish oil was added at 6% (w/w) since the total solid contents of the broccoli puree samples were ˜6%. Following the addition of oil, the mixture was homogenised into a coarse emulsion using a laboratory scale mixer (Silverson, Model: L4R, USA) at a stirring speed ranging from 2500 rpm to 6000 rpm over 5 minutes. Samples from each lot were further divided into two; one for use as control and the second for subsequent fermentation. The control and the preheated-control samples with or without added oil were immediately frozen after preparation for subsequent freeze drying. The samples and their designation are provided in Table 20.

TABLE 20
Processed broccoli samples and their designation.

| Sample type | Sample designation |
|---|---|
| Control broccoli | C-NF |
| Control broccoli with tuna oil | C-To-NF |
| Control fermented broccoli | C-F |
| Control broccoli fermented with tuna oil | C-To-F |
| Preheated broccoli | Ph-NF |
| Preheated broccoli with tuna oil | Ph-To-NF |
| Preheated fermented broccoli | Ph-F |
| Preheated broccoli fermented with tuna | Ph-To-F |
| oil | |

### Preparation of Starter Culture for Fermentation

A cocktail of seven lactic acid bacteria strains isolated from broccoli i.e. 5 Lactobacillus plantarem strains (B1, B2, B3, B4, B5) and two Leuconostoc mesenteroides (BF1, BF2) were used as a starter for the fermentation of broccoli puree samples. To obtain the primary inoculum, lactic acid bacteria cultures which were stored at −80° C. were inoculated into 10 mL of MRS broth (Oxoid, Victoria, Australia) and incubated at 30° C. for 18 hrs. This was followed by a secondary culture where 2 mL of the primary culture was inoculated into 200 mL of MRS broth and incubated for 18 hrs at 30° C. The cultures were collected by centrifugation at 3500 g for 15 min at 4° C., washed twice with sterile phosphate buffer saline (PBS), and were suspended in Milli-Q water at a concentration of 10 log CFU/mL. Then, all the L. plantarum and Leu. mesenteroides cultures were mixed together, glycerol was added to the mixture, the pooled culture was stored at −80° C. until use as a starter culture for broccoli puree fermentation. Prior to the fermentation experiment, the cultures were thawed, washed twice with PBS and resuspended in Milli-Q water.

### Fermentation Experiments

Each broccoli puree and emulsion sample (˜450 g) was inoculated with the prepared starter culture at a dose of 8 log CFU/g. The fermentation experiment was carried out at 30° C. until the pH reached ˜4.0, which was from 15 hrs to 48 hours of incubation depending on the sample. Once the fermentation was completed, samples (labelled as day 0 samples) were taken for microbial, physicochemical and chemical analyses. The rest of the ferments were packed in aluminium foil bags and were flat frozen (˜2 cm thick) and stored at −20° C. until freeze drying. The samples were freeze dried using Cuddon freeze dryer (New Zealand) at 25° C. and 2.5 mbar vacuum within 2 days. The freeze-dried powders were used in subsequent analyses and storage stability studies. Following freeze drying, samples were aliquoted for the storage stability trial as described below and the rest of the samples were kept at −80° C. until microbial and chemical analyses. All experiments were conducted in duplicates.

### Storage Stability Study

For the storage stability study, 1 g powder samples were aliquoted into amber glass vials, flushed with nitrogen and tightly capped and stored at 25° C. Samples were taken every 10 days for FAME analysis.

### Microbial Analysis

The microbiological analyses of the samples were conducted following standard methods in literature (Cai et al., 2019). Accordingly, total lactic acid bacteria (LAB) was enumerated by plating on De Man, Rogosa and Sharpe agar (MRS), total Enterobacteriaceae on VRBGA (Violet Red Blue Glucose Agar), and yeasts and mould on PDA (Potato Dextrose Agar) agar plates with the pH adjusted to 3.5 using 10% tartaric acid. For each sample, the broccoli suspension was serially diluted with sterilized peptone saline diluent and 0.1 mL of the diluted samples were plated onto the agar plates in duplicate. After aerobic incubation at 25° C. for 72 h (PDA), 37° C. for 24 h (VRBGA), and anaerobic incubation at 37° C. for 72 h (MRS), respectively, the colony forming units (CFU) were counted.

### Fatty Acid Methyl Ester (FAME) Analysis

The oxidative stability of the fish oil encapsulated in freeze-dried broccoli powders (fermented/unfermented broccoli) was evaluated after freeze drying and during storage at 25° C. by direct methylation of microencapsulated powder. The content of Eicosapentaenoic acid (EPA, C20:5 ω3) and Docosahexaenoic acid (DHA, C22:6 ω3) (mg per g powder) was calculated from the FAME data. The remaining EPA & DHA content (%) during the storage time for each powder was also analysed. Each sample was analysed in triplicate. Fatty acid composition was determined by gas chromatography (GC). A direct methylation of the powder for fatty acid analysis was conducted in accordance with a previously reported method (Zhou et al., 2009).

Fatty acid methyl esterification and extraction was conducted as follows. A mixture of the powder (10 f 0.01 mg) with the 75 μl internal standards (0.75 mg of 17:0 Triheptadecanoin, TAG in tolune) were suspended in 0.9 mL 1N methanolic HCl and 0.1 mL of Dichloromethane in argon-flushed 2 mL GC vial. The mixture was subsequently incubated in a shaker water bath (100 rpm) at 80° C. for 2 hr. FAME were extracted with 0.3 mL hexane. Transesterified fatty acids were added with 0.3 ml hexane for GC analysis (Shen et al., 2014).

The samples were quantified by GC following previously described method with some modifications (Shen et al., 2014). FAME solution (1 μL) was injected at a split ratio of 1:40 into a GC column (BPX 70 fused silica column, 30 m, 0.25 mm id and 0.25 lm films, SGE, Australia), installed in a model 7890A GC system equipped with a model 7693 autosampler (Agilent Technologies Australia Pty Ltd., Mulgrave, Victoria 3170, Australia). The GC column temperature was increased from 60 to 170° C. at a rate of 20° C./min, then to 192° C. at a rate of 1° C./min and finally to 220° C. at 20° C./min. The injector and detector (FID) were held at 220 and 250° C., respectively. Agilent Chemstation software [B.04.02 SP2 (256)] was used to integrate GC peak areas. Individual polyunsaturated fatty acids (i.e. Eicosapentaenoic acid, EPA, C20:5 w3 and Docosahexaenoic acid, DHA, C22:6 w3 in the powder (mg fatty acid/g dry weight) were calculated as described in the AOCS official method (AOCS, Method Ce 1b-892009).

### Oxipres Analysis

Oxipres analysis of the oil powder samples was conducted in order to determine the oxidative stability of the fish oil encapsulated in the broccoli matrix under accelerated conditions. It involves exposing the samples to oxygen at high temperature and analysing the rate of oxygen consumption as a measure of oxidation. The induction point for oxidation (IP) is used as a measure of the oxidation stability of the oil powder and is evaluated as the time point in which an inflection is observed in the oxygen pressure versus time plot. The Oxipres IP analysis was conducted using ML OXIPRES (Mikrolab Aarhus A/S, Hojbjerg, Denmark). About 8 grams of samples (containing about 4 g of oil) were used in the analysis. The analysis was conducted at 80° C. and 5 bar oxygen pressure.

### Results

Oxidative Stability of Omega-3 Fatty Acids in Freeze Dried Non-Fermented-Oil Powder and Broccoli Fermented with Oil Powder as Measured by Oxipres

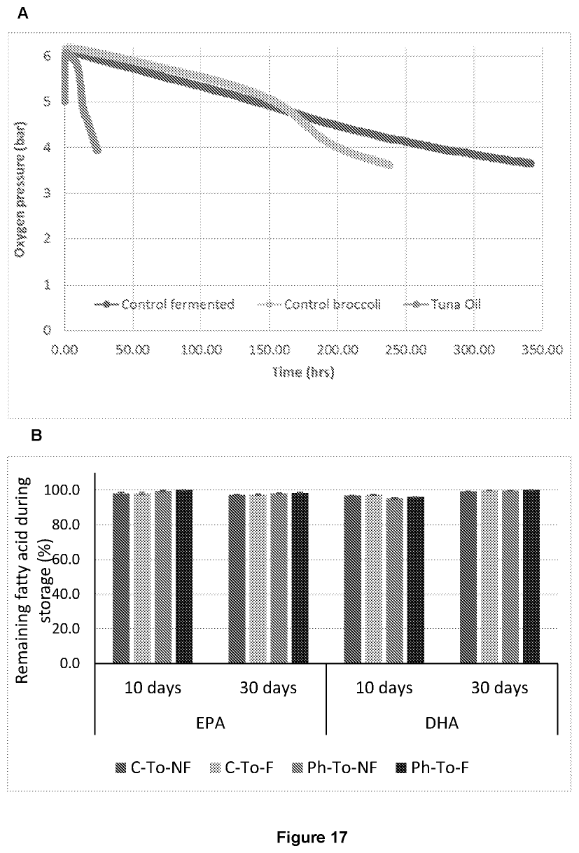

Broccoli-oil, preheated broccoli-oil, broccoli fermented with oil and preheated broccoli fermented with oil had substantially lower oxygen absorption rates compared to the neat oil, indicating that encapsulation of omega-3 fatty acids with all broccoli-based matrices improve the stability of omega-3 fatty acids. Example data comparing the Oxipres trace of neat tuna oil, and broccoli-tuna oil powder and broccoli fermented with oil powder are given in FIG. 17A. The IP of this batch of neat tuna oil was less ˜10 hrs, whereas the IP of the broccoli encapsulated tuna oil powder was ˜158 hrs. No clear IP was observed for the broccoli fermented with oil powder and preheated broccoli fermented with oil powder for up to 350 hrs indicating that addition of oil priot to fermentation further enhances the stabilisation effects on omega-3 fatty acids.

Stability of EPA and DHA in Broccoli Fermented with Oil and Non Fermented Broccoli-Oil Powders During Storage at 25° C.

The levels of omega-3 fatty acids in the different broccoli powders were evaluated during one month storage at 25° C. for samples stored in amber bottles flushed with nitrogen. The levels of both eicosapentaenoic acid (EPA) and docosahexaenoic acid (DHA) remained stable during storage of the broccoli powders (FIG. 17B), indicating that both non-fermented broccoli and broccoli fermented with oil can be used for delivery of omega-3 fatty acids.

### Impact of Tuna Oil on Growth of Lactic Acid Bacteria During Fermentation of Broccoli Samples and Survival During Freeze Drying

The addition of tuna oil into the non-preheated and pre-heated broccoli puree did not inhibit the growth of lactic acid bacteria and the fermentation process. It was observed that the lactic acid bacteria count in the oil samples was slightly higher in both control and preheated samples (Table 21). However, the difference was not statistically significant. There was on average 2.51, 1.68, 1.84 and 2.25 log reduction in lactic acid bacteria count after freeze drying in the control fermented, control fermented with oil, preheated fermented and preheated fermented with oil samples, respectively. The presence of tuna oil improved the survival of lactic acid bacteria in the control fermented samples.

### Example 19—Fermented Broccoli as a Delivery System for Probiotic Bacteria

### Methods

### Sample Preparation

Farm fresh broccoli was sourced from a local farm (Fresh Select). Untreated control (C-NF) and preheated (Ph-NF) broccoli puree samples were prepared as described in Example 18. Fermentation of the control and the preheated puree samples were conducted as described in Example 18. The final pH of the control fermented and the preheated fermented broccoli were 3.93 and 3.72, respectively. Following fermentation, Bifidobacterium animalis subsp. Lactis powdered culture (Chrstian Hansen), as a model probiotic microorganism, was added into the fermented purees (C-F and Ph-F) at 10% dry weight basis. The samples were thoroughly mixed and frozen and freeze dried as described in Example 18. All experiments were conducted in duplicates.

TABLE 21
Lactic acid bacteria count in fermented broccoli samples with and
without added tuna oil (log CFU/g) dry weight basis.

| | Fermented puree | Freeze dried |
|---|---|---|
| | (log CFU/g dry | fermented powder |
| Sample | weight) | (log CFU/g dry weight) |
| Fermented control | 8.6 ± 0.17 | 6.09 ± 0.12 |
| broccoli (C-F) | | |
| Control broccoli | 8.72 ± 0.14 | 7.05 ± 0.33 |
| fermented with tuna oil | | |
| (C-To-F) | | |
| Preheated fermented | 10.84 ± 0.90 | 9.09 ± 0.13 |
| broccoli (Ph-F) | | |
| Preheated broccoli | 11.33 ± 0.39 | 9.0 ± 0.0 |
| fermented with tuna oil | | |
| (Ph-To-F) | | |

### Microbial Analysis

Freeze-dried probiotic powders were rehydrated by dispersing in Buffered peptone water (BPT) in a shaking water bath (3TC, 100 rpm, 1 h). The rehydrated samples were then diluted with Maximum recovery diluent (MRD). De Man, Rogosa and Sharpe agar (MRS agar, Oxoid Ltd, UK) was used for enumeration LAB from the samples as described in Example 18. RCA (reinforced clostridial agar, pH 6.8, Oxoid Ltd, UK) agar was used for enumeration of Bifidobacterium lactis in the samples. The inoculated plates were incubated under anaerobic conditions at 3TC for 48 h. Each viable unit (cells) grown as a colony on the plates was counted as a colony forming unit (CFU) and calculated for the number of CFU per gram of the powder (CFU/g). The viable counts were transformed into log 10 value and loss of the count in the powders were calculated and compared with the control probiotic powders as well as with the value of each powder.

### Simulated In Vitro Digestion

Broccoli powders (C-F-Bifido, and Ph-F-bifido powders) were sequentially exposed to simulated gastric fluid (SGF) and simulated intestinal fluids (SIF). The SGF solution (pH adjusted to 1.2 with HCl) was comprised of sodium chloride (2 g) and pepsin (1.6 g) made up to 1000 mL with Milli-Q water. The SIF (pH adjusted to 6.8 using NaOH) contained anhydrous potassium dihydrogen phosphate (17 g) and pancreatin (3.15 g) made up to 1000 mL with Milli-Q water. For sequential exposure to (SGF+SIF), the freeze-dried powders (0.2 g in 9.8 g of deionized water) was added to SGF solution (12.5 mL) and incubated in a shaking water bath (37° C. for 2 h). The SGF-digested sample was adjusted to pH 6.8 with 1M NaOH, combined with 10 mL of SIF solution and incubated for 20 min prior to addition of CaCl₂ (0.05 M, 2.5 mL) and incubation for a further 2 h and 40 min. The survival of the lactic acid bacteria and the added bifido bacteria cells with/without broccoli matrices were evaluated in the simulated digestive fluids (SGF, and SIF) by plating in the respective media as described above in the microbial analysis section. For sequential exposure to simulated SGF and SIF with bile extract, similar procedure of incubation with 10 mL of SIF solution (with added bile extract (6.25±0.01 g/L) to the SIF solution) was applied to the SGF-digested samples, following which the viability of the lactic acid bacteria and the Bifidobacterium were assessed.

### Results

Survival of Bifidobacterium animalis Subsp. Lactis in Fermented Broccoli and Preheated Fermented Broccoli Matrices During Freeze Drying

The initial viable counts of the samples and the viable counts after the powders production are given in Table 22. Before freeze drying, the fermented broccoli with Bifido powders had 2.60E+10 CFU/g powder. After freeze-drying, the viable count of cells in the fermented broccoli-bifido (C-F-Bifido) powder was 1.65E+09 CFU/g powder and that of the preheated fermented bifido (Ph-F-Bifido) powder was 2.88E+10 CFU/g powder. This represents a loss of 1.2 log CFU/g during freeze-drying process for the control fermented sample whereas no loss was observed in the case of the preheated fermented sample.

TABLE 22
Changes in the viable count of Bifidobacterium animalis
subsp. lactis after freeze-drying of broccoli powders.

| | Viable LAB count/g powder | | |
|---|---|---|---|
| | (log₁₀ transformed value) | | Loss of LAB |
| | Before | After | (log₁₀ transformed |
| | freeze-drying | freeze-drying | value) |
| C-F-Bifido | 10.41 ± 0.10 | 9.22 ± 0.74 | 1.19 |
| Ph-F-Bifido | 10.41 ± 0.10 | 10.46 ± 0.23 | No loss |

Survival of Bifidobacterium animalis Subsp. Lactis in Fermented and Preheated Fermented Broccoli Matrices Following In Vitro Digestion

The survival of Bifidobacterium animalis subsp. Lactis following simulated in-vitro digestion of the fermented and unfermented samples were evaluated. The viable counts prior to and after in vitro digestion (SGF+SIF) are presented in Table 23. The broccoli matrices protected the probiotic bacteria against inactivation during the simulated digestion. The control fermented broccoli provided the best protection with the least viability loss whereas the preheated fermented broccoli provided the least protection for the probiotic organism.

TABLE 23
Survival of Bifidobacterium animalis subsp. Lactis cells as is and in
fermented broccoli matrices after sequential exposure to simulated
gastric fluid (SGF) for 2 h and simulated intestinal fluid (SIF) for 3 h
(without added bile).

| | | | Loss of LAB |
|---|---|---|---|
| | | | compared to |
| | | | Freeze-dried |
| | Viable count/g powder | | powder |
| | (log₁₀ transformed value) | | (log10 |
| | Before in-vitro | After in-vitro | transformed |
| Sample | digestion | digestion | value) |
| Bifido control | 10.41 ± 0.10 | 4.55 ± 0.21 | 5.86 |
| C-F-Bifido | 9.22 ± 0.74 | 5.83 ± 0.29 | 3.39 |
| Ph-F-Bifido | 10.46 ± 0.23 | 5.08 ± 0.14 | 5.39 |

The survival of Bifidobacterium animalis subsp. Lactis after simulated in-vitro digestion of the fermented samples with added bile were also evaluated. The data are presented in Table 24. Overall, higher loss of viability was observed in this case compared to in vitro digestion without added bile. Higher survival was observed in the fermented samples with the control fermented broccoli providing the best protection compared to the bifido control. The preheated fermented samples was less effective than the control fermented sample perhaps due to its lower pH (pH 3.72 compared to pH 3.93 of the control fermented product). The result indicates that fermented broccoli can be used for protected delivery of probiotic microorganisms into the gastrointestinal tract.

### Survival of Lactic Acid Bacteria (Autochthonous and Starter Culture in the Fermented Samples) Following In Vitro Digestion

The survival of lactic acid bacteria in fermented broccoli matrices during simulated in vitro digestion with and without bile were evaluated. Data are presented in Table 25 and 26. The lactic acid bacteria in the broccoli samples survived simulated in vitro digestion with and without bile, indicating that the products can be used for delivering beneficial lactic acid bacteria into the gut for probiotic benefit. Lower survival of lactic acid bacteria was observed in the case of the pre-heated fermented sample compared to the control fermented sample perhaps due to its lower pH compared to all the other non-fermented and fermented samples in this study.

TABLE 24
Survival of Bifidobacterium animalis subsp. Lactis cells as is and in
fermented broccoli matrices after sequential exposure to simulated
gastric fluid (SGF) for 2 h and simulated intestinal fluid (SIF) with
added bile for 3 h.

| | | | Loss of LAB |
|---|---|---|---|
| | | | compared to |
| | | | Freeze-dried |
| | Viable count/g powder | | powder |
| | (log₁₀ transformed value) | | (log10 |
| | Before in-vitro | After in-vitro | transformed |
| Sample | digestion | digestion | value) |
| Bifido control | 10.41 ± 0.10 | 4.24 ± 0.09 | 6.17 |
| C-F-Bifido | 9.22 ± 0.74 | 4.42 ± 0.09 | 4.80 |
| Ph-F-Bifido | 10.46 ± 0.23 | 4.78 ± 0.18 | 5.68 |

TABLE 25
Survival of total lactic acid bacteria (autochthonous and starter
culture in fermented samples) after in-vitro digestion without bile.

| | | | Loss of LAB |
|---|---|---|---|
| | | | compared to |
| | | | Freeze-dried |
| | Viable LAB count/g powder | | powder |
| | (log₁₀ transformed value) | | (log10 |
| | Before in-vitro | Before in-vitro | transformed |
| | digestion | digestion | value) |
| C-F-Bifido | 6.20 ± 0.28 | 5.19 ± 0.21 | 1.01 |
| Ph-F-Bifido | 8.53 ± 0.06 | 5.05 ± 0.11 | 3.48 |

TABLE 26
Survival of total lactic acid bacteria (autochthonous and starter
culture in fermented samples) after in-vitro digestion with bile.

| | | | Loss of LAB |
|---|---|---|---|
| | | | compared to |
| | | | Freeze-dried |
| | Viable LAB count/g powder | | powder |
| | (log₁₀ transformed value) | | (log10 |
| | Before in-vitro | Before in-vitro | transformed |
| | digestion | digestion | value) |
| C-F-Bifido | 6.20 ± 0.28 | 3.70 ± 0.00 | 2.50 |
| Ph-F-Bifido | 8.53 ± 0.06 | 4.00 ± 0.00 | 4.53 |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from Australian Provisional Application No. 2019901142 entitled “Methods and compositions for promoting health in a subject” filed on 3 Apr. 2019, the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Agerbirk et al. (2012) Phytochemisty 77:16-45.
Alvarez-Sieiro et al. (2016) Applied Microbiology and Biotechnology 7:2939-2951.
Cai et al. (2019) Journal of Functional Foods 61, doi:103461.
Cai and Wang (2016) Food Chem 1:210:451-6.
Charoensiddhi et al. (2016) Journal of Functional foods 24:221-230.
Dosz and Jeffery (2013) Journal of Functional Foods 5:987-990.
Guzman-Lopez et al. (2009). J Ind Microbiol Biotechnol 36:11-20.
Halkier et al. (2006) Annual Reviews in Plant Biology 57:303-33.
Huang et al. (2002) Journal of agricultural and food chemistry 50(16):4437-4444.
Latte et al. (2011) Food & Chemical Toxicology, 49(12):3287-3309.
Li et al. (2012) Journal of Medicinal Plants Research 6:4796-4803.
Pacheco-Cano et al. (2017) Journal of Applied Microbiology 124(1)126-135
Singleton and Rossi (1965) American Journal of Enology and Viticulture 16:144-158.
Shen et al. (2014) Journal of the American Oil Chemists' Society, 91(8):1347-1354.
Verkerk et al. (2009) Molecular Nutrition and Food Research 53:S219-S265.
Xia and Wishart (2016) Current Protocols in Bioinformatics 55:14.10.1-14.10.91.
Zhou et al. (2006) Functional Plant Biology 33(6):585-592.

1. A method of promoting health in a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

2. The method of claim 1, wherein the Brassicaceae product increases the gastrointestinal level of one or more short chain fatty acids (SCFA) in the subject.

3. The method of claim 1 or claim 2, wherein the Brassicaceae product increases the production of one or more SCFA in the gastrointestinal tract of the subject.

4. The method of any one of claims 2 to 3, wherein the Brassicaceae product increases the production of one or more SCFA in the colon of the subject.

5. The method of any one of claims 2 to 4, wherein the SCFA is selected from one or more or all of: butyrate (butanoate), propionate (propanoate), acetate (ethanoate), formate (methanoate), isobutyrate (2-Methylpropanoate), valerate (pentanoate), isovalerate (3-methylbutanoate), caproate (hexanoate), formic acid (methanoic acid), acetic acid (ethanoic acid), propionic acid (propanoic acid), butyric acid (butanoic acid), isobutyric acid (2-methylpropanoic acid), valeric acid (pentanoic acid), isovaleric acid (3-methylbutanoic acid), and caproic acid (hexanoic acid).

6. The method of any one of claims 2 to 5, wherein the SCFA is selected from one or more or all of: butyrate, propionate and acetate.

7. The method of any one of claims 1 to 6, wherein the Brassicaceae product comprises an isothiocyanate.

8. The method of any one of claims 1 to 7, wherein the Brassicaceae product comprises live lactic acid bacteria from Brassicaceae.

9. The method of any one of claims 1 to 8, wherein promoting health comprises promoting one or more of: gut health, immune system health, cardiovascular health, central nervous system function, cognition, metabolic health, skeletal health, liver health, blood sugar control and skin health.

10. The method of any one of claims 1 to 9, wherein promoting health comprises treating or preventing one or more symptoms of a condition selected from: diabetes, inflammation, metabolic dysfunction, allergy and cancer.

11. The method of claim 10, wherein promoting gut health comprises reducing or preventing one or more symptoms of a gut health associated condition selected from one or more of: irritable bowel syndrome, inflammatory bowel disease, Crohn's disease, colorectal cancer, gut leakiness, non-alcoholic fatty liver disease, metabolic syndrome, obesity, small intestinal bacterial overgrowth (SIBO), gastroenteritis, gut microbial dysbiosis, reduced gut microbial diversity, antibiotic treatment, post-surgery recovery, food intolerance, diarrhea, gastritis, diverticulitis, flatulence, constipation, functional gut disorders and functional gastrointestinal and motility disorders.

12. The method of any one of claims 1 to 11, wherein promoting health comprises promoting health of the gut microbiome in a subject.

13. The method of claim 12, wherein promoting health of the gut microbiome comprises one or more of: increasing the level and/or activity of one or more beneficial bacteria, decreasing or maintaining the level and/or activity of one or more non-beneficial bacteria, increasing the resistance of the gut microbiome, increasing the resilience of the gut microbiome, and increasing the diversity of the gut microbiome.

14. The method of claim 13, wherein the beneficial bacteria is lactic acid bacteria.

15. The method of claim 13, wherein the non-beneficial bacteria is a pathogenic strain of E. coli.

16. The method of any one of claims 1 to 15, wherein the Brassicaceae product does not increase the total level of gastrointestinal bacteria in a subject.

17. The method of any one of claims 1 to 16, wherein the Brassicaceae product comprises a prebiotic or a prebiotic and a probiotic.

18. The method of any one of claims 1 to 17, wherein the Brassicaceae product comprises a prebiotic and a probiotic which are synbiotic.

19. The method of any one of claims 1 to 18, wherein the subject is an animal.

20. The method of claim 19, wherein the subject is a human.

21. A method of promoting the health of the gut microbiome in a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

22. A method of treating and/or preventing microbial dysbiosis in the gastrointestinal tract of a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

23. A method of treating and/or preventing inflammation in a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

24. A method of treating and/or preventing diabetes in a subject, comprising administering to the subject a Brassicaceae product fermented with lactic acid bacteria, wherein the lactic acid bacteria were derived from an isolate obtained from Brassicaceae and/or the Brassicaceae product was pre-treated prior to fermentation.

25. Use of a Brassicaceae product fermented with lactic acid bacteria in the manufacture of a medicament for promoting health in a subject, wherein the lactic acid bacteria were derived from an isolate obtained from Brassicaceae and/or the Brassicaceae product was pre-treated prior to fermentation.

26. Use of a Brassicaceae product fermented with lactic acid bacteria in the manufacture of a medicament for promoting health of the gut microbiome in a subject, wherein the lactic acid bacteria were derived from an isolate obtained from Brassicaceae and/or the Brassicaceae product was pre-treated prior to fermentation.

27. Use of a Brassicaceae product fermented with lactic acid bacteria in the manufacture of a medicament for treating and/or preventing microbial dysbiosis in the gastrointestinal tract of a subject, wherein the lactic acid bacteria were derived from an isolate obtained from Brassicaceae and/or the Brassicaceae product was pre-treated prior to fermentation.

28. Use of a Brassicaceae product fermented with lactic acid bacteria in the manufacture of a medicament for treating and/or preventing inflammation in a subject, wherein the lactic acid bacteria were derived from an isolate obtained from Brassicaceae and/or the Brassicaceae product was pre-treated prior to fermentation.

29. Use of a Brassicaceae product fermented with lactic acid bacteria in the manufacture of a medicament for treating and/or preventing diabetes in a subject, wherein the lactic acid bacteria were derived from an isolate obtained from Brassicaceae and/or the Brassicaceae product was pre-treated prior to fermentation.

30. The use of any one of claims 25 to 29 wherein the medicament comprises one or more or all of: i) a prebiotic, ii) a prebiotic and a probiotic, and iii) a prebiotic and a probiotic which are synbiotic.

31. A pharmaceutical composition comprising a Brassicaceae product fermented with lactic acid bacteria, wherein the lactic acid bacteria were derived from an isolate obtained from Brassicaceae and/or the Brassicaceae product was pre-treated prior to fermentation for use in promoting health in a subject.

32. A pharmaceutical composition comprising a Brassicaceae product fermented with lactic acid bacteria, wherein the lactic acid bacteria were derived from an isolate obtained from Brassicaceae and/or the Brassicaceae product was pre-treated prior to fermentation for use in promoting health of the gut microbiome in a subject.

33. A pharmaceutical composition comprising a Brassicaceae product with lactic acid bacteria, wherein the lactic acid bacteria were derived from an isolate obtained from Brassicaceae and/or the Brassicaceae product was pre-treated prior to fermentation for treating and/or preventing microbial dysbiosis in the gastrointestinal tract of a subject.

34. A pharmaceutical composition comprising a Brassicaceae product fermented with lactic acid bacteria, wherein the lactic acid bacteria were derived from an isolate obtained from Brassicaceae and/or the Brassicaceae product was pre-treated prior to fermentation for treating and/or preventing inflammation in a subject.

35. A pharmaceutical composition comprising a Brassicaceae product fermented with lactic acid bacteria, wherein the lactic acid bacteria were derived from an isolate obtained from Brassicaceae and/or the Brassicaceae product was pre-treated prior to fermentation for treating and/or preventing diabetes in a subject.

36. The pharmaceutical composition of any one of claims 31 to 35, wherein the composition comprises one or more or all of: i) a prebiotic, ii) a combined prebiotic and a probiotic, and iii) a prebiotic and a probiotic which are synbiotic.

37. A prebiotic composition comprising a Brassicaceae product fermented with lactic acid bacteria, wherein the lactic acid bacteria were derived from an isolate obtained from Brassicaceae and/or the Brassicaceae product was pre-treated prior to fermentation, wherein the prebiotic increases the gastrointestinal level of one or more SCFA in a subject.

38. A combined prebiotic and probiotic composition comprising:
i) a Brassicaceae product fermented with lactic acid bacteria, wherein the lactic acid bacteria were derived from an isolate obtained from Brassicaceae and/or the Brassicaceae product was pre-treated prior to fermentation; and
ii) live lactic acid bacteria.

39. The composition of claim 37 or claim 38, wherein the composition further comprises an isothiocyanate.

40. The composition of any one of claim 31 to 36, 38 or 39, wherein the composition increases the gastrointestinal level of one or more short chain fatty acids (SCFA) in a subject.

41. The composition of any one of claims 31 to 40, wherein the Brassicaceae product increases the production of one or more SCFA in the gastrointestinal tract of the subject.

42. The composition of any one of claims 31 to 41, wherein the Brassicaceae product increases the production of one or more SCFA in the colon of the subject.

43. The composition of claim 37 or claims 40 to 42, wherein the SCFA is selected from one or more or all of: butyrate (butanoate), propionate (propanoate), acetate (ethanoate), formate (methanoate), isobutyrate (2-Methylpropanoate), valerate (pentanoate), isovalerate (3-methylbutanoate), caproate (hexanoate), formic acid (methanoic acid), acetic acid (ethanoic acid), propionic acid (propanoic acid), butyric acid (butanoic acid), isobutyric acid (2-methylpropanoic acid), valeric acid (pentanoic acid), isovaleric acid (3-methylbutanoic acid), and caproic acid (hexanoic acid).

44. The composition of claim 37, or claims 40 to 43, wherein the SCFA is selected from one or more or all of: butyrate, propionate and acetate.

45. The composition of any one of claims 36 to 44, wherein the Brassicaceae product protects the probiotic during passage through the upper gasterintestinal tract.

46. The composition of any one of claims 31 to 45, wherein the Brassicaceae product increases the gastrointestinal level of live lactic acid bacteria in a subject.

47. The composition of any one of claims 31 to 46, wherein the Brassicaceae product does not increase the gastrointestinal level of E. coli in a subject.

48. The composition of any one of claims 31 to 47, wherein the Brassicaceae product does not increase the total level of gastrointestinal bacteria in a subject.

49. The method or composition of any one of claims 1 to 48, wherein the lactic acid bacteria was isolated from a broccoli.

50. The method or composition of any one of claims 1 to 49, wherein the lactic acid bacteria is selected from one or more of the genera selected from: Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and Weissella.

51. The method or composition of any one of claims 1 to 50, wherein the lactic acid bacteria is selected from one or more of: Leuconostoc mesenteroides, Lactobacillus plantarum, Lactobacillus pentosus, Lactobacillus brevis, Lactococus lactis, Pediococcus pentosaceus, Lactobacillus rhamnosus and Pedicoccus acidilacti.

52. The method or composition of any one of claims 1 to 51, wherein the lactic acid bacteria is selected from:
i) Leuconostoc mesenteroides;
ii) Lactobacillus plantarum;
iii) Lactobacillus pentosus;
iv) Lactobacillus rhamnosus;
v) a combination of i) and ii);
vi) a combination of i), ii) and iii); and
vii) a combination of i), ii) and iv).

53. The method or composition of any one of claims 1 to 52, wherein the lactic acid bacteria is selected from one or more of:
i) BF1 deposited under V17/021729 on 25 Sep. 2017 at the National Measurement Institute Australia;
ii) BF2 deposited under V17/021730 on 25 Sep. 2017 at the National Measurement Institute Australia;
iii) B1 deposited under V17/021731 on 25 Sep. 2017 at the National Measurement Institute Australia;
iv) B2 deposited under V17/021732 on 25 Sep. 2017 at the National Measurement Institute Australia;
v) B3 deposited under V17/021733 on 25 Sep. 2017 at the National Measurement Institute Australia;
vi) B4 deposited under V17/021734 on 25 Sep. 2017 at the National Measurement Institute Australia; and
vii) B5 deposited under V17/021735 on 25 Sep. 2017 at the National Measurement Institute Australia.

54. The method or composition of any one of claims 1 to 53, wherein the pre-treating comprises one or more of the following:
i) heating;
ii) macerating;
iii) microwaving;
iv) exposure to high frequency sound waves (ultrasound),
v) pulse electric field processing; and
vi) high pressure processing.

55. The method or composition of claim 54, wherein heating comprises heating the Brassicaceae product to about 50° C. to about 70° C.

56. The method or composition of any one of claims 1 to 55, wherein the Brassicaceae is selected from Brassica oleracea, Brassica balearica, Brassica carinata, Brassica elongate, Brassica fruticulosa, Brassica hilarionis, Brassica juncea, Brassica napus, Brassica narinosa, Brassica nigra, Brassica perviridis, Brassica rapa, Brassica rupestris, Brassica septiceps and Brassica tournefortii.

57. The method or composition of claim 56, wherein the Brassicaceae is Brassica oleracea.

58. The method or composition of any one of claims 1 to 57, wherein the Brassicaceae product is administered enterally.

59. The method or composition of claim 58, wherein administration is oral or rectal.

60. The method or composition of any one of claims 1 to 59, wherein the Brassicaceae product is administered topically.

61. Faecal microbiota suitable for transplantation into a subject, wherein the faecal microbiota has been isolated from a subject administered a Brassicaceae product fermented with lactic acid bacteria, wherein the lactic acid bacteria were derived from an isolate obtained from Brassicaceae and/or the Brassicaceae product was pre-treated prior to fermentation.

62. A vehicle for delivering a bioactive to a subject, wherein the vehicle comprises a Brassicaceae product fermented with lactic acid bacteria, wherein the lactic acid bacteria were derived from an isolate obtained from Brassicaceae and/or the Brassicaceae product was pre-treated prior to fermentation.

63. The vehicle of claim 62, wherein the Brassicaceae product comprises one or more or all of: i) a prebiotic, ii) a prebiotic and a probiotic, and iii) a prebiotic and a probiotic which are synbiotic.

64. The vehicle of claim 62 or claim 63, wherein the bioactive is selected from one or more or all of:
i) a fatty acid,
ii) oil,
iii) a further prebiotic, and
iv) a further probiotic.

65. The vehicle of claim 64, wherein the fatty acid is selected from an omega-3 or and omega-6 fatty acid.

66. The vehicle of claim 65, wherein the omega-3 fatty acid is selected from one or more or all of: of α-linolenic acid (ALA), eicosapentaenoic acid (EPA), docosapentaenoic acid (DPA) and docosahexaenoic acid (DHA).

67. The vehicle of claim 65, wherein the oil is selected from one or more of: fish oil, krill oil, marine oil, algal oil, microbial oil, canola oil, crustacean oil, mollusc oil, sunflower oil, avocado oil, soya oil, borage oil, evening primrose oil, safflower oil, flaxseed oil, olive oil, pumpkinseed oil, hemp seed oil, wheat germ oil, palm oil, palm oil, palm kernel oil, coconut oil, medium chain triglycerides and grapeseed oil.

68. The vehicle of claim 67, wherein the fish oil or marine oil is selected from: tuna oil, herring oil, mackerel oil, sardine oil, cod liver oil, menhaden oil, shark oil, squid oil, and squid liver oil.

69. The vehicle of claim 64, wherein the further prebiotic is selected from one or more or all of: fructo-oligosaccharides galacto-oligosaccharide, trans-galacto-oligosaccharides, oligofructose, pecticoligosaccharide, resistant starch, pectin, glucosinolate and inulin.

70. The vehicle of claim 64, wherein the further probiotic comprises one or more probiotics selected from: lactic acid bacteria, Bifidobacteria, Bacteroidetes, Baciullus, Streptococcus, Escherichia, Enterococcus and Saccharomyces.

71. The method, composition or vehicle of any one of claims 1 to 64, wherein the Brassicaceae product is in a form selected from a: liquid, emulsion, powder, capsule, tablet.

72. A method of preparing a Brassicaceae product comprising:
i) fermenting Brassicaceae material with lactic acid bacteria;
ii) adding a fatty acid and/or oil before or during step i).

73. The method of claim 72, wherein the method further comprises forming an emulsion or suspension.

74. The method of claim 72 or claim 73, wherein the Brassicaceae material is pre-treated.

75. The method of claim 74, wherein the Brassicaceae material is pre-treated with heating to about 50° C. to about 70° C.

76. The method of any one of claims 72 to 75, wherein the lactic acid bacteria were derived from an isolate obtained from Brassicaceae.

77. An emulsion or suspension produced by any one of claims 72 to 76.

78. A Brassicaceae product comprising the emulsion or suspension of claim 77.


SEQUENCE LISTING (54 sequences)

SEQ ID NO: 1 - DNA, 22 bp, Lactobacillus plantarum
tattaatggctcgcgtcattaa

SEQ ID NO: 2 - DNA, 23 bp, Lactobacillus plantarum
cgctcaaccagattagtacccag

SEQ ID NO: 3 - DNA, 10 bp, Lactobacillus plantarum
gttttttttg

SEQ ID NO: 4 - DNA, 11 bp, Lactobacillus plantarum
gtttttttttg

SEQ ID NO: 5 - DNA, 23 bp, Lactobacillus plantarum
caccaccaggccgattgtggcga

SEQ ID NO: 6 - DNA, 34 bp, Lactobacillus plantarum
cttgccgaaattcgacaaacaaccctcggattgt

SEQ ID NO: 7 - DNA, 70 bp, Lactobacillus plantarum
tataaaaaaagcgacccccgttcattaacggtgccgctcacagatcattattagtgaaaatcacccggca

SEQ ID NO: 8 - DNA, 14 bp, Lactobacillus mesenteroides
ggtatgggatggga

SEQ ID NO: 9 - DNA, 11 bp, Leuconostoc mesenteroides
gttttttttta

SEQ ID NO: 10 - DNA, 10 bp, Leuconostoc mesenteroides
gttttttttc

SEQ ID NO: 11 - DNA, 21 bp, Leuconostoc mesenteroides
tagctgcaagtgctgcaagtg

SEQ ID NO: 12 - DNA, 12 bp, Leuconostoc mesenteroides
cagctgcaagtg

SEQ ID NO: 13 - DNA, 10 bp, Leuconostoc mesenteroides
cagattaacg

SEQ ID NO: 14 - DNA, 10 bp, Leuconostoc mesenteroides
aaaatcaaaa

SEQ ID NO: 15 - DNA, 14 bp, Leuconostoc mesenteroides
cgaaacgctcattc

SEQ ID NO: 16 - DNA, 14 bp, Leuconostoc mesenteroides
tgagacactaatta

SEQ ID NO: 17 - DNA, 17 bp, Leuconostoc mesenteroides
atttaagttcagtcaca

SEQ ID NO: 18 - DNA, 17 bp, Leuconostoc mesenteroides
ctacaatatcacttccc

SEQ ID NO: 19 - DNA, 12 bp, Leuconostoc mesenteroides
taaagcctcttg

SEQ ID NO: 20 - DNA, 12 bp, Leuconostoc mesenteroides
cagagcagcttc

SEQ ID NO: 21 - DNA, 20 bp, Leuconostoc mesenteroides
tggctcctctatttgtcttt

SEQ ID NO: 22 - DNA, 20 bp, Leuconostoc mesenteroides
aggcacctttagtcgtttta

SEQ ID NO: 23 - DNA, 10 bp, Leuconostoc mesenteroides
agctgtgacc

SEQ ID NO: 24 - DNA, 10 bp, Leuconostoc mesenteroides
ggcagtcact

SEQ ID NO: 25 - DNA, 10 bp, Leuconostoc mesenteroides
aaccaatcct

SEQ ID NO: 26 - DNA, 10 bp, Leuconostoc mesenteroides
tacaaaacca

SEQ ID NO: 27 - DNA, 15 bp, Leuconostoc mesenteroides
caaaaaaaaaaaaac

SEQ ID NO: 28 - DNA, 14 bp, Leuconostoc mesenteroides
caaaaaaaacaaac

SEQ ID NO: 29 - DNA, 13 bp, Leuconostoc mesenteroides
tgtgttcaaagat

SEQ ID NO: 30 - DNA, 13 bp, Leuconostoc mesenteroides
catatttagagac

SEQ ID NO: 31 - DNA, 13 bp, Leuconostoc mesenteroides
ttttaaaccgtca

SEQ ID NO: 32 - DNA, 13 bp, Leuconostoc mesenteroides
cttcaaatcatcg

SEQ ID NO: 33 - DNA, 10 bp, Leuconostoc mesenteroides
gaaagacgct

SEQ ID NO: 34 - DNA, 10 bp, Leuconostoc mesenteroides
aaaggatgcc

SEQ ID NO: 35 - DNA, 13 bp, Leuconostoc mesenteroides
ggccgctagcatg

SEQ ID NO: 36 - DNA, 13 bp, Leuconostoc mesenteroides
tgcagccaacata

SEQ ID NO: 37 - DNA, 17 bp, Leuconostoc mesenteroides
acccgatgggttgtatt

SEQ ID NO: 38 - DNA, 15 bp, Leuconostoc mesenteroides
gccactaggctgcat

SEQ ID NO: 39 - DNA, 28 bp, Leuconostoc mesenteroides
ggctgcaaacgctgcacgcaggcgcagc

SEQ ID NO: 40 - DNA, 28 bp, Leuconostoc mesenteroides
agcagcgaaagcagcgcgtaaacgaagt

SEQ ID NO: 41 - DNA, 22 bp, Leuconostoc mesenteroides
ctggggtatcaaaacggttaca

SEQ ID NO: 42 - DNA, 22 bp, Leuconostoc mesenteroides
ttgtggaattaatactgtcact

SEQ ID NO: 43 - DNA, 15 bp, Leuconostoc mesenteroides
attacctttgataac

SEQ ID NO: 44 - DNA, 15 bp, Leuconostoc mesenteroides
gttatcaaaggtaat

SEQ ID NO: 45 - DNA, 10 bp, Leuconostoc mesenteroides
caatgaaccg

SEQ ID NO: 46 - DNA, 10 bp, Leuconostoc mesenteroides
tagagagcca

SEQ ID NO: 47 - DNA, 10 bp, Leuconostoc mesenteroides
tgtcacttga

SEQ ID NO: 48 - DNA, 10 bp, Leuconostoc mesenteroides
agttacctgg

SEQ ID NO: 49 - DNA, 12 bp, Leuconostoc mesenteroides
tgtgttaaatac

SEQ ID NO: 50 - DNA, 12 bp, Leuconostoc mesenteroides
agtgctatattt

SEQ ID NO: 51 - DNA, 14 bp, Leuconostoc mesenteroides
gaccccgttcaggc

SEQ ID NO: 52 - DNA, 14 bp, Leuconostoc mesenteroides
atcctcgctccggt

SEQ ID NO: 53 - DNA, 12 bp, Leuconostoc mesenteroides
gtttcagaaaaa

SEQ ID NO: 54 - DNA, 12 bp, Leuconostoc mesenteroides
atgtcggaagag